SEQUENCE LISTING (114 sequences)

SEQ ID NO: 1 - DNA, 22 bp, Homo sapiens
aagagagattggatttggaacc

SEQ ID NO: 2 - DNA, 22 bp, Homo sapiens
ccagaagcccaagaagattgta

SEQ ID NO: 3 - DNA, 19 bp, Homo sapiens
aatcctggtgtcaaggaag

SEQ ID NO: 4 - DNA, 19 bp, Homo sapiens
ggaccgattttaccgatcc

SEQ ID NO: 5 - DNA, 21 bp, Homo sapiens
acagtccagatagtcgtatgt

SEQ ID NO: 6 - DNA, 17 bp, Homo sapiens
gtctccgccatccctat

SEQ ID NO: 7 - DNA, 19 bp, Homo sapiens
actggtgtaacaggaacat

SEQ ID NO: 8 - DNA, 17 bp, Homo sapiens
tttggaaggactgcgct

SEQ ID NO: 9 - DNA, 17 bp, Homo sapiens
cacgtcatctcccgttc

SEQ ID NO: 10 - DNA, 18 bp, Homo sapiens
attgaacttcccacacga

SEQ ID NO: 11 - DNA, 18 bp, Homo sapiens
ggaacagactgtcaccat

SEQ ID NO: 12 - DNA, 19 bp, Homo sapiens
tcagagtgtgtggtcaggc

SEQ ID NO: 13 - DNA, 17 bp, Homo sapiens
gggacagcttcaacact

SEQ ID NO: 14 - DNA, 18 bp, Homo sapiens
cctgtgaacagccctatg

SEQ ID NO: 15 - DNA, 17 bp, Homo sapiens
ttctgggcacggtgaag

SEQ ID NO: 16 - DNA, 21 bp, Homo sapiens
ggccaaactagagcacgaata

SEQ ID NO: 17 - DNA, 19 bp, Homo sapiens
tcagcaagaaggagatgcc

SEQ ID NO: 18 - DNA, 21 bp, Homo sapiens
gtgctccctctccattaagta

SEQ ID NO: 19 - DNA, 20 bp, Homo sapiens
caagttcaggagaactcgac

SEQ ID NO: 20 - DNA, 19 bp, Homo sapiens
ggctgtggttatgcgatag

SEQ ID NO: 21 - DNA, 18 bp, Homo sapiens
acccgaggaacaacctta

SEQ ID NO: 22 - DNA, 18 bp, Homo sapiens
ccctctccattccctaca

SEQ ID NO: 23 - DNA, 17 bp, Homo sapiens
cagagcgccaggcatta

SEQ ID NO: 24 - DNA, 18 bp, Homo sapiens
ccactggctgaggtgtta

SEQ ID NO: 25 - DNA, 17 bp, Homo sapiens
tgggcgagtctacgatg

SEQ ID NO: 26 - DNA, 18 bp, Homo sapiens
ctttctgccctggagatg

SEQ ID NO: 27 - DNA, 19 bp, Homo sapiens
gcgccatttgctagagata

SEQ ID NO: 28 - DNA, 19 bp, Homo sapiens
agagaagatgggcagaaag

SEQ ID NO: 29 - DNA, 17 bp, Homo sapiens
gcccagatcatccgtca

SEQ ID NO: 30 - DNA, 17 bp, Homo sapiens
accacaaggacttcgac

SEQ ID NO: 31 - DNA, 17 bp, Homo sapiens
gctccgctgctatcttt

SEQ ID NO: 32 - DNA, 17 bp, Homo sapiens
agcggccaggtggatta

SEQ ID NO: 33 - DNA, 18 bp, Homo sapiens
atgggctttgggagcata

SEQ ID NO: 34 - DNA, 18 bp, Homo sapiens
gacctggatgccaagcta

SEQ ID NO: 35 - DNA, 17 bp, Homo sapiens
ccggctcttggaagttg

SEQ ID NO: 36 - DNA, 20 bp, Homo sapiens
acgcggatcgagtttgataa

SEQ ID NO: 37 - DNA, 17 bp, Homo sapiens
cgcaagtcccagaagat

SEQ ID NO: 38 - DNA, 17 bp, Homo sapiens
cgcggatacgatgtcac

SEQ ID NO: 39 - DNA, 17 bp, Homo sapiens
gaactcggcctatcgct

SEQ ID NO: 40 - DNA, 20 bp, Homo sapiens
tctgacctcatcatcggcaa

SEQ ID NO: 41 - DNA, 20 bp, Homo sapiens
gaggtgaagcaaactacgga

SEQ ID NO: 42 - DNA, 17 bp, Homo sapiens
actctccacaaagctcg

SEQ ID NO: 43 - DNA, 22 bp, Homo sapiens
ggatttcagctaccagttactt

SEQ ID NO: 44 - DNA, 17 bp, Homo sapiens
ttcgtcctggtggatcg

SEQ ID NO: 45 - DNA, 22 bp, Homo sapiens
agtgattgatgtgtttgctatg

SEQ ID NO: 46 - DNA, 20 bp, Homo sapiens
caaagccaagccactcactc

SEQ ID NO: 47 - DNA, 17 bp, Homo sapiens
ctcggcagtcctgtttc

SEQ ID NO: 48 - DNA, 18 bp, Homo sapiens
acacctggtacgtcagaa

SEQ ID NO: 49 - DNA, 20 bp, Homo sapiens
atgcccaagagaatcgtaaa

SEQ ID NO: 50 - DNA, 19 bp, Homo sapiens
atgagtccaaagcacacga

SEQ ID NO: 51 - DNA, 22 bp, Homo sapiens
tgagattgaggatgaagctgag

SEQ ID NO: 52 - DNA, 17 bp, Homo sapiens
ccgactcaacgtgagac

SEQ ID NO: 53 - DNA, 17 bp, Homo sapiens
gtgccctctccttttcg

SEQ ID NO: 54 - DNA, 18 bp, Homo sapiens
cgttctttttcgcaacgg

SEQ ID NO: 55 - DNA, 17 bp, Homo sapiens
ggtgtgccactgaagat

SEQ ID NO: 56 - DNA, 17 bp, Homo sapiens
gtgtcgtggtggtcatt

SEQ ID NO: 57 - DNA, 17 bp, Homo sapiens
gcatgaagacagtggct

SEQ ID NO: 58 - DNA, 17 bp, Homo sapiens
ttcttgcgactcacgct

SEQ ID NO: 59 - DNA, 24 bp, Homo sapiens
gctcctcaaacatctttgtgttca

SEQ ID NO: 60 - DNA, 20 bp, Homo sapiens
gaccactgtgggtcattatt

SEQ ID NO: 61 - DNA, 17 bp, Homo sapiens
gaaatctctggccgctc

SEQ ID NO: 62 - DNA, 21 bp, Homo sapiens
actgggcatcataagaaatcc

SEQ ID NO: 63 - DNA, 19 bp, Homo sapiens
actgaacagaagacttcgt

SEQ ID NO: 64 - DNA, 20 bp, Homo sapiens
aacctccaagtggaaattct

SEQ ID NO: 65 - DNA, 22 bp, Homo sapiens
tcggtctttcaaatcgggatta

SEQ ID NO: 66 - DNA, 18 bp, Homo sapiens
ctgctgtcacaggacaat

SEQ ID NO: 67 - DNA, 19 bp, Homo sapiens
aaggtaaagccagactcca

SEQ ID NO: 68 - DNA, 17 bp, Homo sapiens
gggagcgtagggttaag

SEQ ID NO: 69 - DNA, 22 bp, Homo sapiens
cagtgtattctgcacaatcaac

SEQ ID NO: 70 - DNA, 21 bp, Homo sapiens
gttccaggatgttggactttc

SEQ ID NO: 71 - DNA, 18 bp, Homo sapiens
ggaaagtgtgtcggagat

SEQ ID NO: 72 - DNA, 18 bp, Homo sapiens
aggcaacatcattccctc

SEQ ID NO: 73 - DNA, 22 bp, Homo sapiens
gtcaacacccatcttcttgaaa

SEQ ID NO: 74 - DNA, 18 bp, Homo sapiens
cgtagtggaagacggaaa

SEQ ID NO: 75 - DNA, 23 bp, Homo sapiens
ctggtgtagaattaggagacgta

SEQ ID NO: 76 - DNA, 17 bp, Homo sapiens
ggcatcaagagagaggc

SEQ ID NO: 77 - DNA, 24 bp, Homo sapiens
gataaagagttacaagctcctctg

SEQ ID NO: 78 - DNA, 17 bp, Homo sapiens
tctaggccttgacggat

SEQ ID NO: 79 - DNA, 19 bp, Homo sapiens
tttgggcaaacctcggtaa

SEQ ID NO: 80 - DNA, 17 bp, Homo sapiens
gcacagcaaatgccact

SEQ ID NO: 81 - DNA, 23 bp, Homo sapiens
cttgtctttccctactgtcttac

SEQ ID NO: 82 - DNA, 18 bp, Homo sapiens
cttgttccagcagaacct

SEQ ID NO: 83 - DNA, 18 bp, Homo sapiens
cagtcctctgcaccgtta

SEQ ID NO: 84 - DNA, 18 bp, Homo sapiens
catccagatccctcacat

SEQ ID NO: 85 - DNA, 19 bp, Homo sapiens
ccaagacacagccagtaat

SEQ ID NO: 86 - DNA, 18 bp, Homo sapiens
tttccagccctcgtagtc

SEQ ID NO: 87 - DNA, 17 bp, Homo sapiens
gggacacagggaagaac

SEQ ID NO: 88 - DNA, 17 bp, Homo sapiens
gtctgccactctgcaac

SEQ ID NO: 89 - DNA, 17 bp, Homo sapiens
gtcggctgacgctttga

SEQ ID NO: 90 - DNA, 23 bp, Homo sapiens
gaacaagtcagtctagggaatac

SEQ ID NO: 91 - DNA, 21 bp, Homo sapiens
tgctttcgataagtccagaca

SEQ ID NO: 92 - DNA, 18 bp, Homo sapiens
cctctgaggctggaaaca

SEQ ID NO: 93 - DNA, 19 bp, Homo sapiens
atccactgatcttccttgc

SEQ ID NO: 94 - DNA, 19 bp, Homo sapiens
cagtgctgcttcagacaca

SEQ ID NO: 95 - DNA, 21 bp, Homo sapiens
cctttcttcaagggtaaaggc

SEQ ID NO: 96 - DNA, 20 bp, Homo sapiens
tcgaatttctctcctcccat

SEQ ID NO: 97 - DNA, 18 bp, Homo sapiens
ctgagtccacacaggttt

SEQ ID NO: 98 - DNA, 23 bp, Homo sapiens
cccatacttgttgatggcaatta

SEQ ID NO: 99 - DNA, 18 bp, Homo sapiens
tcctgcgtgtgttctact

SEQ ID NO: 100 - DNA, 19 bp, Homo sapiens
agtcatcatgtacccagca

SEQ ID NO: 101 - DNA, 20 bp, Homo sapiens
cccaggatactctcttcctt

SEQ ID NO: 102 - DNA, 18 bp, Homo sapiens
cactggatcaactgcctc

SEQ ID NO: 103 - DNA, 19 bp, Homo sapiens
cagctgtcacacccagagc

SEQ ID NO: 104 - DNA, 17 bp, Homo sapiens
cgtatggtgcagggtca

SEQ ID NO: 105 - DNA, 20 bp, Homo sapiens
tctggactgtctggttgaat

SEQ ID NO: 106 - DNA, 19 bp, Homo sapiens
cctgtacaccaagcttcat

SEQ ID NO: 107 - DNA, 19 bp, Homo sapiens
ccatgcccactttcttgta

SEQ ID NO: 108 - DNA, 20 bp, Homo sapiens
cattggtggtgaagctcttg

SEQ ID NO: 109 - DNA, 18 bp, Homo sapiens
cgtggactgagatgcatt

SEQ ID NO: 110 - DNA, 21 bp, Homo sapiens
ttcatgtcgttgaacaccttg

SEQ ID NO: 111 - DNA, 21 bp, Homo sapiens
cattttggcttttaggggtag

SEQ ID NO: 112 - DNA, 17 bp, Homo sapiens
ggcagaagcgagacttt

SEQ ID NO: 113 - DNA, 17 bp, Homo sapiens
gcacataggaggtggca

SEQ ID NO: 114 - DNA, 17 bp, Homo sapiens
gcggactttaccgtgac

(19) United States
(12) Patent Application Publication
(10) Pub. No.: US 2022/0243283 A1
(43) Pub. Date: Aug. 4, 2022

(54) METHODS FOR TYPING OF LUNG CANCER

(71) Applicants: GeneCentric Therapeutics, Inc., Durham, NC (US); The University of North Carolina at Chapel Hill, Chapel Hill, NC (US)

(72) Inventors: Hawazin FARUKI, Durham, NC (US); Myla LAI-GOLDMAN, Durham, NC (US); Greg MAYHEW, Durham, NC (US); Charles PEROU, Carrboro, NC (US); David Neil HAYES, Chapel Hill, NC (US)

(21) Appl. No.: 17/725,936

(22) Filed: Apr. 21, 2022

Related U.S. Application Data
(63) Continuation of application No. 17/471,716, filed on Sep. 10, 2021, now abandoned.
Application No. 17/471,716 is a continuation of application No. 17/144,644, filed on Jan. 8, 2021, now abandoned.
Application No. 17/144,644 is a continuation of application No. 16/887,241, filed on May 29, 2020, now abandoned.
Application No. 16/887,241 is a continuation of application No. 15/566,363, filed on Oct. 13, 2017, now abandoned.
(60) Provisional application No. 62/147,547, filed on Apr. 14, 2015.

(51) Int. Cl. C12Q 1/6886

(52) U.S. Cl. CPC C12Q 1/6886; C12Q 2600/158; C12Q 2600/118; C12Q 2600/112

(57) ABSTRACT

Methods and compositions are provided for the molecular subtyping of lung cancer samples. Specifically, a method of assessing whether a patient's adenocarcinoma lung cancer subtype is terminal respiratory unit (TRU), proximal inflammatory (PI), or proximal proliferative (PP) is provided herein. The method entails detecting the levels of the classifier biomarkers of Table 1-Table 6 or a subset thereof at the nucleic acid level, in a lung cancer sample obtained from the patient. Based in part on the levels of the classifier biomarkers, the lung cancer sample is classified as a TRU, PI, or PP sample.

### CROSS REFERENCE TO U.S. NON-PROVISIONAL APPLICATONS

This application is a continuation of U.S. application Ser. No. 17/471,716, filed Sep. 10, 2021, which is a continuation of U.S. application Ser. No. 17/144,644, filed Jan. 8, 2021, which is a continuation of U.S. application Ser. No. 16/887,241, filed May 29, 2020, which is a continuation of U.S. application Ser. No. 15/566,363, filed Oct. 13, 2017, which is a national phase of International Application No. PCT/US16/27503, filed Apr. 14, 2016, which claims priority from U.S. Provisional Application Ser. No. 62/147,547, filed Apr. 14, 2015, each of which is incorporated by reference herein in its entirety for all purposes.

### STATEMENT REGARDING SEQUENCE LISTING

The contents of the text file submitted electronically herewith are incorporated herein by reference in their entirety: A computer readable format copy of the Sequence Listing (filename: GNCN_007_05US_SeqList_ST25.txt, date recorded: Apr. 21, 2022, file size ˜17,538 bytes).

### BACKGROUND OF THE INVENTION

Lung cancer is the leading cause of cancer death in the United States and over 220,000 new lung cancer cases are identified each year. Lung cancer is a heterogeneous disease with subtypes generally determined by histology (small cell, non-small cell, carcinoid, adenocarcinoma, and squamous cell carcinoma). Differentiation among various morphologic subtypes of lung cancer is essential in guiding patient management and additional molecular testing is used to identify specific therapeutic target markers. Variability in morphology, limited tissue samples, and the need for assessment of a growing list of therapeutically targeted markers pose challenges to the current diagnostic standard. Studies of histologic diagnosis reproducibility have shown limited intra-pathologist agreement and inter-pathologist agreement.

While new therapies are increasingly directed toward specific subtypes of lung cancer (bevacizumab and pemetrexed), studies of histologic diagnosis reproducibility have shown limited intra-pathologist agreement and even less inter-pathologist agreement. Poorly differentiated tumors, conflicting immunohistochemistry results, and small volume biopsies in which only a limited number of stains can be performed continue to pose challenges to the current diagnostic standard (Travis and Rekhtman Sem Resp and Crit Care Med 2011; 32(1): 22-31; Travis et al. Arch Pathol Lab Med 2013; 137(5):668-84; Tang et al. J Thorac Dis 2014; 6(S5):S489-S501).

A recent example involving expert pathology re-review of lung cancer samples submitted to the TCGA Lung Cancer genome project led to the reclassification of 15-20% of lung tumors submitted, confirming the ongoing challenge of morphology-based diagnoses. (Cancer Genome Atlas Research Network. “Comprehensive genomic characterization of squamous cell lung cancers.” Nature 489.7417 (2012): 519-525; Cancer Genome Atlas Research Network. Comprehensive molecular profiling of lung adenocarcinoma. Nature 511.7511 (2014): 543-550, each of which is incorporated by reference herein in its entirety). Thus a need exists for a more reliable means for determining lung cancer subtype. The present invention addresses this and other needs.

### SUMMARY OF THE INVENTION

In one aspect, a method of assessing whether a patient's adenocarcinoma lung cancer subtype is squamoid (proximal inflammatory), bronchoid (terminal respiratory unit) or magnoid (proximal proliferative). In one embodiment, the method comprises probing the levels of at least five classifier biomarkers of the classifier biomarkers of Table 1A, Table 1B, Table 1C, Table 2, Table 3, Table 4, Table 5 or Table 6 at the nucleic acid level, in a lung cancer sample obtained from the patient. The probing step, in one embodiment, comprises mixing the sample with five or more oligonucleotides that are substantially complementary to portions of cDNA molecules of the at least five classifier biomarkers of Table 1A, Table 1B, Table 1C, Table 2, Table 3, Table 4, Table 5 or Table 6 under conditions suitable for hybridization of the five or more oligonucleotides to their complements or substantial complements; detecting whether hybridization occurs between the five or more oligonucleotides to their complements or substantial complements; and obtaining hybridization values of the at least five classifier biomarkers based on the detecting step. The hybridization values of the at least five classifier biomarkers are then compared to reference hybridization value(s) from at least one sample training set, wherein the at least one sample training set comprises, (i) hybridization value(s) of the at least five biomarkers from a sample that overexpresses the at least five biomarkers, or overexpresses a subset of the at least five biomarkers, (ii) hybridization values from a reference squamoid (proximal inflammatory), bronchoid (terminal respiratory unit) or magnoid (proximal proliferative) sample, or (iii) hybridization values from an adenocarcinoma free lung sample. The adenocarcinoma lung cancer sample is classified as a squamoid (proximal inflammatory), bronchoid (terminal respiratory unit) or a magnoid (proximal proliferative) subtype based on the results of the comparing step. In one embodiment, the comparing step comprises determining a correlation between the hybridization values of the at least five classifier biomarkers and the reference hybridization values. In one embodiment, the comparing step further comprises determining an average expression ratio of the at least five biomarkers and comparing the average expression ratio to an average expression ratio of the at least five biomarkers, obtained from the references values in the sample training set. In one embodiment, the probing step comprises isolating the nucleic acid or portion thereof prior to the mixing step. In a further embodiment, the hybridization comprises hybridization of a cDNA to a cDNA, thereby forming a non-natural complex; or hybridization of a cDNA to an mRNA, thereby forming a non-natural complex. In even a further embodiment, the probing step comprises amplifying the nucleic acid in the sample. In one embodiment, the lung cancer sample comprises lung cells embedded in paraffin. In one embodiment, the lung cancer sample is a fresh frozen sample. In one embodiment, the lung cancer sample is selected from a formalin-fixed, paraffin-embedded (FFPE) lung tissue sample, fresh and a frozen tissue sample.

In another aspect, provided herein is a method for assessing whether a lung tissue sample from a human patient is a squamoid (proximal inflammatory), bronchoid (terminal respiratory unit) or magnoid (proximal proliferative) adenocarcinoma lung cancer subtype. In one embodiment, the method comprises detecting expression levels of at least five of the classifier biomarkers of Table 1A, Table 1B, Table 1C, Table 2, Table 3, Table 4, Table 5 or Table 6 at the nucleic acid level by RNA-seq, a reverse transcriptase polymerase chain reaction (RT-PCR) or a hybridization assay with oligonucleotides specific to the classifier biomarkers; comparing the detected levels of expression of the at least five of the classifier biomarkers of Table 1A, Table 1B, Table 1C, Table 2, Table 3, Table 4, Table 5 or Table 6 to the expression levels of the at least five of the classifier biomarkers from at least one sample training set. In one embodiment, the at least one sample training set comprises, (i) expression levels(s) of the at least five biomarkers from a sample that overexpresses the at least five biomarkers, or overexpresses a subset of the at least five biomarkers, (ii) expression levels from a reference squamoid (proximal inflammatory), bronchoid (terminal respiratory unit) or magnoid (proximal proliferative) sample, or (iii) expression levels from an adenocarcinoma free lung sample; and classifying the lung tissue sample as a squamoid (proximal inflammatory), bronchoid (terminal respiratory unit) or a magnoid (proximal proliferative) subtype based on the results of the comparing step. In one embodiment, the comparing step comprises applying a statistical algorithm which comprises determining a correlation between the expression data obtained from the lung tissue sample and the expression data from the at least one training set(s); and classifying the lung tissue sample as a squamoid (proximal inflammatory), bronchoid (terminal respiratory unit) or a magnoid (proximal proliferative) subtype based on the results of the statistical algorithm. In one embodiment, the comparing step further comprises determining an average expression ratio of the at least five biomarkers and comparing the average expression ratio to an average expression ratio of the at least five biomarkers, obtained from the references values in the sample training set. In one embodiment, the lung tissue sample is selected from a formalin-fixed, paraffin-embedded (FFPE) lung tissue sample, fresh and a frozen tissue sample.

In yet another aspect, provided herein is a method for determining a disease outcome for a patient suffering from lung cancer, the method comprising: determining a subtype of the lung cancer through gene expression analysis of a first sample obtained from the patient to produce a gene expression based subtype; determining the subtype of the lung cancer through a morphological analysis of a second sample obtained from the patient to produce a morphological based subtype; and comparing the gene expression based subtype to the morphological based subtype, wherein a presence or absence of concordance between the gene expression based subtype and the morphological based subtype is predictive of the disease outcome. In one embodiment, discordance between the gene expression based subtype and morphological based subtype is predictive of a poor disease outcome. In one embodiment, the disease outcome is overall survival. In one embodiment, the gene expression base subtype and/or morphological based subtype is adenocarcinoma, squamous cell carcinoma, or neuroendocrine. In one embodiment, the neuroendocrine encompasses small cell carcinoma and carcinoid. In one embodiment, the first sample and/or the second sample is a formalin-fixed, paraffin-embedded (FFPE) lung tissue sample, fresh, or a frozen tissue sample. In one embodiment, the first sample and the second sample are portions of an identical sample. In one embodiment, the gene expression analysis comprises determining expression levels of at least five classifier biomarkers in Table 1A, Table 1B, Table 1C, Table 2, Table 3, Table 4, Table 5 or Table 6 at a nucleic acid level in the first sample by performing RNA sequencing, reverse transcriptase polymerase chain reaction (RT-PCR) or hybridization based analyses. In one embodiment, the RT-PCR is quantitative real time reverse transcriptase polymerase chain reaction (qRT-PCR). In one embodiment, the RT-PCR is performed with primers specific to the at least five classifier biomarkers; comparing the detected levels of expression of the at least five classifier biomarkers of Table 1A, Table 1B, Table 1C, Table 2, Table 3, Table 4, Table 5 or Table 6 to the expression of the at least five classifier biomarkers in at least one sample training set(s), wherein the at least one sample training set comprises expression data of the at least five classifier biomarkers of Table 1A, Table 1B, Table 1C, Table 2, Table 3, Table 4, Table 5 or Table 6 from a reference adenocarcinoma sample, expression data of the at least five classifier biomarkers of Table 1A, Table 1B, Table 1C, Table 2, Table 3, Table 4, Table 5 or Table 6 from a reference squamous cell carcinoma sample, expression data of the at least five classifier biomarkers of Table 1A, Table 1B, Table 1C, Table 2, Table 3, Table 4, Table 5 or Table 6 from a reference neuroendocrine sample, or a combination thereof; and classifying the first sample as an adenocarcinoma, squamous cell carcinoma, or a neuroendocrine subtype based on the results of the comparing step. In one embodiment, the comparing step comprises applying a statistical algorithm which comprises determining a correlation between the expression data obtained from the first sample and the expression data from the at least one training set(s); and classifying the first sample as an adenocarcinoma, squamous cell carcinoma, or a neuroendocrine subtype based on the results of the statistical algorithm. In one embodiment, the primers specific for the at least five classifier biomarkers are forward and reverse primers listed in Table 1A, Table 1B, Table 1C, Table 2, Table 3, Table 4, Table 5 or Table 6. In one embodiment, the hybridization analysis comprises: (a) probing the levels of at least five classifier biomarkers of Table 1A, Table IB, Table 1C, Table 2, Table 3, Table 4, Table 5 or Table 6 in a lung cancer sample obtained from the patient at the nucleic acid level, wherein the probing step comprises; (i) mixing the sample with five or more oligonucleotides that are substantially complementary to portions of nucleic acid molecules of the at least five classifier biomarkers of Table 1A, Table IB, Table 1C, Table 2, Table 3, Table 4, Table 5 or Table 6 under conditions suitable for hybridization of the five or more oligonucleotides to their complements or substantial complements; (ii) detecting whether hybridization occurs between the five or more oligonucleotides to their complements or substantial complements; (iii) obtaining hybridization values of the at least five classifier biomarkers based on the detecting step; (b) comparing the hybridization values of the at least five classifier biomarkers to reference hybridization value(s) from at least one sample training set, wherein the at least one sample training set comprises hybridization values from a reference adenocarcinoma sample, hybridization values from a reference squamous cell carcinoma sample, hybridization values from a reference neuroendocrine sample, or a combination thereof; and (c) classifying the lung cancer sample as a adenocarcinoma, squamous cell carcinoma, or a neuroendocrine subtype based on the results of the comparing step. In one embodiment, the comparing step comprises determining a correlation between the hybridization values of the at least five classifier biomarkers and the reference hybridization values. In one embodiment, the comparing step further comprises determining an average expression ratio of the at least five biomarkers and comparing the average expression ratio to an average expression ratio of the at least five biomarkers, obtained from the references values in the sample training set. In one embodiment, the probing step comprises isolating the nucleic acid or portion thereof prior to the mixing step. In one embodiment, the hybridization comprises hybridization of a cDNA probe to a cDNA biomarker, thereby forming a non-natural complex. In one embodiment, the hybridization comprises hybridization of a cDNA probe to an mRNA biomarker, thereby forming a non-natural complex. In one embodiment, the morphological analysis of the second sample is a histological analysis.

In one embodiment, the at least five of the classifier biomarkers of any of the aspects provided above comprise at least 10 biomarkers, at least 20 biomarkers or at least 30 biomarkers of Table 1A, Table 1B or Table 1C. In one embodiment, the at least five of the classifier biomarkers comprise at least 10 biomarkers, at least 20 biomarkers or at least 30 biomarkers of Table 2. In one embodiment, the at least five of the classifier biomarkers comprise at least 10 biomarkers, at least 20 biomarkers or at least 30 biomarkers of Table 3. In one embodiment, the at least five of the classifier biomarkers comprise the 6 biomarkers of Table 4. In one embodiment, the at least five of the classifier biomarkers comprise the 6 biomarkers of Table 5. In one embodiment, the at least five of the classifier biomarkers comprise at least 10 biomarkers, at least 20 biomarkers or at least 30 biomarkers of Table 6. In one embodiment, the at least five of the classifier biomarkers comprise from about 10 to about 30 classifier biomarkers, or from about 15 to about 40 classifier biomarkers of Table 1A, Table 1B or Table 1C. In one embodiment, the at least five of the classifier biomarkers comprise from about 10 to about 30 classifier biomarkers, or from about 15 to about 40 classifier biomarkers of Table 2. In one embodiment, the at least five of the classifier biomarkers comprise from about 10 to about 30 classifier biomarkers, or from about 15 to about 40 classifier biomarkers of Table 3. In one embodiment, the at least five classifier biomarkers comprise from about 5 to about 30 classifier biomarkers, or from about 10 to about 30 classifier biomarkers of Table 6. In one embodiment, the at least five of the classifier biomarkers comprise each of the classifier biomarkers set forth in Table 1A, Table 1B or Table 1C. In one embodiment, the at least five of the classifier biomarkers comprise each of the classifier biomarkers set forth in Table 2. In one embodiment, the at least five of the classifier biomarkers comprise each of the classifier biomarkers set forth in Table 3. In one embodiment, the at least five of the classifier biomarkers comprise each of the classifier biomarkers set forth in Table 6. In one embodiment, the at least five of the classifier biomarkers comprise each of the classifier biomarkers set forth in Table 1A. In one embodiment, the at least five of the classifier biomarkers comprise each of the classifier biomarkers set forth in Table 1B. In one embodiment, the at least five of the classifier biomarkers comprise each of the classifier biomarkers set forth in Table 1C.

### BRIEF DESCRIPTION OF THE DRAWINGS

FIGS. 1A-1D illustrate exemplary gene expression heatmaps for adenocarcinoma (FIG. 1A), squamous cell carcinoma (FIG. 1B), small cell carcinoma (FIG. 1C), and carcinoid (FIG. 1D).

FIG. 2 illustrates a heatmap of gene expression hierarchical clustering for FFPE RT-PCR gene expression dataset.

FIG. 3 illustrates a comparison of path review and LSP prediction for 77 FFPE samples. Each rectangle represents a single sample ordered by sample number. Arrows indicate 6 samples that disagreed with the original diagnosis by both pathology review and gene expression (for sample details see Table 18).

FIGS. 4-7 illustrates Kaplan Meier plots showing the predicted lung cancer subtype AD, SQ, or NE as a function of overall survival for 5 years for 3 independent AD datasets: Director's Challenge (Shedden et al; FIG. 4), TCGA RNAseq data (FIG. 5), Tomida et al. array data (FIG. 6) or pooled (FIG. 7) assigned a LSP gene expression subtype across all stages.

FIGS. 8-11 illustrates Kaplan Meier plots showing the predicted lung cancer subtype AD, SQ, or NE as a function of overall survival for 5 years for 3 independent AD datasets: Director's Challenge (Shedden et al; FIG. 8), TCGA RNAseq data (FIG. 9), Tomida et al. array data (FIG. 10) or pooled (FIG. 11) assigned a LSP gene expression subtype across stages I and II.

FIG. 12 illustrates the proliferation score (11 gene PAM50 signature) is higher in AD-NE/SQ compared to AD-AD in all 3 datasets shown in FIGS. 4-6.

FIG. 13 illustrates gene mutation prevalence in histology-gene expression concordant (AD-AD) as compared to discordant (AD-NE/SQ) samples using Fisher's exact test.

FIG. 14 illustrates reduction in lung adenocarcinoma prognostic strength following exclusion of histologically defined adenocarcinoma samples that are NE or SQ by LSP gene expression (AD-NE/SQ).

FIG. 15 illustrates the Cox proportional hazard models of overall survival (OS). Models in the hazard ratios table in FIG. 15 used binarized risk scores (at 0.67 quantile), calling one third of the samples high risk. Models in the p-values portion of the table left all risk scores continuous. All models adjusted for (T, N, Age).

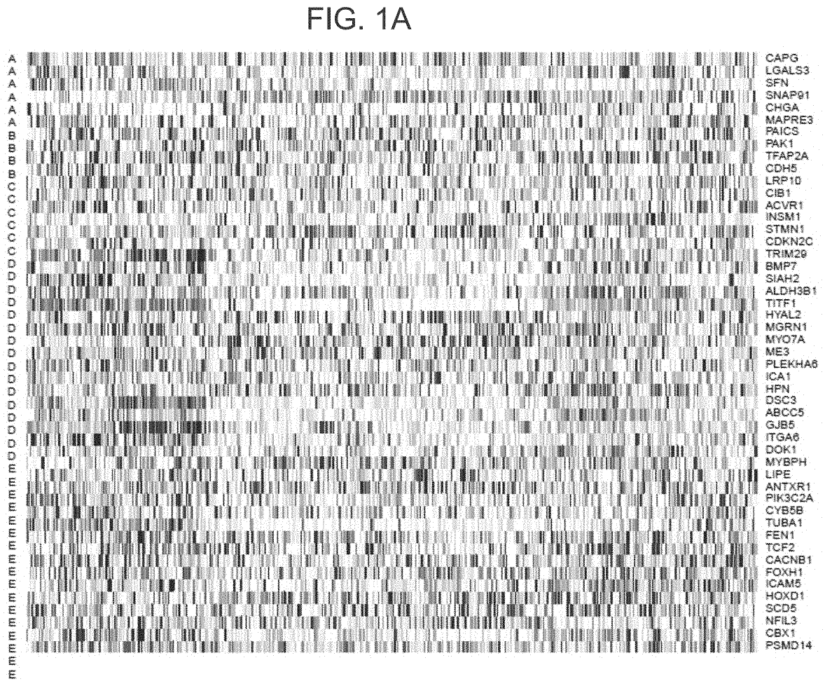

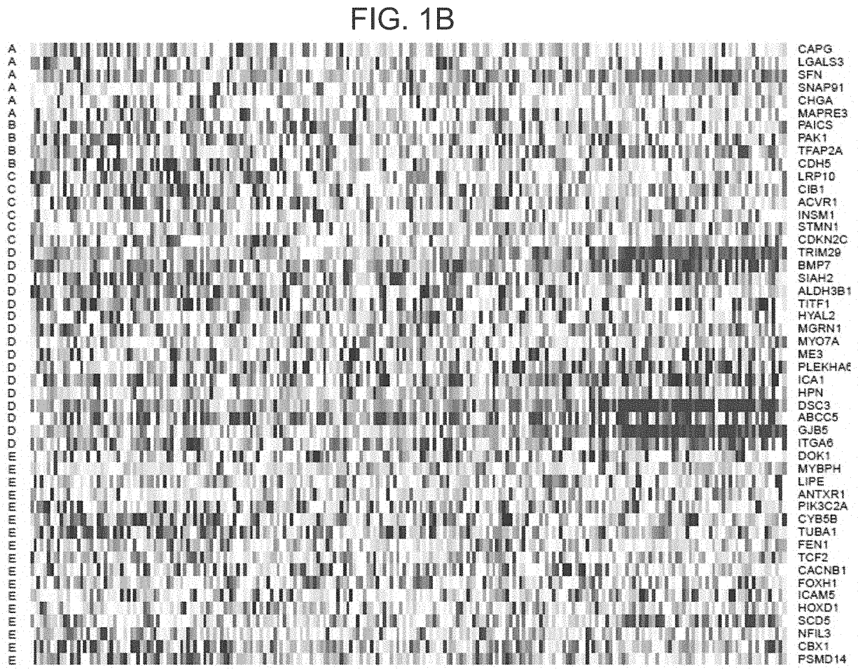

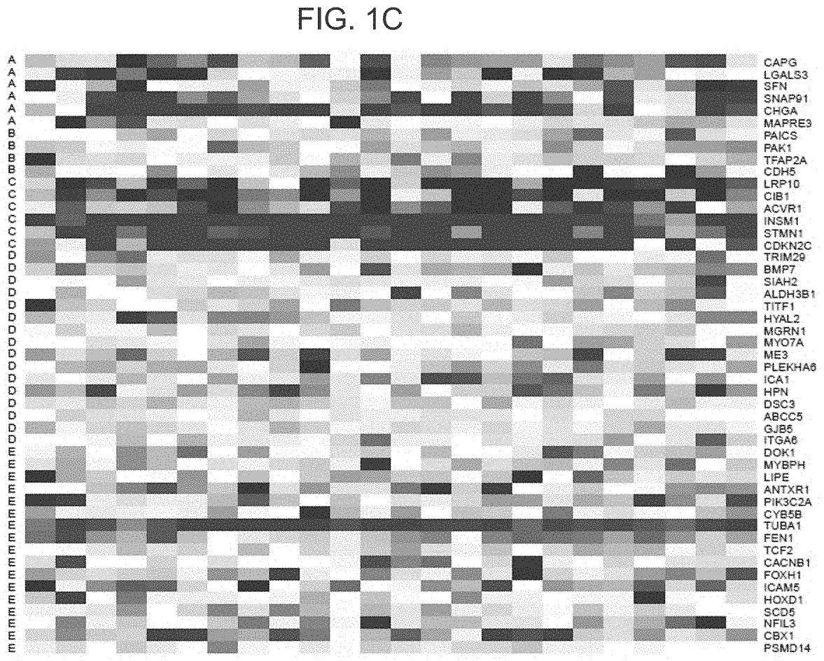

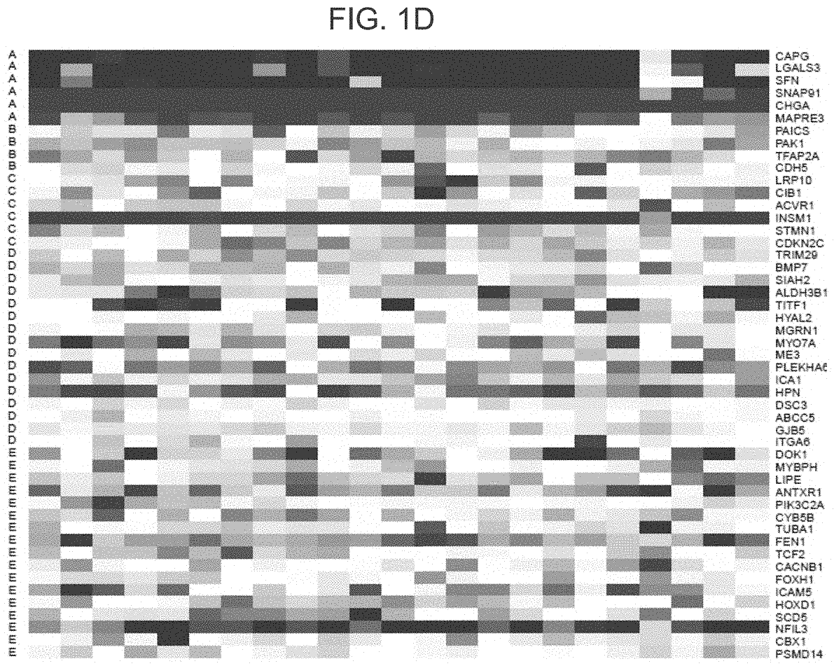

### DETAILED DESCRIPTION OF THE INVENTION

Gene expression based adenocarcinoma subtyping has been shown to classify adenocarcinoma tumors into 3 biologically distinct subtypes (Terminal Respiratory Unit (TRU; formerly referred to as Bronchioid), Proximal Inflammatory (PI; formerly referred to as Squamoid), and Proximal Proliferative (PP; formerly referred to as Magnoid)). These three subtypes vary in their prognosis, in their distribution of smokers vs. nonsmokers, in their prevalence of EGFR alterations, ALK rearrangements, TP53 mutations, and in their angiogenic features. The present invention addresses the need in the field for determining a prognosis or disease outcome for adenocarcinoma patient populations based in part on the adenocarcinoma subtype (Terminal Respiratory Unit (TRU), Proximal Inflammatory (PI), Proximal Proliferative (PP)) of the patient.

As used herein, an “expression profile” comprises one or more values corresponding to a measurement of the relative abundance, level, presence, or absence of expression of a discriminative gene. An expression profile can be derived from a subject prior to or subsequent to a diagnosis of lung cancer, can be derived from a biological sample collected from a subject at one or more time points prior to or following treatment or therapy, can be derived from a biological sample collected from a subject at one or more time points during which there is no treatment or therapy (e.g., to monitor progression of disease or to assess development of disease in a subject diagnosed with or at risk for lung cancer), or can be collected from a healthy subject. The term subject can be used interchangeably with patient. The patient can be a human patient.

As used herein, the term “determining an expression level” or “determining an expression profile” or “detecting an expression level” or “detecting an expression profile” as used in reference to a biomarker or classifier means the application of a biomarker specific reagent such as a probe, primer or antibody and/or a method to a sample, for example a sample of the subject or patient and/or a control sample, for ascertaining or measuring quantitatively, semi-quantitatively or qualitatively the amount of a biomarker or biomarkers, for example the amount of biomarker polypeptide or mRNA (or cDNA derived therefrom). For example, a level of a biomarker can be determined by a number of methods including for example immunoassays including for example immunohistochemistry, ELISA, Western blot, immunoprecipitation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR (qRT-PCR), serial analysis of gene expression (SAGE), Northern Blot, digital molecular barcoding technology, for example Nanostring Counter Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and this technology has been shown to be useful for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

The “biomarkers” or “classifier biomarkers” of the invention include genes and proteins, and variants and fragments thereof. Such biomarkers include DNA comprising the entire or partial sequence of the nucleic acid sequence encoding the biomarker, or the complement of such a sequence. The biomarker nucleic acids also include any expression product or portion thereof of the nucleic acid sequences of interest. A biomarker protein is a protein encoded by or corresponding to a DNA biomarker of the invention. A biomarker protein comprises the entire or partial amino acid sequence of any of the biomarker proteins or polypeptides.

A “biomarker” is any gene or protein whose level of expression in a tissue or cell is altered compared to that of a normal or healthy cell or tissue. The detection, and in some cases the level, of the biomarkers of the invention permits the differentiation of samples.

The biomarker panels and methods provided herein are used in various aspects, to assess, (i) whether a patient's NSCLC subtype is adenocarcinoma or squamous cell carcinoma; (ii) whether a patient's lung cancer subtype is adenocarcinoma, squamous cell carcinoma, or a neuroendocrine (encompassing both small cell carcinoma and carcinoid) and/or (iii) whether a patient's lung cancer subtype is adenocarcinoma, squamous cell carcinoma or small cell carcinoma. In one embodiment, as described herein, the methods provided herein further comprise characterizing a patient's lung cancer (adenocarcinoma) sample as proximal inflammatory (squamoid), proximal proliferative (magnoid) or terminal respiratory unit (bronchioid).

A biomarker capable of reliable classification can be one that is upregulated (e.g., expression is increased) or downregulated (e.g., expression is decreased) relative to a control. The control can be any control as provided herein. For example, the biomarker panels, or subsets thereof, as disclosed in Table 1A, Table 1B, Table 1C, Table 2, Table 3, Table 4, Table 5 and Table 6 are used in various embodiments to assess and classify a patient's lung cancer subtype.

In general, the methods provided herein are used to classify a lung cancer sample as a particular lung cancer subtype (e.g. subtype of adenocarcinoma). In one embodiment, the method comprises detecting or determining an expression level of at least five of the classifier biomarkers of Table 1A, Table 1B, Table 1C, Table 2, Table 3, Table 4, Table 5 or Table 6 in a lung cancer sample obtained from a patient or subject. In one embodiment, the detecting step is at the nucleic acid level by performing RNA-seq, a reverse transcriptase polymerase chain reaction (RT-PCR) or a hybridization assay with oligonucleotides that are substantially complementary to portions of cDNA molecules of the at least five classifier biomarkers of Table 1A, Table 1B, Table 1C, Table 2, Table 3, Table 4, Table 5 or Table 6 under conditions suitable for RNA-seq, RT-PCR or hybridization and obtaining expression levels of the at least five classifier biomarkers based on the detecting step. The expression levels of the at least five of the classifier biomarkers are then compared to reference expression levels of the at least five of the classifier biomarkers of Table 1A, Table 1B, Table 1C, Table 2, Table 3, Table 4, Table 5 or Table 6 from at least one sample training set. The at least one sample training set can comprise, (i) expression levels(s) of the at least five biomarkers from a sample that overexpresses the at least five biomarkers, or overexpresses a subset of the at least five biomarkers, (ii) expression levels from a reference squamoid (proximal inflammatory), bronchoid (terminal respiratory unit) or magnoid (proximal proliferative) sample, or (iii) expression levels from an adenocarcinoma free lung sample, and classifying the lung tissue sample as a squamoid (proximal inflammatory), bronchoid (terminal respiratory unit) or a magnoid (proximal proliferative) subtype. The lung cancer sample can then be classified as an adenocarcinoma, squamous cell carcinoma, a neuroendocrine or small cell carcinoma or even a bronchioid, squamoid, or magnoid subtype of adenocarcinoma based on the results of the comparing step. In one embodiment, the comparing step can comprise applying a statistical algorithm which comprises determining a correlation between the expression data obtained from the lung tissue or cancer sample and the expression data from the at least one training set(s); and classifying the lung tissue or cancer sample as a squamoid (proximal inflammatory), bronchoid (terminal respiratory unit) or a magnoid (proximal proliferative) subtype based on the results of the statistical algorithm.

In one embodiment, the method comprises probing the levels of at least five of the classifier biomarkers of Table 1A, Table 1B, Table 1C, Table 2, Table 3, Table 4, Table 5 or Table 6 at the nucleic acid level, in a lung cancer sample obtained from the patient. The probing step, in one embodiment, comprises mixing the sample with five or more oligonucleotides that are substantially complementary to portions of cDNA molecules of the at least five classifier biomarkers of Table 1A, Table 1B, Table 1C, Table 2, Table 3, Table 4, Table 5 or Table 6 under conditions suitable for hybridization of the five or more oligonucleotides to their complements or substantial complements; detecting whether hybridization occurs between the five or more oligonucleotides to their complements or substantial complements; and obtaining hybridization values of the at least five classifier biomarkers based on the detecting step. The hybridization values of the at least five classifier biomarkers are then compared to reference hybridization value(s) from at least one sample training set. For example, the at least one sample training set comprises hybridization values from a reference adenocarcinoma, squamous cell carcinoma, a neuroendocrine sample, small cell carcinoma sample. The lung cancer sample is classified, for example, as an adenocarcinoma, squamous cell carcinoma, a neuroendocrine or small cell carcinoma based on the results of the comparing step.

The lung tissue sample can be any sample isolated from a human subject or patient. For example, in one embodiment, the analysis is performed on lung biopsies that are embedded in paraffin wax. This aspect of the invention provides a means to improve current diagnostics by accurately identifying the major histological types, even from small biopsies. The methods of the invention, including the RT-PCR methods, are sensitive, precise and have multianalyte capability for use with paraffin embedded samples. See, for example, Cronin et al. (2004) Am. J Pathol. 164(1):35-42, herein incorporated by reference.

Formalin fixation and tissue embedding in paraffin wax is a universal approach for tissue processing prior to light microscopic evaluation. A major advantage afforded by formalin-fixed paraffin-embedded (FFPE) specimens is the preservation of cellular and architectural morphologic detail in tissue sections. (Fox et al. (1985) J Histochem Cytochem 33:845-853). The standard buffered formalin fixative in which biopsy specimens are processed is typically an aqueous solution containing 37% formaldehyde and 10-15% methyl alcohol. Formaldehyde is a highly reactive dipolar compound that results in the formation of protein-nucleic acid and protein-protein crosslinks in vitro (Clark et al. (1986) J Histochem Cytochem 34:1509-1512; McGhee and von Hippel (1975) Biochemistry 14:1281-1296, each incorporated by reference herein).

In one embodiment, the sample used herein is obtained from an individual, and comprises fresh-frozen paraffin embedded (FFPE) tissue. However, other tissue and sample types are amenable for use herein (e.g., fresh tissue, or frozen tissue).

Methods are known in the art for the isolation of RNA from FFPE tissue. In one embodiment, total RNA can be isolated from FFPE tissues as described by Bibikova et al. (2004) American Journal of Pathology 165:1799-1807, herein incorporated by reference. Likewise, the High Pure RNA Paraffin Kit (Roche) can be used. Paraffin is removed by xylene extraction followed by ethanol wash. RNA can be isolated from sectioned tissue blocks using the MasterPure Purification kit (Epicenter, Madison, Wis.); a DNase I treatment step is included. RNA can be extracted from frozen samples using Trizol reagent according to the supplier's instructions (Invitrogen Life Technologies, Carlsbad, Calif.). Samples with measurable residual genomic DNA can be resubjected to DNaseI treatment and assayed for DNA contamination. All purification, DNase treatment, and other steps can be performed according to the manufacturer's protocol. After total RNA isolation, samples can be stored at −80° C. until use.

General methods for mRNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MasterPure™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumor can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155, incorporated by reference in its entirety for all purposes).

In one embodiment, a sample comprises cells harvested from a lung tissue sample, for example, an adenocarcinoma sample. Cells can be harvested from a biological sample using standard techniques known in the art. For example, in one embodiment, cells are harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract nucleic acid, e.g, messenger RNA. All samples obtained from a subject, including those subjected to any sort of further processing, are considered to be obtained from the subject.

The sample, in one embodiment, is further processed before the detection of the biomarker levels of the combination of biomarkers set forth herein. For example, mRNA in a cell or tissue sample can be separated from other components of the sample. The sample can be concentrated and/or purified to isolate mRNA in its non-natural state, as the mRNA is not in its natural environment. For example, studies have indicated that the higher order structure of mRNA in vivo differs from the in vitro structure of the same sequence (see, e.g., Rouskin et al. (2014). Nature 505, pp. 701-705, incorporated herein in its entirety for all purposes).

mRNA from the sample in one embodiment, is hybridized to a synthetic DNA probe, which in some embodiments, includes a detection moiety (e.g., detectable label, capture sequence, barcode reporting sequence). Accordingly, in these embodiments, a non-natural mRNA-cDNA complex is ultimately made and used for detection of the biomarker. In another embodiment, mRNA from the sample is directly labeled with a detectable label, e.g., a fluorophore. In a further embodiment, the non-natural labeled-mRNA molecule is hybridized to a cDNA probe and the complex is detected.

In one embodiment, once the mRNA is obtained from a sample, it is converted to complementary DNA (cDNA) in a hybridization reaction or is used in a hybridization reaction together with one or more cDNA probes. cDNA does not exist in vivo and therefore is a non-natural molecule. Furthermore, cDNA-mRNA hybrids are synthetic and do not exist in vivo. Besides cDNA not existing in vivo, cDNA is necessarily different than mRNA, as it includes deoxyribonucleic acid and not ribonucleic acid. The cDNA is then amplified, for example, by the polymerase chain reaction (PCR) or other amplification method known to those of ordinary skill in the art. For example, other amplification methods that may be employed include the ligase chain reaction (LCR) (Wu and Wallace, Genomics, 4:560 (1989), Landegren et al., Science, 241:1077 (1988), incorporated by reference in its entirety for all purposes, transcription amplification (Kwoh et al., Proc. Natl. Acad. Sci. USA, 86:1173 (1989), incorporated by reference in its entirety for all purposes), self-sustained sequence replication (Guatelli et al., Proc. Nat. Acad. Sci. USA, 87:1874 (1990), incorporated by reference in its entirety for all purposes), incorporated by reference in its entirety for all purposes, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety for all purposes. The product of this amplification reaction, i.e., amplified cDNA is also necessarily a non-natural product. First, as mentioned above, cDNA is a non-natural molecule. Second, in the case of PCR, the amplification process serves to create hundreds of millions of cDNA copies for every individual cDNA molecule of starting material. The number of copies generated are far removed from the number of copies of mRNA that are present in vivo.

In one embodiment, cDNA is amplified with primers that introduce an additional DNA sequence (e.g., adapter, reporter, capture sequence or moiety, barcode) onto the fragments (e.g., with the use of adapter-specific primers), or mRNA or cDNA biomarker sequences are hybridized directly to a cDNA probe comprising the additional sequence (e.g., adapter, reporter, capture sequence or moiety, barcode). Amplification and/or hybridization of mRNA to a cDNA probe therefore serves to create non-natural double stranded molecules from the non-natural single stranded cDNA, or the mRNA, by introducing additional sequences and forming non-natural hybrids. Further, as known to those of ordinary skill in the art, amplification procedures have error rates associated with them. Therefore, amplification introduces further modifications into the cDNA molecules. In one embodiment, during amplification with the adapter-specific primers, a detectable label, e.g., a fluorophore, is added to single strand cDNA molecules. Amplification therefore also serves to create DNA complexes that do not occur in nature, at least because (i) cDNA does not exist in vivo, (i) adapter sequences are added to the ends of cDNA molecules to make DNA sequences that do not exist in vivo, (ii) the error rate associated with amplification further creates DNA sequences that do not exist in vivo, (iii) the disparate structure of the cDNA molecules as compared to what exists in nature and (iv) the chemical addition of a detectable label to the cDNA molecules.

In some embodiments, the expression of a biomarker of interest is detected at the nucleic acid level via detection of non-natural cDNA molecules.

In some embodiments, the method for lung cancer subtyping includes detecting expression levels of a classifier biomarker set. In some embodiments, the detecting includes all of the classifier biomarkers of Table 1 (also characterized as a lung cancer subtype gene panel), Table 2, Table 3, Table 4, Table 5 or Table 6 at the nucleic acid level or protein level. In another embodiment, a single or a subset of the classifier biomarkers of Table 1 are detected, for example, from about five to about twenty. The detecting can be performed by any suitable technique including, but not limited to, RNA-seq, a reverse transcriptase polymerase chain reaction (RT-PCR), a microarray hybridization assay, or another hybridization assay, e.g., a NanoString assay for example, with primers and/or probes specific to the classifier biomarkers, and/or the like. In some cases, the primers useful for the amplification methods (e.g., RT-PCR or qRT-PCR) are the forward and reverse primers provided in Table 1A, Table 1B, Table 1C, Table 2, Table 3, Table 4, Table 5 or Table 6. It should be noted however that the primers provided in Table 1A, Table 1B, Table 1C, Table 2, Table 3, Table 4, Table 5 and Table 6 are merely for illustrative purposes and should not be construed as limiting the invention.

The biomarkers described herein include RNA comprising the entire or partial sequence of any of the nucleic acid sequences of interest, or their non-natural cDNA product, obtained synthetically in vitro in a reverse transcription reaction. The term “fragment” is intended to refer to a portion of the polynucleotide that generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full-length biomarker polynucleotide disclosed herein. A fragment of a biomarker polynucleotide will generally encode at least 15, 25, 30, 50, 100, 150, 200, or 250 contiguous amino acids, or up to the total number of amino acids present in a full-length biomarker protein of the invention.

In some embodiments, overexpression, such as of an RNA transcript or its expression product, is determined by normalization to the level of reference RNA transcripts or their expression products, which can be all measured transcripts (or their products) in the sample or a particular reference set of RNA transcripts (or their non-natural cDNA products). Normalization is performed to correct for or normalize away both differences in the amount of RNA or cDNA assayed and variability in the quality of the RNA or cDNA used. Therefore, an assay typically measures and incorporates the expression of certain normalizing genes, including well known housekeeping genes, such as, for example, GAPDH and/or β-Actin. Alternatively, normalization can be based on the mean or median signal of all of the assayed biomarkers or a large subset thereof (global normalization approach).

For example, in one embodiment, from about 5 to about 10, from about 5 to about 15, from about 5 to about 20, from about 5 to about 25, from about 5 to about 30, from about 5 to about 35, from about 5 to about 40, from about 5 to about 45, from about 5 to about 50 of the biomarkers in any of Table 1A, Table 1B, Table 1C, Table 2, Table 3, Table 4, Table 5 and Table 6 are detected in a method to determine the lung cancer subtype. In another embodiment, each of the biomarkers from any one of Table 1A, Table 1B, Table 1C, Table 2, Table 3, Table 4, Table 5, or from Table 6 are detected in a method to determine the lung cancer subtype.

TABLE 1A

| | | | SEQ | | SEQ |
|---|---|---|---|---|---|
| Gene symbol | Gene name | Forward primer | ID | Reverse primer | ID |
| CDH5 | cadherin 5, type 2, | AAGAGAGATTG | 1 | TTCTTGCGACTCACGCT | 58 |
| | VE-cadherin | GATTTGGAACC | | | |
| | (vascular epithelium) | | | | |
| CLEC3B | C-type lectin domain | CCAGAAGCCCA | 2 | GCTCCTCAAACAT | 59 |
| | family 3, member B | AGAAGATTGTA | | CTTTGTGTTCA | |
| PAICS | phosphoribosylami | AATCCTGGTGT | 3 | GACCACTGTGGG | 60 |
| | noimidazole | CAAGGAAG | | TCATTATT | |
| | carboxylase, | | | | |
| | phosphoribosylami | | | | |
| | noimidazole | | | | |
| | succinocarboxamide | | | | |
| | synthetase | | | | |
| PAK1 | p21/Cdc42/Rac1- | GGACCGATTTT | 4 | GAAATCTCTGGC | 61 |
| | activated kinase 1 (STE20 | ACCGATCC | | CGCTC | |
| | homolog, yeast) | | | | |
| PECAM1 | platelet/endothelial cell | ACAGTCCAGAT | 5 | ACTGGGCATCAT | 62 |
| | adhesion molecule | AGTCGTATGT | | AAGAAATCC | |
| | (CD31 antigen) | | | | |
| TFAP2A | transcription factor AP- | GTCTCCGCCATC | 6 | ACTGAACAGAAG | 63 |
| | 2 alpha (activating | CCTAT | | ACATTCGT | |
| | enhancer binding | | | | |
| | protein 2 alpha) | | | | |
| ACVR1 | activin A receptor, | ACTGGTGTAAC | 7 | AACCTCCAAGTG | 64 |
| | type 1 | AGGAACAT | | GAAATTCT | |
| CDKN2C | cyclin-dependent kinase | TTTGGAAGGAC | 8 | TCGGTCTTTCAAA | 65 |
| | inhibitor 2C (p18, | TGCGCT | | TCGGGATTA | |
| | inhibits CDK4) | | | | |
| CIB1 | calcium and integrin | CACGTCATCTCC | 9 | CTGCTGTCACAG | 66 |
| | binding 1 (calmyrin) | CGTTC | | GACAAT | 66 |
| INSM1 | insulinoma-associated 1 | ATTGAACTTCCC | 10 | AAGGTAAAGCCA | 67 |
| | | ACACGA | | GACTCCA | 67 |
| ERP10 | low density lipoprotein | GGAACAGACTG | 11 | GGGAGCGTAGGG | 68 |
| | receptor-related protein | TCACCAT | | TTAAG | |
| | 10 | | | | |
| STMN1 | stathmin | TCAGAGTGTGTG | 12 | CAGTGTATTCTGC | 69 |
| | 1/oncoprotein 18 | G | | ACAATCAAC | |
| | | TCAGGC | | | |
| CAPG | capping protein (actin | GGGACAGCTTC | 13 | GTTCCAGGATGTT | 70 |
| | filament), gelsolin-like | AACACT | | GGACTTTC | |
| CHGA | chromogranin A | CCTGTGAACAG | 14 | GGAAAGTGTGTC | 71 |
| | (parathyroid secretory | CCCTATG | | GGAGAT | |
| | protein 1) | | | | |
| LGALS3 | lectin, galactoside- | TTCTGGGCACG | 15 | AGGCAACATCAT | 72 |
| | binding, soluble, 3 | GTGAAG | | TCCCTC | |
| | (galectin 3) | | | | |
| MAPRE3 | microtubule-associated | GGCCAAACTAG | 16 | GTCAACACCCAT | 73 |
| | protein, RP/EB family, | AGCACGAATA | | CTTCATTGAAA | |
| | member 3 | | | | |
| SFN | stratifin | TCAGCAAGAAG | 17 | CGTAGTGGAAGA | 74 |
| | | GAGATGCC | | CGGAAA | |
| SNAP91 | synaptosomal-associated | GTGCTCCCTCTC | 18 | CTGGTGTAGAATT | 75 |
| | protein, 91 kDa | CATTAAGTA | | AGGAGACGTA | |
| | homolog (mouse) | | | | |
| ABCC5 | ATP-binding cassette, | CAAGTTCAGGA | 19 | GGCATCAAGAGA | 76 |
| | sub-family C (CFTR/MRP), | GAACTCGAC | | GAGGC | |
| | member 5 | | | | |
| ALDH3B1 | aldehyde dehydrogenase | GGCTGTGGTTA | 20 | GATAAAGAGTTA | 77 |
| | 3 | TGCGATAG | | CAAGCTCCTCIG | |
| | family, member B1 | | | | |
| ANTXR1 | Anthrax toxin receptor 1 | ACCCGAGGAAC | 21 | TCTAGGCCTTGAC | 78 |
| | | AACCTTA | | GGAT | |
| BMP7 | Bone morphogenetic | CCCTCTCCATTCC | 22 | TTTGGGCAAACCTCGGTA | 79 |
| | protein 7 (osteogenic | CTACA | | A | |
| | protein 1) | | | | |
| CACNB1 | calcium channel, | CAGAGCGCCAG | 23 | GCACAGCAAATG | 80 |
| | voltage-dependent, beta | GCATTA | | CCACT | |
| | 1 subunit | | | | |
| CBX1 | chromobox homolog 1 | CCACTGGCTGA | 24 | CTTGTCTTTCCCT | 81 |
| | (HP1 bent homolog | GGTGTTA | | ACTGTCTTAC | |
| | Drosophila) | | | | |
| CYB5B | cytochrome b5 type B | TGGGCGAGTCT | 25 | CTTGTTCCAGCAG | 82 |
| | (outer mitochondrial | ACGATG | | AACCT | |
| | membrane) | | | | |
| DOK1 | docking protein 1, 62 | CTTTCTGCCCTG | 26 | CAGTCCTCTGCAC | 83 |
| | kDa (downstream of | GAGATG | | CGTTA | |
| | tyrosine kinase 1) | | | | |
| DSC3 | desmocollin 3 | GCGCCATTTGCT | 27 | CATCCAGATCCCT | 84 |
| | | AGAGATA | | CACAT | |
| FEN1 | flap structure-specific | AGAGAAGATGG | 28 | CCAAGACACAGC | 85 |
| | endonuclease 1 | GCAGAAAG | | CAGTAAT | |
| FOXH1 | forkhead box H1 | GCCCAGATCAT | 29 | TTTCCAGCCCTCG | 86 |
| | | CCGTCA | | TAGTC | |
| GJB5 | gap junction protein, | ACCACAAGGAC | 30 | GGGACACAGGGA | 87 |
| | beta 5 (connexin 31.1) | TTCGAC | | AGAAC | |
| HOXD1 | homeobox D1 | GCTCCGCTGCT | 31 | GTCTGCCACTCTG | 88 |
| | | ATCTTT | | CAAC | |
| HPN | Hepsin (transmembrane | AGCGGCCAGGT | 32 | GTCGGCTGACGC | 89 |
| | protease, serine 1) | GGATTA | | TTTGA | |
| HYAL2 | hyaluronoglucosam | ATGGGCTTTGG | 33 | GAACAAGTCAGT | 90 |
| | inidase 2 | GAGCATA | | CTAGGGAATAC | |
| ICA1 | islet cell autoantigen | GACCTGGATGC | 34 | TGCTTTCGATAAG | 91 |
| | 1, 69 kDa | CAAGCTA | | TCCAGACA | |
| ICAM5 | intercellular adhesion | CCGGCTCTTGG | 35 | CCTCTGAGGCTG | 92 |
| | molecule 5, | AAGTTG | | GAAACA | |
| | telencephalin | | | | |
| ITGA6 | integrin, alpha 6 | ACGCGGATCGA | 36 | ATCCACTGATCTT | 93 |
| | | GTTTGATAA | | CCTTGC | |
| LIPE | lipase, hormone-sensitive | CGCAAGTCCCA | 37 | CAGTGCTGCTTCA | 94 |
| | | GAAGAT | | GACACA | |
| ME3 | malic enzyme 3, | CGCGGATACGA | 38 | CCTTTCTTCAAGG | 95 |
| | NADP(+)-dependent, | TGTCAC | | GTAAAGGC | |
| | Mitochondrial | | | | |
| MGRN1 | mahogunin, ring finger | GAACTCGGCCT | 39 | TCGAATTTCTCTC | 96 |
| | 1 | ATCGCT | | CTCCCAT | |
| MYBPH | myosin binding protein | TCTGACCTCATC | 40 | CTGAGTCCACAC | 97 |
| | H | ATCGGCAA | | AGGTTT | |
| MYO7A | myosin VIIA | GAGGTGAAGCA | 41 | CCCATACTTGTTG | 98 |
| | | AACTACGGA | | ATGGCAATTA | |
| NFIL3 | nuclear factor, | ACTCTCCACAA | 42 | TCCTGCGTGTGTT | 99 |
| | interleukin 3 regulated | AGCTCG | | CTACT | |
| PIK3C2A | phosphoinositide-3-kinase | GGATTTCAGCT | 43 | AGTCATCATGTAC | 100 |
| | class 2, alpha | ACCAGTTACTT | | CCAGCA | |
| | polypeptide | | | | |
| PLEKHA6 | pleckstrin homology | TTCGTCCTGGTG | 44 | CCCAGGATACTCT | 101 |
| | domain containing, | GATCG | | CTTCCTT | |
| | family A member 6 | | | | |
| PSMD14 | proteasome (prosome, | AGTGATTGATG | 45 | CACTGGATCAAC | 102 |
| | macropain) 26S subunit, | TGTTTGCTATG | | TGCCTC | |
| | non-ATPase, 14 | | | | |
| SCD5 | stearoyk-CoA desaturase | CAAAGCCAAGC | 46 | CAGCTGTCACAC | 103 |
| | 5 | CACTCACTC | | CCAGAGC | |
| S1AH2 | seven in absentia | CTCGGCAGTCC | 47 | CGTATGGTGCAG | 104 |
| | homolog 2 | TGTTTC | | GGTCA | |
| | (Drosophila) | | | | |
| TCF2 | transcription factor 2, | ACACCTGGTAC | 48 | TCTGGACTGTCTG | 105 |
| | hepatic; LF-B3; variant | GTCACAA | | GTTGAAT | |
| | hepatic nuclear factor | | | | |
| TCP1 | t-complex 1 | ATGCCCAAGAG | 49 | CCTGTACACCAA | 106 |
| | | AATCGTAAA | | GCTTCAT | |
| TTF1 | thyroid transcription | ATGAGTCCAAA | 50 | CCATGCCCACTTT | 107 |
| | factor 1 | GCACACGA | | CTTGTA | |
| TRIM29 | tripartite motif-containing | TGAGATTGAGG | 51 | CATTGGTGGTGA | 108 |
| | 29 | ATGAAGCTGAG | | AGCTCTTG | |
| TUBA1 | tubulin, alpha 1 | CCGACTCAACG | 52 | CGTGGACTGAGA | 109 |
| | | TGAGAC | | TGCATT | |
| CFL1 | cofilin 1 (non-muscle) | GTGCCCTCTCCT | 53 | TTCATGTCGTTGA | 110 |
| | | TTTCG | | ACACCTTG | |
| EEF1A1 | eukaryotic translation | CGTTCTTTTTCG | 54 | CATTTTGGCTTTT | 111 |
| | elongation factor 1 | CAACGG | | AGGGGTAG | |
| | alpha 1 | | | | |
| RPL10 | ribosomal protein L10 | GGTGTGCCACT | 55 | GGCAGAAGCGAG | 112 |
| | | GAAGAT | | ACTTT | |
| RPL28 | ribosomal protein L28 | GTGTCGTGGTG | 56 | GCACATAGGAGG | 113 |
| | | GTCATT | | TGGCA | |
| RPL37A | ribosomal protein L37a | GCATGAAGACA | 57 | GCGGACTTTACC | 114 |
| | | GTGGCT | | GTGAC | |

TABLE 1B

| | | | SEQ | | SEQ |
|---|---|---|---|---|---|
| Gene symbol | Gene name | Forward primer | ID | Reverse primer | ID |
| CDH5 | cadherin 5, type 2, | AAGAGAGATTG | 1 | TTCTTGCGACTCACGCT | 58 |
| | VE-cadherin | GATTTGGAACC | | | |
| | (vascular epithelium) | | | | |
| CLEC3B | C-type lectin domain | CCAGAAGCCCA | 2 | GCTCCTCAAACAT | 59 |
| | family 3, member B | AGAAGATTGTA | | CTTTGTGTTCA | |
| PAICS | phosphoribosylami | AATCCTGGTGT | 3 | GACCACTGTGGG | 60 |
| | noimidazole | CAAGGAAG | | TCATTATT | |
| | carboxylase, | | | | |
| | phosphoribosylami | | | | |
| | noimidazole | | | | |
| | succinocarboxamide | | | | |
| | synthetase | | | | |
| PAK1 | p21/Cdc42/Rac1- | GGACCGATTTT | 4 | GAAATCTCTGGC | 61 |
| | activated kinase 1 (STE20 | ACCGATCC | | CGCTC | |
| | homolog, yeast) | | | | |
| PECAM1 | platelet/endothelial cell | ACAGTCCAGAT | 5 | ACTGGGCATCAT | 62 |
| | adhesion molecule | AGTCGTATGT | | AAGAAATCC | |
| | (CD31 antigen) | | | | |
| TFAP2A | transcription factor AP- | GTCTCCGCCATC | 6 | ACTGAACAGAAG | 63 |
| | 2 alpha (activating | CCTAT | | ACATTCGT | |
| | enhancer binding | | | | |
| | protein 2 alpha) | | | | |
| ACVR1 | activin A receptor, | ACTGGTGTAAC | 7 | AACCTCCAAGTG | 64 |
| | type 1 | AGGAACAT | | GAAATTCT | |
| CDKN2C | cyclin-dependent kinase | TTTGGAAGGAC | 8 | TCGGTCTTTCAAA | 65 |
| | inhibitor 2C (p18, | TGCGCT | | TCGGGATTA | |
| | inhibits CDK4) | | | | |
| CIB1 | calcium and integrin | CACGTCATCTCC | 9 | CTGCTGTCACAG | 66 |
| | binding 1 (calmyrin) | CGTTC | | GACAAT | 66 |
| INSM1 | insulinoma-associated 1 | ATTGAACTTCCC | 10 | AAGGTAAAGCCA | 67 |
| | | ACACGA | | GACTCCA | 67 |
| ERP10 | low density lipoprotein | GGAACAGACTG | 11 | GGGAGCGTAGGG | 68 |
| | receptor-related protein | TCACCAT | | TTAAG | |
| | 10 | | | | |
| STMN1 | stathmin | TCAGAGTGTGTG | 12 | CAGTGTATTCTGC | 69 |
| | 1/oncoprotein 18 | G | | ACAATCAAC | |
| | | TCAGGC | | | |
| CAPG | capping protein (actin | GGGACAGCTTC | 13 | GTTCCAGGATGTT | 70 |
| | filament), gelsolin-like | AACACT | | GGACTTTC | |
| CHGA | chromogranin A | CCTGTGAACAG | 14 | GGAAAGTGTGTC | 71 |
| | (parathyroid secretory | CCCTATG | | GGAGAT | |
| | protein 1) | | | | |
| LGALS3 | lectin, galactoside- | TTCTGGGCACG | 15 | AGGCAACATCAT | 72 |
| | binding, soluble, 3 | GTGAAG | | TCCCTC | |
| | (galectin 3) | | | | |
| MAPRE3 | microtubule-associated | GGCCAAACTAG | 16 | GTCAACACCCAT | 73 |
| | protein, RP/EB family, | AGCACGAATA | | CTTCATTGAAA | |
| | member 3 | | | | |
| SFN | stratifin | TCAGCAAGAAG | 17 | CGTAGTGGAAGA | 74 |
| | | GAGATGCC | | CGGAAA | |
| SNAP91 | synaptosomal-associated | GTGCTCCCTCTC | 18 | CTGGTGTAGAATT | 75 |
| | protein, 91 kDa | CATTAAGTA | | AGGAGACGTA | |
| | homolog (mouse) | | | | |
| ABCC5 | ATP-binding cassette, | CAAGTTCAGGA | 19 | GGCATCAAGAGA | 76 |
| | sub-family C (CFTR/MRP), | GAACTCGAC | | GAGGC | |
| | member 5 | | | | |
| ALDH3B1 | aldehyde dehydrogenase | GGCTGTGGTTA | 20 | GATAAAGAGTTA | 77 |
| | 3 | TGCGATAG | | CAAGCTCCTCIG | |
| | family, member B1 | | | | |
| ANTXR1 | Anthrax toxin receptor 1 | ACCCGAGGAAC | 21 | TCTAGGCCTTGAC | 78 |
| | | AACCTTA | | GGAT | |
| CACNB1 | calcium channel, | CAGAGCGCCAG | 23 | GCACAGCAAATG | 80 |
| | voltage-dependent, beta | GCATTA | | CCACT | |
| | 1 subunit | | | | |
| CBX1 | chromobox homolog 1 | CCACTGGCTGA | 24 | CTTGTCTTTCCCT | 81 |
| | (HP1 bent homolog | GGTGTTA | | ACTGTCTTAC | |
| | Drosophila) | | | | |
| CYB5B | cytochrome b5 type B | TGGGCGAGTCT | 25 | CTTGTTCCAGCAG | 82 |
| | (outer mitochondrial | ACGATG | | AACCT | |
| | membrane) | | | | |
| DOK1 | docking protein 1, 62 | CTTTCTGCCCTG | 26 | CAGTCCTCTGCAC | 83 |
| | kDa (downstream of | GAGATG | | CGTTA | |
| | tyrosine kinase 1) | | | | |
| DSC3 | desmocollin 3 | GCGCCATTTGCT | 27 | CATCCAGATCCCT | 84 |
| | | AGAGATA | | CACAT | |
| FEN1 | flap structure-specific | AGAGAAGATGG | 28 | CCAAGACACAGC | 85 |
| | endonuclease 1 | GCAGAAAG | | CAGTAAT | |
| FOXH1 | forkhead box H1 | GCCCAGATCAT | 29 | TTTCCAGCCCTCG | 86 |
| | | CCGTCA | | TAGTC | |
| GJB5 | gap junction protein, | ACCACAAGGAC | 30 | GGGACACAGGGA | 87 |
| | beta 5 (connexin 31.1) | TTCGAC | | AGAAC | |
| HOXD1 | homeobox D1 | GCTCCGCTGCT | 31 | GTCTGCCACTCTG | 88 |
| | | ATCTTT | | CAAC | |
| HPN | Hepsin (transmembrane | AGCGGCCAGGT | 32 | GTCGGCTGACGC | 89 |
| | protease, serine 1) | GGATTA | | TTTGA | |
| HYAL2 | hyaluronoglucosam | ATGGGCTTTGG | 33 | GAACAAGTCAGT | 90 |
| | inidase 2 | GAGCATA | | CTAGGGAATAC | |
| ICA1 | islet cell autoantigen | GACCTGGATGC | 34 | TGCTTTCGATAAG | 91 |
| | 1, 69 kDa | CAAGCTA | | TCCAGACA | |
| ICAM5 | intercellular adhesion | CCGGCTCTTGG | 35 | CCTCTGAGGCTG | 92 |
| | molecule 5, | AAGTTG | | GAAACA | |
| | telencephalin | | | | |
| ITGA6 | integrin, alpha 6 | ACGCGGATCGA | 36 | ATCCACTGATCTT | 93 |
| | | GTTTGATAA | | CCTTGC | |
| LIPE | lipase, hormone-sensitive | CGCAAGTCCCA | 37 | CAGTGCTGCTTCA | 94 |
| | | GAAGAT | | GACACA | |
| ME3 | malic enzyme 3, | CGCGGATACGA | 38 | CCTTTCTTCAAGG | 95 |
| | NADP(+)-dependent, | TGTCAC | | GTAAAGGC | |
| | Mitochondrial | | | | |
| MGRN1 | mahogunin, ring finger | GAACTCGGCCT | 39 | TCGAATTTCTCTC | 96 |
| | 1 | ATCGCT | | CTCCCAT | |
| MYBPH | myosin binding protein | TCTGACCTCATC | 40 | CTGAGTCCACAC | 97 |
| | H | ATCGGCAA | | AGGTTT | |
| MYO7A | myosin VIIA | GAGGTGAAGCA | 41 | CCCATACTTGTTG | 98 |
| | | AACTACGGA | | ATGGCAATTA | |
| NFIL3 | nuclear factor, | ACTCTCCACAA | 42 | TCCTGCGTGTGTT | 99 |
| | interleukin 3 regulated | AGCTCG | | CTACT | |
| PIK3C2A | phosphoinositide-3-kinase | GGATTTCAGCT | 43 | AGTCATCATGTAC | 100 |
| | class 2, alpha | ACCAGTTACTT | | CCAGCA | |
| | polypeptide | | | | |
| PLEKHA6 | pleckstrin homology | TTCGTCCTGGTG | 44 | CCCAGGATACTCT | 101 |
| | domain containing, | GATCG | | CTTCCTT | |
| | family A member 6 | | | | |
| PSMD14 | proteasome (prosome, | AGTGATTGATG | 45 | CACTGGATCAAC | 102 |
| | macropain) 26S subunit, | TGTTTGCTATG | | TGCCTC | |
| | non-ATPase, 14 | | | | |
| SCD5 | stearoyk-CoA desaturase | CAAAGCCAAGC | 46 | CAGCTGTCACAC | 103 |
| | 5 | CACTCACTC | | CCAGAGC | |
| S1AH2 | seven in absentia | CTCGGCAGTCC | 47 | CGTATGGTGCAG | 104 |
| | homolog 2 | TGTTTC | | GGTCA | |
| | (Drosophila) | | | | |
| TCF2 | transcription factor 2, | ACACCTGGTAC | 48 | TCTGGACTGTCTG | 105 |
| | hepatic; LF-B3; variant | GTCACAA | | GTTGAAT | |
| | hepatic nuclear factor | | | | |
| TCP1 | t-complex 1 | ATGCCCAAGAG | 49 | CCTGTACACCAA | 106 |
| | | AATCGTAAA | | GCTTCAT | |
| TTF1 | thyroid transcription | ATGAGTCCAAA | 50 | CCATGCCCACTTT | 107 |
| | factor 1 | GCACACGA | | CTTGTA | |
| TRIM29 | tripartite motif-containing | TGAGATTGAGG | 51 | CATTGGTGGTGA | 108 |
| | 29 | ATGAAGCTGAG | | AGCTCTTG | |
| TUBA1 | tubulin, alpha 1 | CCGACTCAACG | 52 | CGTGGACTGAGA | 109 |
| | | TGAGAC | | TGCATT | |
| CFL1 | cofilin 1 (non-muscle) | GTGCCCTCTCCT | 53 | TTCATGTCGTTGA | 110 |
| | | TTTCG | | ACACCTTG | |
| EEF1A1 | eukaryotic translation | CGTTCTTTTTCG | 54 | CATTTTGGCTTTT | 111 |
| | elongation factor 1 | CAACGG | | AGGGGTAG | |
| | alpha 1 | | | | |
| RPL10 | ribosomal protein L10 | GGTGTGCCACT | 55 | GGCAGAAGCGAG | 112 |
| | | GAAGAT | | ACTTT | |
| RPL28 | ribosomal protein L28 | GTGTCGTGGTG | 56 | GCACATAGGAGG | 113 |
| | | GTCATT | | TGGCA | |
| RPL37A | ribosomal protein L37a | GCATGAAGACA | 57 | GCGGACTTTACC | 114 |
| | | GTGGCT | | GTGAC | |

TABLE 1C

| | | | SEQ | | SEQ |
|---|---|---|---|---|---|
| Gene symbol | Gene name | Forward primer | ID | Reverse primer | ID |
| CDH5 | cadherin 5, type 2, | AAGAGAGATTG | 1 | TTCTTGCGACTCACGCT | 58 |
| | VE-cadherin | GATTTGGAACC | | | |
| | (vascular epithelium) | | | | |
| CLEC3B | C-type lectin domain | CCAGAAGCCCA | 2 | GCTCCTCAAACAT | 59 |
| | family 3, member B | AGAAGATTGTA | | CTTTGTGTTCA | |
| PAICS | phosphoribosylami | AATCCTGGTGT | 3 | GACCACTGTGGG | 60 |
| | noimidazole | CAAGGAAG | | TCATTATT | |
| | carboxylase, | | | | |
| | phosphoribosylami | | | | |
| | noimidazole | | | | |
| | succinocarboxamide | | | | |
| | synthetase | | | | |
| PAK1 | p21/Cdc42/Rac1- | GGACCGATTTT | 4 | GAAATCTCTGGC | 61 |
| | activated kinase 1 (STE20 | ACCGATCC | | CGCTC | |
| | homolog, yeast) | | | | |
| PECAM1 | platelet/endothelial cell | ACAGTCCAGAT | 5 | ACTGGGCATCAT | 62 |
| | adhesion molecule | AGTCGTATGT | | AAGAAATCC | |
| | (CD31 antigen) | | | | |
| TFAP2A | transcription factor AP- | GTCTCCGCCATC | 6 | ACTGAACAGAAG | 63 |
| | 2 alpha (activating | CCTAT | | ACATTCGT | |
| | enhancer binding | | | | |
| | protein 2 alpha) | | | | |
| ACVR1 | activin A receptor, | ACTGGTGTAAC | 7 | AACCTCCAAGTG | 64 |
| | type 1 | AGGAACAT | | GAAATTCT | |
| CDKN2C | cyclin-dependent kinase | TTTGGAAGGAC | 8 | TCGGTCTTTCAAA | 65 |
| | inhibitor 2C (p18, | TGCGCT | | TCGGGATTA | |
| | inhibits CDK4) | | | | |
| CIB1 | calcium and integrin | CACGTCATCTCC | 9 | CTGCTGTCACAG | 66 |
| | binding 1 (calmyrin) | CGTTC | | GACAAT | 66 |
| INSM1 | insulinoma-associated 1 | ATTGAACTTCCC | 10 | AAGGTAAAGCCA | 67 |
| | | ACACGA | | GACTCCA | 67 |
| ERP10 | low density lipoprotein | GGAACAGACTG | 11 | GGGAGCGTAGGG | 68 |
| | receptor-related protein | TCACCAT | | TTAAG | |
| | 10 | | | | |
| STMN1 | stathmin | TCAGAGTGTGTG | 12 | CAGTGTATTCTGC | 69 |
| | 1/oncoprotein 18 | G | | ACAATCAAC | |
| | | TCAGGC | | | |
| CAPG | capping protein (actin | GGGACAGCTTC | 13 | GTTCCAGGATGTT | 70 |
| | filament), gelsolin-like | AACACT | | GGACTTTC | |
| CHGA | chromogranin A | CCTGTGAACAG | 14 | GGAAAGTGTGTC | 71 |
| | (parathyroid secretory | CCCTATG | | GGAGAT | |
| | protein 1) | | | | |
| LGALS3 | lectin, galactoside- | TTCTGGGCACG | 15 | AGGCAACATCAT | 72 |
| | binding, soluble, 3 | GTGAAG | | TCCCTC | |
| | (galectin 3) | | | | |
| MAPRE3 | microtubule-associated | GGCCAAACTAG | 16 | GTCAACACCCAT | 73 |
| | protein, RP/EB family, | AGCACGAATA | | CTTCATTGAAA | |
| | member 3 | | | | |
| SFN | stratifin | TCAGCAAGAAG | 17 | CGTAGTGGAAGA | 74 |
| | | GAGATGCC | | CGGAAA | |
| SNAP91 | synaptosomal-associated | GTGCTCCCTCTC | 18 | CTGGTGTAGAATT | 75 |
| | protein, 91 kDa | CATTAAGTA | | AGGAGACGTA | |
| | homolog (mouse) | | | | |
| ABCC5 | ATP-binding cassette, | CAAGTTCAGGA | 19 | GGCATCAAGAGA | 76 |
| | sub-family C (CFTR/MRP), | GAACTCGAC | | GAGGC | |
| | member 5 | | | | |
| ALDH3B1 | aldehyde dehydrogenase | GGCTGTGGTTA | 20 | GATAAAGAGTTA | 77 |
| | 3 | TGCGATAG | | CAAGCTCCTCIG | |
| | family, member B1 | | | | |
| ANTXR1 | Anthrax toxin receptor 1 | ACCCGAGGAAC | 21 | TCTAGGCCTTGAC | 78 |
| | | AACCTTA | | GGAT | |
| BMP7 | Bone morphogenetic | CCCTCTCCATTCC | 22 | TTTGGGCAAACCTCGGTA | 79 |
| | protein 7 (osteogenic | CTACA | | A | |
| | protein 1) | | | | |
| CACNB1 | calcium channel, | CAGAGCGCCAG | 23 | GCACAGCAAATG | 80 |
| | voltage-dependent, beta | GCATTA | | CCACT | |
| | 1 subunit | | | | |
| CBX1 | chromobox homolog 1 | CCACTGGCTGA | 24 | CTTGTCTTTCCCT | 81 |
| | (HP1 bent homolog | GGTGTTA | | ACTGTCTTAC | |
| | Drosophila) | | | | |
| CYB5B | cytochrome b5 type B | TGGGCGAGTCT | 25 | CTTGTTCCAGCAG | 82 |
| | (outer mitochondrial | ACGATG | | AACCT | |
| | membrane) | | | | |
| DOK1 | docking protein 1, 62 | CTTTCTGCCCTG | 26 | CAGTCCTCTGCAC | 83 |
| | kDa (downstream of | GAGATG | | CGTTA | |
| | tyrosine kinase 1) | | | | |
| DSC3 | desmocollin 3 | GCGCCATTTGCT | 27 | CATCCAGATCCCT | 84 |
| | | AGAGATA | | CACAT | |
| FEN1 | flap structure-specific | AGAGAAGATGG | 28 | CCAAGACACAGC | 85 |
| | endonuclease 1 | GCAGAAAG | | CAGTAAT | |
| FOXH1 | forkhead box H1 | GCCCAGATCAT | 29 | TTTCCAGCCCTCG | 86 |
| | | CCGTCA | | TAGTC | |
| GJB5 | gap junction protein, | ACCACAAGGAC | 30 | GGGACACAGGGA | 87 |
| | beta 5 (connexin 31.1) | TTCGAC | | AGAAC | |
| HOXD1 | homeobox D1 | GCTCCGCTGCT | 31 | GTCTGCCACTCTG | 88 |
| | | ATCTTT | | CAAC | |
| HPN | Hepsin (transmembrane | AGCGGCCAGGT | 32 | GTCGGCTGACGC | 89 |
| | protease, serine 1) | GGATTA | | TTTGA | |
| HYAL2 | hyaluronoglucosam | ATGGGCTTTGG | 33 | GAACAAGTCAGT | 90 |
| | inidase 2 | GAGCATA | | CTAGGGAATAC | |
| ICA1 | islet cell autoantigen | GACCTGGATGC | 34 | TGCTTTCGATAAG | 91 |
| | 1, 69 kDa | CAAGCTA | | TCCAGACA | |
| ICAM5 | intercellular adhesion | CCGGCTCTTGG | 35 | CCTCTGAGGCTG | 92 |
| | molecule 5, | AAGTTG | | GAAACA | |
| | telencephalin | | | | |
| ITGA6 | integrin, alpha 6 | ACGCGGATCGA | 36 | ATCCACTGATCTT | 93 |
| | | GTTTGATAA | | CCTTGC | |
| LIPE | lipase, hormone-sensitive | CGCAAGTCCCA | 37 | CAGTGCTGCTTCA | 94 |
| | | GAAGAT | | GACACA | |
| ME3 | malic enzyme 3, | CGCGGATACGA | 38 | CCTTTCTTCAAGG | 95 |
| | NADP(+)-dependent, | TGTCAC | | GTAAAGGC | |
| | Mitochondrial | | | | |
| MGRN1 | mahogunin, ring finger | GAACTCGGCCT | 39 | TCGAATTTCTCTC | 96 |
| | 1 | ATCGCT | | CTCCCAT | |
| MYBPH | myosin binding protein | TCTGACCTCATC | 40 | CTGAGTCCACAC | 97 |
| | H | ATCGGCAA | | AGGTTT | |
| MYO7A | myosin VIIA | GAGGTGAAGCA | 41 | CCCATACTTGTTG | 98 |
| | | AACTACGGA | | ATGGCAATTA | |
| NFIL3 | nuclear factor, | ACTCTCCACAA | 42 | TCCTGCGTGTGTT | 99 |
| | interleukin 3 regulated | AGCTCG | | CTACT | |
| PIK3C2A | phosphoinositide-3-kinase | GGATTTCAGCT | 43 | AGTCATCATGTAC | 100 |
| | class 2, alpha | ACCAGTTACTT | | CCAGCA | |
| | polypeptide | | | | |
| PLEKHA6 | pleckstrin homology | TTCGTCCTGGTG | 44 | CCCAGGATACTCT | 101 |
| | domain containing, | GATCG | | CTTCCTT | |
| | family A member 6 | | | | |
| PSMD14 | proteasome (prosome, | AGTGATTGATG | 45 | CACTGGATCAAC | 102 |
| | macropain) 26S subunit, | TGTTTGCTATG | | TGCCTC | |
| | non-ATPase, 14 | | | | |
| SCD5 | stearoyk-CoA desaturase | CAAAGCCAAGC | 46 | CAGCTGTCACAC | 103 |
| | 5 | CACTCACTC | | CCAGAGC | |
| S1AH2 | seven in absentia | CTCGGCAGTCC | 47 | CGTATGGTGCAG | 104 |
| | homolog 2 | TGTTTC | | GGTCA | |
| | (Drosophila) | | | | |
| TCF2 | transcription factor 2, | ACACCTGGTAC | 48 | TCTGGACTGTCTG | 105 |
| | hepatic; LF-B3; variant | GTCACAA | | GTTGAAT | |
| | hepatic nuclear factor | | | | |
| TCP1 | t-complex 1 | ATGCCCAAGAG | 49 | CCTGTACACCAA | 106 |
| | | AATCGTAAA | | GCTTCAT | |
| TTF1 | thyroid transcription | ATGAGTCCAAA | 50 | CCATGCCCACTTT | 107 |
| | factor 1 | GCACACGA | | CTTGTA | |
| TRIM29 | tripartite motif-containing | TGAGATTGAGG | 51 | CATTGGTGGTGA | 108 |
| | 29 | ATGAAGCTGAG | | AGCTCTTG | |
| TUBA1 | tubulin, alpha 1 | CCGACTCAACG | 52 | CGTGGACTGAGA | 109 |
| | | TGAGAC | | TGCATT | |

TABLE 2

| | | | SEQ | | SEQ |
|---|---|---|---|---|---|
| Gene symbol | Gene name | Forward primer | ID | Reverse primer | ID |
| CDH5 | cadherin 5, type 2, | AAGAGAGATTG | 1 | TTCTTGCGACTCACGCT | 58 |
| | VE-cadherin | GATTTGGAACC | | | |
| | (vascular epithelium) | | | | |
| PAICS | phosphoribosylami | AATCCTGGTGT | 3 | GACCACTGTGGG | 60 |
| | noimidazole | CAAGGAAG | | TCATTATT | |
| | carboxylase, | | | | |
| | phosphoribosylami | | | | |
| | noimidazole | | | | |
| | succinocarboxamide | | | | |
| | synthetase | | | | |
| PAK1 | p21/Cdc42/Rac1- | GGACCGATTTT | 4 | GAAATCTCTGGC | 61 |
| | activated kinase 1 (STE20 | ACCGATCC | | CGCTC | |
| | homolog, yeast) | | | | |
| PECAM1 | platelet/endothelial cell | ACAGTCCAGAT | 5 | ACTGGGCATCAT | 62 |
| | adhesion molecule | AGTCGTATGT | | AAGAAATCC | |
| | (CD31 antigen) | | | | |
| TFAP2A | transcription factor AP- | GTCTCCGCCATC | 6 | ACTGAACAGAAG | 63 |
| | 2 alpha (activating | CCTAT | | ACATTCGT | |
| | enhancer binding | | | | |
| | protein 2 alpha) | | | | |
| ACVR1 | activin A receptor, | ACTGGTGTAAC | 7 | AACCTCCAAGTG | 64 |
| | type 1 | AGGAACAT | | GAAATTCT | |
| CDKN2C | cyclin-dependent kinase | TTTGGAAGGAC | 8 | TCGGTCTTTCAAA | 65 |
| | inhibitor 2C (p18, | TGCGCT | | TCGGGATTA | |
| | inhibits CDK4) | | | | |
| CIB1 | calcium and integrin | CACGTCATCTCC | 9 | CTGCTGTCACAG | 66 |
| | binding 1 (calmyrin) | CGTTC | | GACAAT | 66 |
| INSM1 | insulinoma-associated 1 | ATTGAACTTCCC | 10 | AAGGTAAAGCCA | 67 |
| | | ACACGA | | GACTCCA | 67 |
| ERP10 | low density lipoprotein | GGAACAGACTG | 11 | GGGAGCGTAGGG | 68 |
| | receptor-related protein | TCACCAT | | TTAAG | |
| | 10 | | | | |
| STMN1 | stathmin | TCAGAGTGTGTG | 12 | CAGTGTATTCTGC | 69 |
| | 1/oncoprotein 18 | G | | ACAATCAAC | |
| | | TCAGGC | | | |
| CAPG | capping protein (actin | GGGACAGCTTC | 13 | GTTCCAGGATGTT | 70 |
| | filament), gelsolin-like | AACACT | | GGACTTTC | |
| CHGA | chromogranin A | CCTGTGAACAG | 14 | GGAAAGTGTGTC | 71 |
| | (parathyroid secretory | CCCTATG | | GGAGAT | |
| | protein 1) | | | | |
| LGALS3 | lectin, galactoside- | TTCTGGGCACG | 15 | AGGCAACATCAT | 72 |
| | binding, soluble, 3 | GTGAAG | | TCCCTC | |
| | (galectin 3) | | | | |
| MAPRE3 | microtubule-associated | GGCCAAACTAG | 16 | GTCAACACCCAT | 73 |
| | protein, RP/EB family, | AGCACGAATA | | CTTCATTGAAA | |
| | member 3 | | | | |
| SFN | stratifin | TCAGCAAGAAG | 17 | CGTAGTGGAAGA | 74 |
| | | GAGATGCC | | CGGAAA | |
| SNAP91 | synaptosomal-associated | GTGCTCCCTCTC | 18 | CTGGTGTAGAATT | 75 |
| | protein, 91 kDa | CATTAAGTA | | AGGAGACGTA | |
| | homolog (mouse) | | | | |
| ABCC5 | ATP-binding cassette, | CAAGTTCAGGA | 19 | GGCATCAAGAGA | 76 |
| | sub-family C (CFTR/MRP), | GAACTCGAC | | GAGGC | |
| | member 5 | | | | |
| ALDH3B1 | aldehyde dehydrogenase | GGCTGTGGTTA | 20 | GATAAAGAGTTA | 77 |
| | 3 | TGCGATAG | | CAAGCTCCTCIG | |
| | family, member B1 | | | | |
| ANTXR1 | Anthrax toxin receptor 1 | ACCCGAGGAAC | 21 | TCTAGGCCTTGAC | 78 |
| | | AACCTTA | | GGAT | |
| CACNB1 | calcium channel, | CAGAGCGCCAG | 23 | GCACAGCAAATG | 80 |
| | voltage-dependent, beta | GCATTA | | CCACT | |
| | 1 subunit | | | | |
| CBX1 | chromobox homolog 1 | CCACTGGCTGA | 24 | CTTGTCTTTCCCT | 81 |
| | (HP1 bent homolog | GGTGTTA | | ACTGTCTTAC | |
| | Drosophila) | | | | |
| CYB5B | cytochrome b5 type B | TGGGCGAGTCT | 25 | CTTGTTCCAGCAG | 82 |
| | (outer mitochondrial | ACGATG | | AACCT | |
| | membrane) | | | | |
| DOK1 | docking protein 1, 62 | CTTTCTGCCCTG | 26 | CAGTCCTCTGCAC | 83 |
| | kDa (downstream of | GAGATG | | CGTTA | |
| | tyrosine kinase 1) | | | | |
| DSC3 | desmocollin 3 | GCGCCATTTGCT | 27 | CATCCAGATCCCT | 84 |
| | | AGAGATA | | CACAT | |
| FEN1 | flap structure-specific | AGAGAAGATGG | 28 | CCAAGACACAGC | 85 |
| | endonuclease 1 | GCAGAAAG | | CAGTAAT | |
| FOXH1 | forkhead box H1 | GCCCAGATCAT | 29 | TTTCCAGCCCTCG | 86 |
| | | CCGTCA | | TAGTC | |
| GJB5 | gap junction protein, | ACCACAAGGAC | 30 | GGGACACAGGGA | 87 |
| | beta 5 (connexin 31.1) | TTCGAC | | AGAAC | |
| HOXD1 | homeobox D1 | GCTCCGCTGCT | 31 | GTCTGCCACTCTG | 88 |
| | | ATCTTT | | CAAC | |
| HPN | Hepsin (transmembrane | AGCGGCCAGGT | 32 | GTCGGCTGACGC | 89 |
| | protease, serine 1) | GGATTA | | TTTGA | |
| HYAL2 | hyaluronoglucosam | ATGGGCTTTGG | 33 | GAACAAGTCAGT | 90 |
| | inidase 2 | GAGCATA | | CTAGGGAATAC | |
| ICA1 | islet cell autoantigen | GACCTGGATGC | 34 | TGCTTTCGATAAG | 91 |
| | 1, 69 kDa | CAAGCTA | | TCCAGACA | |
| ICAM5 | intercellular adhesion | CCGGCTCTTGG | 35 | CCTCTGAGGCTG | 92 |
| | molecule 5, | AAGTTG | | GAAACA | |
| | telencephalin | | | | |
| ITGA6 | integrin, alpha 6 | ACGCGGATCGA | 36 | ATCCACTGATCTT | 93 |
| | | GTTTGATAA | | CCTTGC | |
| LIPE | lipase, hormone-sensitive | CGCAAGTCCCA | 37 | CAGTGCTGCTTCA | 94 |
| | | GAAGAT | | GACACA | |
| ME3 | malic enzyme 3, | CGCGGATACGA | 38 | CCTTTCTTCAAGG | 95 |
| | NADP(+)-dependent, | TGTCAC | | GTAAAGGC | |
| | Mitochondrial | | | | |
| MGRN1 | mahogunin, ring finger | GAACTCGGCCT | 39 | TCGAATTTCTCTC | 96 |
| | 1 | ATCGCT | | CTCCCAT | |
| MYBPH | myosin binding protein | TCTGACCTCATC | 40 | CTGAGTCCACAC | 97 |
| | H | ATCGGCAA | | AGGTTT | |
| MYO7A | myosin VIIA | GAGGTGAAGCA | 41 | CCCATACTTGTTG | 98 |
| | | AACTACGGA | | ATGGCAATTA | |
| NFIL3 | nuclear factor, | ACTCTCCACAA | 42 | TCCTGCGTGTGTT | 99 |
| | interleukin 3 regulated | AGCTCG | | CTACT | |
| PIK3C2A | phosphoinositide-3-kinase | GGATTTCAGCT | 43 | AGTCATCATGTAC | 100 |
| | class 2, alpha | ACCAGTTACTT | | CCAGCA | |
| | polypeptide | | | | |
| PLEKHA6 | pleckstrin homology | TTCGTCCTGGTG | 44 | CCCAGGATACTCT | 101 |
| | domain containing, | GATCG | | CTTCCTT | |
| | family A member 6 | | | | |
| PSMD14 | proteasome (prosome, | AGTGATTGATG | 45 | CACTGGATCAAC | 102 |
| | macropain) 26S subunit, | TGTTTGCTATG | | TGCCTC | |
| | non-ATPase, 14 | | | | |
| SCD5 | stearoyk-CoA desaturase | CAAAGCCAAGC | 46 | CAGCTGTCACAC | 103 |
| | 5 | CACTCACTC | | CCAGAGC | |
| S1AH2 | seven in absentia | CTCGGCAGTCC | 47 | CGTATGGTGCAG | 104 |
| | homolog 2 | TGTTTC | | GGTCA | |
| | (Drosophila) | | | | |
| TCF2 | transcription factor 2, | ACACCTGGTAC | 48 | TCTGGACTGTCTG | 105 |
| | hepatic; LF-B3; variant | GTCACAA | | GTTGAAT | |
| | hepatic nuclear factor | | | | |
| TTF1 | thyroid transcription | ATGAGTCCAAA | 50 | CCATGCCCACTTT | 107 |
| | factor 1 | GCACACGA | | CTTGTA | |
| TRIM29 | tripartite motif-containing | TGAGATTGAGG | 51 | CATTGGTGGTGA | 108 |
| | 29 | ATGAAGCTGAG | | AGCTCTTG | |
| TUBA1 | tubulin, alpha 1 | CCGACTCAACG | 52 | CGTGGACTGAGA | 109 |
| | | TGAGAC | | TGCATT | |
| CFL1 | cofilin 1 (non-muscle) | GTGCCCTCTCCT | 53 | TTCATGTCGTTGA | 110 |
| | | TTTCG | | ACACCTTG | |
| EEF1A1 | eukaryotic translation | CGTTCTTTTTCG | 54 | CATTTTGGCTTTT | 111 |
| | elongation factor 1 | CAACGG | | AGGGGTAG | |
| | alpha 1 | | | | |
| RPL10 | ribosomal protein L10 | GGTGTGCCACT | 55 | GGCAGAAGCGAG | 112 |
| | | GAAGAT | | ACTTT | |
| RPL28 | ribosomal protein L28 | GTGTCGTGGTG | 56 | GCACATAGGAGG | 113 |
| | | GTCATT | | TGGCA | |
| RPL37A | ribosomal protein L37a | GCATGAAGACA | 57 | GCGGACTITACC | 114 |
| | | GTGGCT | | GTGAC | |

TABLE 3

| | | | SEQ | | SEQ |
|---|---|---|---|---|---|
| Gene symbol | Gene name | Forward primer | ID | Reverse primer | ID |
| CDH5 | cadherin 5, type 2, | AAGAGAGATTG | 1 | TTCTTGCGACTCACGCT | 58 |
| | VE-cadherin | GATTTGGAACC | | | |
| | (vascular epithelium) | | | | |
| CLEC3B | C-type lectin domain | CCAGAAGCCCA | 2 | GCTCCTCAAACAT | 59 |
| | family 3, member B | AGAAGATTGTA | | CTTTGTGTTCA | |
| PAICS | phosphoribosylami | AATCCTGGTGT | 3 | GACCACTGTGGG | 60 |
| | noimidazole | CAAGGAAG | | TCATTATT | |
| | carboxylase, | | | | |
| | phosphoribosylami | | | | |
| | noimidazole | | | | |
| | succinocarboxamide | | | | |
| | synthetase | | | | |
| PAK1 | p21/Cdc42/Rac1- | GGACCGATTTT | 4 | GAAATCTCTGGC | 61 |
| | activated kinase 1 (STE20 | ACCGATCC | | CGCTC | |
| | homolog, yeast) | | | | |
| TFAP2A | transcription factor AP- | GTCTCCGCCATC | 6 | ACTGAACAGAAG | 63 |
| | 2 alpha (activating | CCTAT | | ACATTCGT | |
| | enhancer binding | | | | |
| | protein 2 alpha) | | | | |
| ACVR1 | activin A receptor, | ACTGGTGTAAC | 7 | AACCTCCAAGTG | 64 |
| | type 1 | AGGAACAT | | GAAATTCT | |
| CDKN2C | cyclin-dependent kinase | TTTGGAAGGAC | 8 | TCGGTCTTTCAAA | 65 |
| | inhibitor 2C (p18, | TGCGCT | | TCGGGATTA | |
| | inhibits CDK4) | | | | |
| INSM1 | insulinoma-associated 1 | ATTGAACTTCCC | 10 | AAGGTAAAGCCA | 67 |
| | | ACACGA | | GACTCCA | 67 |
| ERP10 | low density lipoprotein | GGAACAGACTG | 11 | GGGAGCGTAGGG | 68 |
| | receptor-related protein | TCACCAT | | TTAAG | |
| | 10 | | | | |
| STMN1 | stathmin | TCAGAGTGTGTG | 12 | CAGTGTATTCTGC | 69 |
| | 1/oncoprotein 18 | G | | ACAATCAAC | |
| | | TCAGGC | | | |
| CAPG | capping protein (actin | GGGACAGCTTC | 13 | GTTCCAGGATGTT | 70 |
| | filament), gelsolin-like | AACACT | | GGACTTTC | |
| CHGA | chromogranin A | CCTGTGAACAG | 14 | GGAAAGTGTGTC | 71 |
| | (parathyroid secretory | CCCTATG | | GGAGAT | |
| | protein 1) | | | | |
| LGALS3 | lectin, galactoside- | TTCTGGGCACG | 15 | AGGCAACATCAT | 72 |
| | binding, soluble, 3 | GTGAAG | | TCCCTC | |
| | (galectin 3) | | | | |
| MAPRE3 | microtubule-associated | GGCCAAACTAG | 16 | GTCAACACCCAT | 73 |
| | protein, RP/EB family, | AGCACGAATA | | CTTCATTGAAA | |
| | member 3 | | | | |
| SFN | stratifin | TCAGCAAGAAG | 17 | CGTAGTGGAAGA | 74 |
| | | GAGATGCC | | CGGAAA | |
| SNAP91 | synaptosomal-associated | GTGCTCCCTCTC | 18 | CTGGTGTAGAATT | 75 |
| | protein, 91 kDa | CATTAAGTA | | AGGAGACGTA | |
| | homolog (mouse) | | | | |
| ABCC5 | ATP-binding cassette, | CAAGTTCAGGA | 19 | GGCATCAAGAGA | 76 |
| | sub-family C (CFTR/MRP), | GAACTCGAC | | GAGGC | |
| | member 5 | | | | |
| ALDH3B1 | aldehyde dehydrogenase | GGCTGTGGTTA | 20 | GATAAAGAGTTA | 77 |
| | 3 | TGCGATAG | | CAAGCTCCTCIG | |
| | family, member B1 | | | | |
| ANTXR1 | Anthrax toxin receptor 1 | ACCCGAGGAAC | 21 | TCTAGGCCTTGAC | 78 |
| | | AACCTTA | | GGAT | |
| CACNB1 | calcium channel, | CAGAGCGCCAG | 23 | GCACAGCAAATG | 80 |
| | voltage-dependent, beta | GCATTA | | CCACT | |
| | 1 subunit | | | | |
| CBX1 | chromobox homolog 1 | CCACTGGCTGA | 24 | CTTGTCTTTCCCT | 81 |
| | (HP1 bent homolog | GGTGTTA | | ACTGTCTTAC | |
| | Drosophila) | | | | |
| CYB5B | cytochrome b5 type B | TGGGCGAGTCT | 25 | CTTGTTCCAGCAG | 82 |
| | (outer mitochondrial | ACGATG | | AACCT | |
| | membrane) | | | | |
| DOK1 | docking protein 1, 62 | CTTTCTGCCCTG | 26 | CAGTCCTCTGCAC | 83 |
| | kDa (downstream of | GAGATG | | CGTTA | |
| | tyrosine kinase 1) | | | | |
| DSC3 | desmocollin 3 | GCGCCATTTGCT | 27 | CATCCAGATCCCT | 84 |
| | | AGAGATA | | CACAT | |
| FEN1 | flap structure-specific | AGAGAAGATGG | 28 | CCAAGACACAGC | 85 |
| | endonuclease 1 | GCAGAAAG | | CAGTAAT | |
| GJB5 | gap junction protein, | ACCACAAGGAC | 30 | GGGACACAGGGA | 87 |
| | beta 5 (connexin 31.1) | TTCGAC | | AGAAC | |
| HOXD1 | homeobox D1 | GCTCCGCTGCT | 31 | GTCTGCCACTCTG | 88 |
| | | ATCTTT | | CAAC | |
| HPN | Hepsin (transmembrane | AGCGGCCAGGT | 32 | GTCGGCTGACGC | 89 |
| | protease, serine 1) | GGATTA | | TTTGA | |
| HYAL2 | hyaluronoglucosam | ATGGGCTTTGG | 33 | GAACAAGTCAGT | 90 |
| | inidase 2 | GAGCATA | | CTAGGGAATAC | |
| ICA1 | islet cell autoantigen | GACCTGGATGC | 34 | TGCTTTCGATAAG | 91 |
| | 1, 69 kDa | CAAGCTA | | TCCAGACA | |
| ICAM5 | intercellular adhesion | CCGGCTCTTGG | 35 | CCTCTGAGGCTG | 92 |
| | molecule 5, | AAGTTG | | GAAACA | |
| | telencephalin | | | | |
| ITGA6 | integrin, alpha 6 | ACGCGGATCGA | 36 | ATCCACTGATCTT | 93 |
| | | GTTTGATAA | | CCTTGC | |
| ME3 | malic enzyme 3, | CGCGGATACGA | 38 | CCTTTCTTCAAGG | 95 |
| | NADP(+)-dependent, | TGTCAC | | GTAAAGGC | |
| | Mitochondrial | | | | |
| MGRN1 | mahogunin, ring finger | GAACTCGGCCT | 39 | TCGAATTTCTCTC | 96 |
| | 1 | ATCGCT | | CTCCCAT | |
| MYBPH | myosin binding protein | TCTGACCTCATC | 40 | CTGAGTCCACAC | 97 |
| | H | ATCGGCAA | | AGGTTT | |
| MYO7A | myosin VIIA | GAGGTGAAGCA | 41 | CCCATACTTGTTG | 98 |
| | | AACTACGGA | | ATGGCAATTA | |
| NFIL3 | nuclear factor, | ACTCTCCACAA | 42 | TCCTGCGTGTGTT | 99 |
| | interleukin 3 regulated | AGCTCG | | CTACT | |
| PIK3C2A | phosphoinositide-3-kinase | GGATTTCAGCT | 43 | AGTCATCATGTAC | 100 |
| | class 2, alpha | ACCAGTTACTT | | CCAGCA | |
| | polypeptide | | | | |
| PLEKHA6 | pleckstrin homology | TTCGTCCTGGTG | 44 | CCCAGGATACTCT | 101 |
| | domain containing, | GATCG | | CTTCCTT | |
| | family A member 6 | | | | |
| PSMD14 | proteasome (prosome, | AGTGATTGATG | 45 | CACTGGATCAAC | 102 |
| | macropain) 26S subunit, | TGTTTGCTATG | | TGCCTC | |
| | non-ATPase, 14 | | | | |
| SCD5 | stearoyk-CoA desaturase | CAAAGCCAAGC | 46 | CAGCTGTCACAC | 103 |
| | 5 | CACTCACTC | | CCAGAGC | |
| S1AH2 | seven in absentia | CTCGGCAGTCC | 47 | CGTATGGTGCAG | 104 |
| | homolog 2 | TGTTTC | | GGTCA | |
| | (Drosophila) | | | | |
| TCF2 | transcription factor 2, | ACACCTGGTAC | 48 | TCTGGACTGTCTG | 105 |
| | hepatic; LF-B3; variant | GTCACAA | | GTTGAAT | |
| | hepatic nuclear factor | | | | |
| TCP1 | t-complex 1 | ATGCCCAAGAG | 49 | CCTGTACACCAA | 106 |
| | | AATCGTAAA | | GCTTCAT | |
| TTF1 | thyroid transcription | ATGAGTCCAAA | 50 | CCATGCCCACTTT | 107 |
| | factor 1 | GCACACGA | | CTTGTA | |
| TRIM29 | tripartite motif-containing | TGAGATTGAGG | 51 | CATTGGTGGTGA | 108 |
| | 29 | ATGAAGCTGAG | | AGCTCTTG | |
| TUBA1 | tubulin, alpha 1 | CCGACTCAACG | 52 | CGTGGACTGAGA | 109 |
| | | TGAGAC | | TGCATT | |
| CFL1 | cofilin 1 (non-muscle) | GTGCCCTCTCCT | 53 | TTCATGTCGTTGA | 110 |
| | | TTTCG | | ACACCTTG | |
| EEF1A1 | eukaryotic translation | CGTTCTTTTTCG | 54 | CATTTTGGCTTTT | 111 |
| | elongation factor 1 | CAACGG | | AGGGGTAG | |
| | alpha 1 | | | | |
| RPL10 | ribosomal protein L10 | GGTGTGCCACT | 55 | GGCAGAAGCGAG | 112 |
| | | GAAGAT | | ACTTT | |
| RPL28 | ribosomal protein L28 | GTGTCGTGGTG | 56 | GCACATAGGAGG | 113 |
| | | GTCATT | | TGGCA | |
| RPL37A | ribosomal protein L37a | GCATGAAGACA | 57 | GCGGACTITACC | 114 |
| | | GTGGCT | | GTGAC | |

TABLE 4

| | | | SEQ | | SEQ |
|---|---|---|---|---|---|
| Gene symbol | Gene name | Forward primer | ID | Reverse primer | ID |
| ACVR1 | activin A receptor, | ACTGGTGTAAC | 7 | AACCTCCAAGTG | 64 |
| | type 1 | AGGAACAT | | GAAATTCT | |
| CDKN2C | cyclin-dependent kinase | TTTGGAAGGAC | 8 | TCGGTCTTTCAAA | 65 |
| | inhibitor 2C (p18, | TGCGCT | | TCGGGATTA | |
| | inhibits CDK4) | | | | |
| CIB1 | calcium and integrin | CACGTCATCTCC | 9 | CTGCTGTCACAG | 66 |
| | binding 1 (calmyrin) | CGTTC | | GACAAT | 66 |
| INSM1 | insulinoma-associated 1 | ATTGAACTTCCC | 10 | AAGGTAAAGCCA | 67 |
| | | ACACGA | | GACTCCA | 67 |
| ERP10 | low density lipoprotein | GGAACAGACTG | 11 | GGGAGCGTAGGG | 68 |
| | receptor-related protein | TCACCAT | | TTAAG | |
| | 10 | | | | |
| STMN1 | stathmin | TCAGAGTGTGTG | 12 | CAGTGTATTCTGC | 69 |
| | 1/oncoprotein 18 | G | | ACAATCAAC | |
| | | TCAGGC | | | |

TABLE 5

| | | | SEQ | | SEQ |
|---|---|---|---|---|---|
| Gene symbol | Gene name | Forward primer | ID | Reverse primer | ID |
| CAPG | capping protein (actin | GGGACAGCTTC | 13 | GTTCCAGGATGTT | 70 |
| | filament), gelsolin-like | AACACT | | GGACTTTC | |
| CHGA | chromogranin A | CCTGTGAACAG | 14 | GGAAAGTGTGTC | 71 |
| | (parathyroid secretory | CCCTATG | | GGAGAT | |
| | protein 1) | | | | |
| LGALS3 | lectin, galactoside- | TTCTGGGCACG | 15 | AGGCAACATCAT | 72 |
| | binding, soluble, 3 | GTGAAG | | TCCCTC | |
| | (galectin 3) | | | | |
| MAPRE3 | microtubule-associated | GGCCAAACTAG | 16 | GTCAACACCCAT | 73 |
| | protein, RP/EB family, | AGCACGAATA | | CTTCATTGAAA | |
| | member 3 | | | | |
| SFN | stratifin | TCAGCAAGAAG | 17 | CGTAGTGGAAGA | 74 |
| | | GAGATGCC | | CGGAAA | |
| SNAP91 | synaptosomal-associated | GTGCTCCCTCTC | 18 | CTGGTGTAGAATT | 75 |
| | protein, 91 kDa | CATTAAGTA | | AGGAGACGTA | |
| | homolog (mouse) | | | | |

TABLE 6

| | | | SEQ | | SEQ |
|---|---|---|---|---|---|
| Gene symbol | Gene name | Forward primer | ID | Reverse primer | ID |
| ABCC5 | ATP-binding cassette, | CAAGTTCAGGA | 19 | GGCATCAAGAGA | 76 |
| | sub-family C (CFTR/MRP), | GAACTCGAC | | GAGGC | |
| | member 5 | | | | |
| ALDH3B1 | aldehyde dehydrogenase | GGCTGTGGTTA | 20 | GATAAAGAGTTA | 77 |
| | 3 | TGCGATAG | | CAAGCTCCTCIG | |
| | family, member B1 | | | | |
| ANTXR1 | Anthrax toxin receptor 1 | ACCCGAGGAAC | 21 | TCTAGGCCTTGAC | 78 |
| | | AACCTTA | | GGAT | |
| BMP7 | Bone morphogenetic | CCCTCTCCATTCC | 22 | TTTGGGCAAACCTCGGTA | 79 |
| | protein 7 (osteogenic | CTACA | | A | |
| | protein 1) | | | | |
| CACNB1 | calcium channel, | CAGAGCGCCAG | 23 | GCACAGCAAATG | 80 |
| | voltage-dependent, beta | GCATTA | | CCACT | |
| | 1 subunit | | | | |
| CBX1 | chromobox homolog 1 | CCACTGGCTGA | 24 | CTTGTCTTTCCCT | 81 |
| | (HP1 bent homolog | GGTGTTA | | ACTGTCTTAC | |
| | Drosophila) | | | | |
| CYB5B | cytochrome b5 type B | TGGGCGAGTCT | 25 | CTTGTTCCAGCAG | 82 |
| | (outer mitochondrial | ACGATG | | AACCT | |
| | membrane) | | | | |
| DOK1 | docking protein 1, 62 | CTTTCTGCCCTG | 26 | CAGTCCTCTGCAC | 83 |
| | kDa (downstream of | GAGATG | | CGTTA | |
| | tyrosine kinase 1) | | | | |
| DSC3 | desmocollin 3 | GCGCCATTTGCT | 27 | CATCCAGATCCCT | 84 |
| | | AGAGATA | | CACAT | |
| FEN1 | flap structure-specific | AGAGAAGATGG | 28 | CCAAGACACAGC | 85 |
| | endonuclease 1 | GCAGAAAG | | CAGTAAT | |
| FOXH1 | forkhead box H1 | GCCCAGATCAT | 29 | TTTCCAGCCCTCG | 86 |
| | | CCGTCA | | TAGTC | |
| GJB5 | gap junction protein, | ACCACAAGGAC | 30 | GGGACACAGGGA | 87 |
| | beta 5 (connexin 31.1) | TTCGAC | | AGAAC | |
| HOXD1 | homeobox D1 | GCTCCGCTGCT | 31 | GTCTGCCACTCTG | 88 |
| | | ATCTTT | | CAAC | |
| HPN | Hepsin (transmembrane | AGCGGCCAGGT | 32 | GTCGGCTGACGC | 89 |
| | protease, serine 1) | GGATTA | | TTTGA | |
| HYAL2 | hyaluronoglucosam | ATGGGCTTTGG | 33 | GAACAAGTCAGT | 90 |
| | inidase 2 | GAGCATA | | CTAGGGAATAC | |
| ICA1 | islet cell autoantigen | GACCTGGATGC | 34 | TGCTTTCGATAAG | 91 |
| | 1, 69 kDa | CAAGCTA | | TCCAGACA | |
| ICAM5 | intercellular adhesion | CCGGCTCTTGG | 35 | CCTCTGAGGCTG | 92 |
| | molecule 5, | AAGTTG | | GAAACA | |
| | telencephalin | | | | |
| ITGA6 | integrin, alpha 6 | ACGCGGATCGA | 36 | ATCCACTGATCTT | 93 |
| | | GTTTGATAA | | CCTTGC | |
| LIPE | lipase, hormone-sensitive | CGCAAGTCCCA | 37 | CAGTGCTGCTTCA | 94 |
| | | GAAGAT | | GACACA | |
| ME3 | malic enzyme 3, | CGCGGATACGA | 38 | CCTTTCTTCAAGG | 95 |
| | NADP(+)-dependent, | TGTCAC | | GTAAAGGC | |
| | Mitochondrial | | | | |
| MGRN1 | mahogunin, ring finger | GAACTCGGCCT | 39 | TCGAATTTCTCTC | 96 |
| | 1 | ATCGCT | | CTCCCAT | |
| MYBPH | myosin binding protein | TCTGACCTCATC | 40 | CTGAGTCCACAC | 97 |
| | H | ATCGGCAA | | AGGTTT | |
| MYO7A | myosin VIIA | GAGGTGAAGCA | 41 | CCCATACTTGTTG | 98 |
| | | AACTACGGA | | ATGGCAATTA | |
| NFIL3 | nuclear factor, | ACTCTCCACAA | 42 | TCCTGCGTGTGTT | 99 |
| | interleukin 3 regulated | AGCTCG | | CTACT | |
| PIK3C2A | phosphoinositide-3-kinase | GGATTTCAGCT | 43 | AGTCATCATGTAC | 100 |
| | class 2, alpha | ACCAGTTACTT | | CCAGCA | |
| | polypeptide | | | | |
| PLEKHA6 | pleckstrin homology | TTCGTCCTGGTG | 44 | CCCAGGATACTCT | 101 |
| | domain containing, | GATCG | | CTTCCTT | |
| | family A member 6 | | | | |
| PSMD14 | proteasome (prosome, | AGTGATTGATG | 45 | CACTGGATCAAC | 102 |
| | macropain) 26S subunit, | TGTTTGCTATG | | TGCCTC | |
| | non-ATPase, 14 | | | | |
| SCD5 | stearoyk-CoA desaturase | CAAAGCCAAGC | 46 | CAGCTGTCACAC | 103 |
| | 5 | CACTCACTC | | CCAGAGC | |
| S1AH2 | seven in absentia | CTCGGCAGTCC | 47 | CGTATGGTGCAG | 104 |
| | homolog 2 | TGTTTC | | GGTCA | |
| | (Drosophila) | | | | |
| TCF2 | transcription factor 2, | ACACCTGGTAC | 48 | TCTGGACTGTCTG | 105 |
| | hepatic; LF-B3; variant | GTCACAA | | GTTGAAT | |
| | hepatic nuclear factor | | | | |
| TCP1 | t-complex 1 | ATGCCCAAGAG | 49 | CCTGTACACCAA | 106 |
| | | AATCGTAAA | | GCTTCAT | |
| TTF1 | thyroid transcription | ATGAGTCCAAA | 50 | CCATGCCCACTTT | 107 |
| | factor 1 | GCACACGA | | CTTGTA | |
| TRIM29 | tripartite motif-containing | TGAGATTGAGG | 51 | CATTGGTGGTGA | 108 |
| | 29 | ATGAAGCTGAG | | AGCTCTTG | |
| TUBA1 | tubulin, alpha 1 | CCGACTCAACG | 52 | CGTGGACTGAGA | 109 |
| | | TGAGAC | | TGCATT | |

Isolated mRNA can be used in hybridization or amplification assays that include, but are not limited to, Southern or Northern analyses, PCR analyses and probe arrays, NanoString Assays. One method for the detection of mRNA levels involves contacting the isolated mRNA or synthesized cDNA with a nucleic acid molecule (probe) that can hybridize to the mRNA encoded by the gene being detected. The nucleic acid probe can be, for example, a cDNA, or a portion thereof, such as an oligonucleotide of at least 7, 15, 30, 50, 100, 250, or 500 nucleotides in length and sufficient to specifically hybridize under stringent conditions to the non-natural cDNA or mRNA biomarker of the present invention.

As explained above, in one embodiment, once the mRNA is obtained from a sample, it is converted to complementary DNA (cDNA) in a hybridization reaction. Conversion of the mRNA to cDNA can be performed with oligonucleotides or primers comprising sequence that is complementary to a portion of a specific mRNA. Conversion of the mRNA to cDNA can be performed with oligonucleotides or primers comprising random sequence. Conversion of the mRNA to cDNA can be performed with oligonucleotides or primers comprising sequence that is complementary to the poly(A) tail of an mRNA. cDNA does not exist in vivo and therefore is a non-natural molecule. In a further embodiment, the cDNA is then amplified, for example, by the polymerase chain reaction (PCR) or other amplification method known to those of ordinary skill in the art. PCR can be performed with the forward and/or reverse primers provided in Table 1A, Table 1B, Table 1C, Table 2, Table 3, Table 4, Table 5, or Table 6. The product of this amplification reaction, i.e., amplified cDNA is necessarily a non-natural product. As mentioned above, cDNA is a non-natural molecule. Second, in the case of PCR, the amplification process serves to create hundreds of millions of cDNA copies for every individual cDNA molecule of starting material. The number of copies generated is far removed from the number of copies of mRNA that are present in vivo.

In one embodiment, cDNA is amplified with primers that introduce an additional DNA sequence (adapter sequence) onto the fragments (with the use of adapter-specific primers). The adaptor sequence can be a tail, wherein the tail sequence is not complementary to the cDNA. For example, the forward and/or reverse primers provided in Table 1A, Table 1B, Table 1C, Table 2, Table 3, Table 4, Table 5, or Table 6 can comprise tail sequence. Amplification therefore serves to create non-natural double stranded molecules from the non-natural single stranded cDNA, by introducing barcode, adapter and/or reporter sequences onto the already non-natural cDNA. In one embodiment, during amplification with the adapter-specific primers, a detectable label, e.g., a fluorophore, is added to single strand cDNA molecules. Amplification therefore also serves to create DNA complexes that do not occur in nature, at least because (i) cDNA does not exist in vivo, (i) adapter sequences are added to the ends of cDNA molecules to make DNA sequences that do not exist in vivo, (ii) the error rate associated with amplification further creates DNA sequences that do not exist in vivo, (iii) the disparate structure of the cDNA molecules as compared to what exists in nature and (iv) the chemical addition of a detectable label to the cDNA molecules.

In one embodiment, the synthesized cDNA (for example, amplified cDNA) is immobilized on a solid surface via hybridization with a probe, e.g., via a microarray. In another embodiment, cDNA products are detected via real-time polymerase chain reaction (PCR) via the introduction of fluorescent probes that hybridize with the cDNA products. For example, in one embodiment, biomarker detection is assessed by quantitative fluorogenic RT-PCR (e.g., with TaqMan® probes). For PCR analysis, well known methods are available in the art for the determination of primer sequences for use in the analysis.

Biomarkers provided herein in one embodiment, are detected via a hybridization reaction that employs a capture probe and/or a reporter probe. For example, the hybridization probe is a probe derivatized to a solid surface such as a bead, glass or silicon substrate. In another embodiment, the capture probe is present in solution and mixed with the patient's sample, followed by attachment of the hybridization product to a surface, e.g., via a biotin-avidin interaction (e.g., where biotin is a part of the capture probe and avidin is on the surface). The hybridization assay, in one embodiment, employs both a capture probe and a reporter probe. The reporter probe can hybridize to either the capture probe or the biomarker nucleic acid. Reporter probes e.g., are then counted and detected to determine the level of biomarker(s) in the sample. The capture and/or reporter probe, in one embodiment contain a detectable label, and/or a group that allows functionalization to a surface.

For example, the nCounter gene analysis system (see, e.g., Geiss et al. (2008) Nat. Biotechnol. 26, pp. 317-325, incorporated by reference in its entirety for all purposes, is amenable for use with the methods provided herein.

Hybridization assays described in U.S. Pat. Nos. 7,473,767 and 8,492,094, the disclosures of which are incorporated by reference in their entireties for all purposes, are amenable for use with the methods provided herein, i.e., to detect the biomarkers and biomarker combinations described herein.

Biomarker levels may be monitored using a membrane blot (such as used in hybridization analysis such as Northern, Southern, dot, and the like), or microwells, sample tubes, gels, beads, or fibers (or any solid support comprising bound nucleic acids). See, for example, U.S. Pat. Nos. 5,770,722, 5,874,219, 5,744,305, 5,677,195 and 5,445,934, each incorporated by reference in their entireties.

In one embodiment, microarrays are used to detect biomarker levels. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316, each incorporated by reference in their entireties. High-density oligonucleotide arrays are particularly useful for determining the gene expression profile for a large number of RNAs in a sample.

Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992, each incorporated by reference in their entireties. Arrays can be packaged in such a manner as to allow for diagnostics or other manipulation of an all-inclusive device. See, for example, U.S. Pat. Nos. 5,856,174 and 5,922,591, each incorporated by reference in their entireties.

Serial analysis of gene expression (SAGE) in one embodiment is employed in the methods described herein. SAGE is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. See, Velculescu et al. Science 270:484-87, 1995; Cell 88:243-51, 1997, incorporated by reference in its entirety.

An additional method of biomarker level analysis at the nucleic acid level is the use of a sequencing method, for example, RNAseq, next generation sequencing, and massively parallel signature sequencing (MPSS), as described by Brenner et al. (Nat. Biotech. 18:630-34, 2000, incorporated by reference in its entirety). This is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3.0×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Another method if biomarker level analysis at the nucleic acid level is the use of an amplification method such as, for example, RT-PCR or quantitative RT-PCR (qRT-PCR). Methods for determining the level of biomarker mRNA in a sample may involve the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Numerous different PCR or qRT-PCR protocols are known in the art and can be directly applied or adapted for use using the presently described compositions for the detection and/or quantification of expression of discriminative genes in a sample. See, for example, Fan et al. (2004) Genome Res. 14:878-885, herein incorporated by reference. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR.

Quantitative RT-PCR (qRT-PCR) (also referred as real-time RT-PCR) is preferred under some circumstances because it provides not only a quantitative measurement, but also reduced time and contamination. As used herein, “quantitative PCR (or “real time qRT-PCR”) refers to the direct monitoring of the progress of a PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. A DNA binding dye (e.g., SYBR green) or a labeled probe can be used to detect the extension product generated by PCR amplification. Any probe format utilizing a labeled probe comprising the sequences of the invention may be used.

Immunohistochemistry methods are also suitable for detecting the levels of the biomarkers of the present invention. Samples can be frozen for later preparation or immediately placed in a fixative solution. Tissue samples can be fixed by treatment with a reagent, such as formalin, gluteraldehyde, methanol, or the like and embedded in paraffin. Methods for preparing slides for immunohistochemical analysis from formalin-fixed, paraffin-embedded tissue samples are well known in the art.

In one embodiment, the levels of the biomarkers of Table 1A, Table 1B, Table 1C, Table 2, Table 3, Table 4, Table 5 or Table 6 (or subsets thereof, for example 5 to 20, 5 to 30, 5 to 40 biomarkers), are normalized against the expression levels of all RNA transcripts or their non-natural cDNA expression products, or protein products in the sample, or of a reference set of RNA transcripts or a reference set of their non-natural cDNA expression products, or a reference set of their protein products in the sample.

As provided throughout, the methods set forth herein provide a method for determining the lung cancer subtype of a patient. Once the biomarker levels are determined, for example by measuring non-natural cDNA biomarker levels or non-natural mRNA-cDNA biomarker complexes, the biomarker levels are compared to reference values or a reference sample, for example with the use of statistical methods or direct comparison of detected levels, to make a determination of the lung cancer molecular subtype. Based on the comparison, the patient's lung cancer sample is classified, e.g., as neuroendocrine, squamous cell carcinoma, adenocarcinoma. In another embodiment, based on the comparison, the patient's lung cancer sample is classified as squamous cell carcinoma, adenocarcinoma or small cell carcinoma. In yet another embodiment, based on the comparison, the patient's lung cancer sample is classified as squamoid (proximal inflammatory), bronchoid (terminal respiratory unit) or magnoid (proximal proliferative).

In one embodiment, expression level values of the at least five classifier biomarkers of Table 1A, Table 1B, Table 1C, Table 2, Table 3, Table 4, Table 5 or Table 6 are compared to reference expression level value(s) from at least one sample training set, wherein the at least one sample training set comprises expression level values from a reference sample(s). In a further embodiment, the at least one sample training set comprises expression level values of the at least five classifier biomarkers of Table 1A, Table 1B, Table 1C, Table 2, Table 3, Table 4, Table 5 or Table 6 from an adenocarcinoma sample, a squamous cell carcinoma sample, a neuroendocrine sample, a small cell lung carcinoma sample, a proximal inflammatory (squamoid), proximal proliferative (magnoid), a terminal respiratory unit (bronchioid) sample, or a combination thereof.

In a separate embodiment, hybridization values of the at least five classifier biomarkers of Table 1A, Table 1B, Table 1C, Table 2, Table 3, Table 4, Table 5 or Table 6 are compared to reference hybridization value(s) from at least one sample training set, wherein the at least one sample training set comprises hybridization values from a reference sample(s). In a further embodiment, the at least one sample training set comprises hybridization values of the at least five classifier biomarkers of Table 1A, Table 1B, Table 1C, Table 2, Table 3, Table 4, Table 5 or Table 6 from an adenocarcinoma sample, a squamous cell carcinoma sample, a neuroendocrine sample, a small cell lung carcinoma sample, a proximal inflammatory (squamoid), proximal proliferative (magnoid), a terminal respiratory unit (bronchioid) sample, or a combination thereof. In another embodiment, the at least one sample training set comprises hybridization values of the at least five classifier biomarkers of Table 1A, Table 1B, Table 1C, Table 2, Table 3, Table 4, Table 5, Table 6 from the reference samples provided in Table A below.

TABLE A
Various sample training set embodiments of the invention

| At least one sample | Origin of reference sample | Lung cancer subtyping |
|---|---|---|
| training set | hybridization values | method |
| Embodiment 1 | Adenocarcinoma reference sample | Assessing whether patient |
| | and/or squamous cell carcinoma | sample is adenocarcinoma or |
| | reference sample | squamous cell carcinoma |
| Embodiment 2 | Adenocarcinoma reference | Assessing whether patient |
| | sample, squamous cell carcinoma | sample is adenocarcinoma, |
| | reference sample and/or | squamous cell carcinoma or |
| | neuroendocrine reference sample | neuroendocrine sample |
| Embodiment 3 | Adenocarcinoma reference | Assessing whether patient |
| | sample, squamous cell carcinoma | sample is adenocarcinoma, |
| | reference sample and/or small | squamous cell carcinoma or |
| | cell carcinoma reference sample | small cell carcinoma sample |
| Embodiment 4 | proximal inflammatory | Assessing whether patient |
| | (squamoid) reference sample, | sample is proximal inflammatory |
| | proximal proliferative | (squamoid), proximal |
| | (magnoid), and/or terminal | proliferative (magnoid), or |
| | respiratory unit | terminal respiratory unit |
| | (bronchioid) sample | (bronchioid) |

Methods for comparing detected levels of biomarkers to reference values and/or reference samples are provided herein. Based on this comparison, in one embodiment a correlation between the biomarker levels obtained from the subject's sample and the reference values is obtained. An assessment of the lung cancer subtype is then made.

Various statistical methods can be used to aid in the comparison of the biomarker levels obtained from the patient and reference biomarker levels, for example, from at least one sample training set.

In one embodiment, a supervised pattern recognition method is employed. Examples of supervised pattern recognition methods can include, but are not limited to, the nearest centroid methods (Dabney (2005) Bioinformatics 21(22):4148-4154 and Tibshirani et al. (2002) Proc. Natl. Acad. Sci. USA 99(10):6576-6572); soft independent modeling of class analysis (SIMCA) (see, for example, Wold, 1976); partial least squares analysis (PLS) (see, for example, Wold, 1966; Joreskog, 1982; Frank, 1984; Bro, R., 1997); linear discriminant analysis (LDA) (see, for example, Nillson, 1965); K-nearest neighbour analysis (KNN) (sec, for example, Brown et al., 1996); artificial neural networks (ANN) (see, for example, Wasserman, 1989; Anker et al., 1992; Hare, 1994); probabilistic neural networks (PNNs) (see, for example, Parzen, 1962; Bishop, 1995; Speckt, 1990; Broomhead et al., 1988; Patterson, 1996); rule induction (RI) (see, for example, Quinlan, 1986); and, Bayesian methods (see, for example, Bretthorst, 1990a, 1990b, 1988). In one embodiment, the classifier for identifying tumor subtypes based on gene expression data is the centroid based method described in Mullins et al. (2007) Clin Chem. 53(7):1273-9, each of which is herein incorporated by reference in its entirety.

In other embodiments, an unsupervised training approach is employed, and therefore, no training set is used.

Referring to sample training sets for supervised learning approaches again, in some embodiments, a sample training set(s) can include expression data of all of the classifier biomarkers (e.g., all the classifier biomarkers of any of Table 1A, Table 1B, Table 1C, Table 2, Table 3, Table 4, Table 5, Table 6) from an adenocarcinoma sample. In some embodiments, a sample training set(s) can include expression data of all of the classifier biomarkers (e.g., all the classifier biomarkers of any of Table 1A, Table 1B, Table 1C, Table 2, Table 3, Table 4, Table 5, Table 6) from a squamous cell carcinoma sample, an adenocarcinoma sample and/or a neuroendocrine sample. In some embodiments, the sample training set(s) are normalized to remove sample-to-sample variation.

In some embodiments, comparing can include applying a statistical algorithm, such as, for example, any suitable multivariate statistical analysis model, which can be parametric or non-parametric. In some embodiments, applying the statistical algorithm can include determining a correlation between the expression data obtained from the human lung tissue sample and the expression data from the adenocarcinoma and squamous cell carcinoma training set(s). In some embodiments, cross-validation is performed, such as (for example), leave-one-out cross-validation (LOOCV). In some embodiments, integrative correlation is performed. In some embodiments, a Spearman correlation is performed. In some embodiments, a centroid based method is employed for the statistical algorithm as described in Mullins et al. (2007) Clin Chem. 53(7):1273-9, and based on gene expression data, which is herein incorporated by reference in its entirety.

Results of the gene expression performed on a sample from a subject (test sample) may be compared to a biological sample(s) or data derived from a biological sample(s) that is known or suspected to be normal (“reference sample” or “normal sample”, e.g., non-adenocarcinoma sample). In some embodiments, a reference sample or reference gene expression data is obtained or derived from an individual known to have a particular molecular subtype of adenocarcinoma, i.e., squamoid (proximal inflammatory), bronchoid (terminal respiratory unit) or magnoid (proximal proliferative). In another embodiment, a reference sample or reference biomarker level data is obtained or derived from an individual known to have a lung cancer subtype, e.g., adenocarcinoma, squamous cell carcinoma, neuroendocrine or small cell carcinoma.

The reference sample may be assayed at the same time, or at a different time from the test sample. Alternatively, the biomarker level information from a reference sample may be stored in a database or other means for access at a later date.

The biomarker level results of an assay on the test sample may be compared to the results of the same assay on a reference sample. In some cases, the results of the assay on the reference sample are from a database, or a reference value(s). In some cases, the results of the assay on the reference sample are a known or generally accepted value or range of values by those skilled in the art. In some cases the comparison is qualitative. In other cases the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, expression levels of the genes described herein, mRNA copy numbers.

In one embodiment, an odds ratio (OR) is calculated for each biomarker level panel measurement. Here, the OR is a measure of association between the measured biomarker values for the patient and an outcome, e.g., lung cancer subtype. For example, see, J. Can. Acad. Child Adolesc. Psychiatry 2010; 19(3): 227-229, which is incorporated by reference in its entirety for all purposes.

In one embodiment, a specified statistical confidence level may be determined in order to provide a confidence level regarding the lung cancer subtype. For example, it may be determined that a confidence level of greater than 90% may be a useful predictor of the lung cancer subtype. In other embodiments, more or less stringent confidence levels may be chosen. For example, a confidence level of about or at least about 50%, 60%, 70%, 75%, 80%, 85%, 90%, 95%, 97.5%, 99%, 99.5%, or 99.9% may be chosen. The confidence level provided may in some cases be related to the quality of the sample, the quality of the data, the quality of the analysis, the specific methods used, and/or the number of gene expression values (i.e., the number of genes) analyzed. The specified confidence level for providing the likelihood of response may be chosen on the basis of the expected number of false positives or false negatives. Methods for choosing parameters for achieving a specified confidence level or for identifying markers with diagnostic power include but are not limited to Receiver Operating Characteristic (ROC) curve analysis, binormal ROC, principal component analysis, odds ratio analysis, partial least squares analysis, singular value decomposition, least absolute shrinkage and selection operator analysis, least angle regression, and the threshold gradient directed regularization method.

Determining the lung cancer subtype in some cases can be improved through the application of algorithms designed to normalize and or improve the reliability of the gene expression data. In some embodiments of the present invention, the data analysis utilizes a computer or other device, machine or apparatus for application of the various algorithms described herein due to the large number of individual data points that are processed. A “machine learning algorithm” refers to a computational-based prediction methodology, also known to persons skilled in the art as a “classifier,” employed for characterizing a gene expression profile or profiles, e.g., to determine the lung cancer subtype. The biomarker levels, determined by, e.g., microarray-based hybridization assays, sequencing assays, NanoString assays, etc., are in one embodiment subjected to the algorithm in order to classify the profile. Supervised learning generally involves “training” a classifier to recognize the distinctions among classes (e.g., adenocarcinoma positive, adenocarcinoma negative, squamous positive, squamous negative, neuroendocrine positive, neuroendocrine negative, small cell positive, small cell negative, squamoid (proximal inflammatory) positive, bronchoid (terminal respiratory unit) positive or magnoid (proximal proliferative) positive, and then “testing” the accuracy of the classifier on an independent test set. For new, unknown samples the classifier can be used to predict, for example, the class (e.g., adenocarcinoma vs. squamous cell carcinoma vs. neuroendocrine) in which the samples belong.

In some embodiments, a robust multi-array average (RMA) method may be used to normalize raw data. The RMA method begins by computing background-corrected intensities for each matched cell on a number of microarrays. In one embodiment, the background corrected values are restricted to positive values as described by Irizarry et al. (2003). Biostatistics April 4 (2): 249-64, incorporated by reference in its entirety for all purposes. After background correction, the base-2 logarithm of each background corrected matched-cell intensity is then obtained. The background corrected, log-transformed, matched intensity on each microarray is then normalized using the quantile normalization method in which for each input array and each probe value, the array percentile probe value is replaced with the average of all array percentile points, this method is more completely described by Bolstad et al. Bioinformatics 2003, incorporated by reference in its entirety. Following quantile normalization, the normalized data may then be fit to a linear model to obtain an intensity measure for each probe on each microarray. Tukey's median polish algorithm (Tukey, J. W., Exploratory Data Analysis. 1977, incorporated by reference in its entirety for all purposes) may then be used to determine the log-scale intensity level for the normalized probe set data.

Various other software programs may be implemented. In certain methods, feature selection and model estimation may be performed by logistic regression with lasso penalty using glmnet (Friedman et al. (2010). Journal of statistical software 33(1): 1-22, incorporated by reference in its entirety). Raw reads may be aligned using TopHat (Trapnell et al. (2009). Bioinformatics 25(9): 1105-11, incorporated by reference in its entirety). In methods, top features (N ranging from 10 to 200) are used to train a linear support vector machine (SVM) (Suykens J A K, Vandewalle J. Least Squares Support Vector Machine Classifiers. Neural Processing Letters 1999; 9(3): 293-300, incorporated by reference in its entirety) using the e1071 library (Meyer D. Support vector machines: the interface to libsvm in package e1071. 2014, incorporated by reference in its entirety). Confidence intervals, in one embodiment, are computed using the pROC package (Robin X, Turck N, Hainard A, et al. pROC: an open-source package for R and S+ to analyze and compare ROC curves. BMC bioinformatics 2011; 12: 77, incorporated by reference in its entirety).

In addition, data may be filtered to remove data that may be considered suspect. In one embodiment, data derived from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may in one embodiment be considered unreliable due to their aberrant hybridization propensity or secondary structure issues.

In some embodiments of the present invention, data from probe-sets may be excluded from analysis if they are not identified at a detectable level (above background).

In some embodiments of the present disclosure, probe-sets that exhibit no, or low variance may be excluded from further analysis. Low-variance probe-sets are excluded from the analysis via a Chi-Square test. In one embodiment, a probe-set is considered to be low-variance if its transformed variance is to the left of the 99 percent confidence interval of the Chi-Squared distribution with (N-l) degrees of freedom. (N-l)*Probe-set Variance/(Gene Probe-set Variance). about.Chi-Sq(N-l) where N is the number of input CEL files, (N-l) is the degrees of freedom for the Chi-Squared distribution, and the “probe-set variance for the gene” is the average of probe-set variances across the gene. In some embodiments of the present invention, probe-sets for a given mRNA or group of mRNAs may be excluded from further analysis if they contain less than a minimum number of probes that pass through the previously described filter steps for GC content, reliability, variance and the like. For example in some embodiments, probe-sets for a given gene or transcript cluster may be excluded from further analysis if they contain less than about 1, 2, 3, 4, 5, 6, 7, 8, 9, 10, 11, 12, 13, 14, 15, or less than about 20 probes.

Methods of biomarker level data analysis in one embodiment, further include the use of a feature selection algorithm as provided herein. In some embodiments of the present invention, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420, incorporated by reference in its entirety for all purposes).

Methods of biomarker level data analysis, in one embodiment, include the use of a pre-classifier algorithm. For example, an algorithm may use a specific molecular fingerprint to pre-classify the samples according to their composition and then apply a correction/normalization factor. This data/information may then be fed in to a final classification algorithm which would incorporate that information to aid in the final diagnosis.

Methods of biomarker level data analysis, in one embodiment, further include the use of a classifier algorithm as provided herein. In one embodiment of the present invention, a diagonal linear discriminant analysis, k-nearest neighbor algorithm, support vector machine (SVM) algorithm, linear support vector machine, random forest algorithm, or a probabilistic model-based method or a combination thereof is provided for classification of microarray data. In some embodiments, identified markers that distinguish samples (e.g., of varying biomarker level profiles, of varying lung cancer subtypes, and/or varying molecular subtypes of adenocarcinoma (e.g., squamoid, bronchoid, magnoid)) are selected based on statistical significance of the difference in biomarker levels between classes of interest. In some cases, the statistical significance is adjusted by applying a Benjamin Hochberg or another correction for false discovery rate (FDR).

In some cases, the classifier algorithm may be supplemented with a meta-analysis approach such as that described by Fishel and Kaufman et al. 2007 Bioinformatics 23(13): 1599-606, incorporated by reference in its entirety for all purposes. In some cases, the classifier algorithm may be supplemented with a meta-analysis approach such as a repeatability analysis.

Methods for deriving and applying posterior probabilities to the analysis of biomarker level data are known in the art and have been described for example in Smyth, G. K. 2004 Stat. Appi. Genet. Mol. Biol. 3: Article 3, incorporated by reference in its entirety for all purposes. In some cases, the posterior probabilities may be used in the methods of the present invention to rank the markers provided by the classifier algorithm.

A statistical evaluation of the results of the biomarker level profiling may provide a quantitative value or values indicative of one or more of the following: the lung cancer subtype (adenocarcinoma, squamous cell carcinoma, neuroendocrine); molecular subtype of adenocarcinoma (squamoid, bronchoid or magnoid); the likelihood of the success of a particular therapeutic intervention, e.g., angiogenesis inhibitor therapy or chemotherapy. In one embodiment, the data is presented directly to the physician in its most useful form to guide patient care, or is used to define patient populations in clinical trials or a patient population for a given medication. The results of the molecular profiling can be statistically evaluated using a number of methods known to the art including, but not limited to: the students T test, the two sided T test, Pearson rank sum analysis, hidden Markov model analysis, analysis of q-q plots, principal component analysis, one way ANOVA, two way ANOVA, LIMMA and the like.

In some cases, accuracy may be determined by tracking the subject over time to determine the accuracy of the original diagnosis. In other cases, accuracy may be established in a deterministic manner or using statistical methods. For example, receiver operator characteristic (ROC) analysis may be used to determine the optimal assay parameters to achieve a specific level of accuracy, specificity, positive predictive value, negative predictive value, and/or false discovery rate.

In some cases the results of the biomarker level profiling assays, are entered into a database for access by representatives or agents of a molecular profiling business, the individual, a medical provider, or insurance provider. In some cases, assay results include sample classification, identification, or diagnosis by a representative, agent or consultant of the business, such as a medical professional. In other cases, a computer or algorithmic analysis of the data is provided automatically. In some cases the molecular profiling business may bill the individual, insurance provider, medical provider, researcher, or government entity for one or more of the following: molecular profiling assays performed, consulting services, data analysis, reporting of results, or database access.

In some embodiments of the present invention, the results of the biomarker level profiling assays are presented as a report on a computer screen or as a paper record. In some embodiments, the report may include, but is not limited to, such information as one or more of the following: the levels of biomarkers (e.g., as reported by copy number or fluorescence intensity, etc.) as compared to the reference sample or reference value(s); the likelihood the subject will respond to a particular therapy, based on the biomarker level values and the lung cancer subtype and proposed therapies.

In one embodiment, the results of the gene expression profiling may be classified into one or more of the following: adenocarcinoma positive, adenocarcinoma negative, squamous cell carcinoma positive, squamous cell carcinoma negative, neuroendocrine positive, neuroendocrine negative, small cell carcinoma positive, small cell carcinoma negative, squamoid (proximal inflammatory) positive, bronchoid (terminal respiratory unit) positive, magnoid (proximal proliferative) positive, squamoid (proximal inflammatory) negative, bronchoid (terminal respiratory unit) negative, magnoid (proximal proliferative) negative; likely to respond to angiogenesis inhibitor or chemotherapy; unlikely to respond to angiogenesis inhibitor or chemotherapy; or a combination thereof.

In some embodiments of the present invention, results are classified using a trained algorithm. Trained algorithms of the present invention include algorithms that have been developed using a reference set of known gene expression values and/or normal samples, for example, samples from individuals diagnosed with a particular molecular subtype of adenocarcinoma. In some cases a reference set of known gene expression values are obtained from individuals who have been diagnosed with a particular molecular subtype of adenocarcinoma, and are also known to respond (or not respond) to angiogenesis inhibitor therapy.

Algorithms suitable for categorization of samples include but are not limited to k-nearest neighbor algorithms, support vector machines, linear discriminant analysis, diagonal linear discriminant analysis, updown, naive Bayesian algorithms, neural network algorithms, hidden Markov model algorithms, genetic algorithms, or any combination thereof.

When a binary classifier is compared with actual true values (e.g., values from a biological sample), there are typically four possible outcomes. If the outcome from a prediction is p (where “p” is a positive classifier output, such as the presence of a deletion or duplication syndrome) and the actual value is also p, then it is called a true positive (TP); however if the actual value is n then it is said to be a false positive (FP). Conversely, a true negative has occurred when both the prediction outcome and the actual value are n (where “n” is a negative classifier output, such as no deletion or duplication syndrome), and false negative is when the prediction outcome is n while the actual value is p. In one embodiment, consider a test that seeks to determine whether a person is likely or unlikely to respond to angiogenesis inhibitor therapy. A false positive in this case occurs when the person tests positive, but actually does respond. A false negative, on the other hand, occurs when the person tests negative, suggesting they are unlikely to respond, when they actually are likely to respond. The same holds true for classifying a lung cancer subtype.

The positive predictive value (PPV), or precision rate, or post-test probability of disease, is the proportion of subjects with positive test results who are correctly diagnosed as likely or unlikely to respond, or diagnosed with the correct lung cancer subtype, or a combination thereof. It reflects the probability that a positive test reflects the underlying condition being tested for. Its value does however depend on the prevalence of the disease, which may vary. In one example the following characteristics are provided: FP (false positive); TN (true negative); TP (true positive); FN (false negative). False positive rate (□)=FP/(FP+TN)-specificity; False negative rate (□)=FN/(TP+FN)-sensitivity; Power=sensitivity=1-□□; Likelihood-ratio positive=sensitivity/(l-specificity); Likelihood-ratio negative=(1-sensitivity)/specificity. The negative predictive value (NPV) is the proportion of subjects with negative test results who are correctly diagnosed.

In some embodiments, the results of the biomarker level analysis of the subject methods provide a statistical confidence level that a given diagnosis is correct. In some embodiments, such statistical confidence level is at least about, or more than about 85%, 90%, 91%, 92%, 93%, 94%, 95%, 96%, 97%, 98%, 99% 99.5%, or more.

In some embodiments, the method further includes classifying the lung tissue sample as a particular lung cancer subtype based on the comparison of biomarker levels in the sample and reference biomarker levels, for example present in at least one training set. In some embodiments, the lung tissue sample is classified as a particular subtype if the results of the comparison meet one or more criterion such as, for example, a minimum percent agreement, a value of a statistic calculated based on the percentage agreement such as (for example) a kappa statistic, a minimum correlation (e.g., Pearson's correlation) and/or the like.

It is intended that the methods described herein can be performed by software (stored in memory and/or executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including Unix utilities, C, C++, Java™, Ruby, SQL, SAS®, the R programming language/software environment, Visual Basic™, and other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Some embodiments described herein relate to devices with a non-transitory computer-readable medium (also can be referred to as a non-transitory processor-readable medium or memory) having instructions or computer code thereon for performing various computer-implemented operations and/or methods disclosed herein. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

In some embodiments, a single biomarker, or from about 5 to about 10, from about 5 to about 15, from about 5 to about 20, from about 5 to about 25, from about 5 to about 30, from about 5 to about 35, from about 5 to about 40, from about 5 to about 45, from about 5 to about 50 biomarkers (e.g., as disclosed in Table 1A, Table 1B, Table 1C, Table 2, Table 3, Table 4, Table 5 and Table 6) is capable of classifying types and/or subtypes of lung cancer with a predictive success of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between. In some embodiments, any combination of biomarkers disclosed herein (e.g., in Table 1A, Table 1B, Table 1C, Table 2, Table 3, Table 4, Table 5 and Table 6 and sub-combinations thereof) can used to obtain a predictive success of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between.

In some embodiments, a single biomarker, or from about 5 to about 10, from about 5 to about 15, from about 5 to about 20, from about 5 to about 25, from about 5 to about 30, from about 5 to about 35, from about 5 to about 40, from about 5 to about 45, from about 5 to about 50 biomarkers (e.g., as disclosed in Table 1A, Table 1B, Table 1C, Table 2, Table 3, Table 4, Table 5 and Table 6) is capable of classifying lung cancer types and/or subtypes with a sensitivity or specificity of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between. In some embodiments, any combination of biomarkers disclosed herein can be used to obtain a sensitivity or specificity of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between.

In some embodiments, one or more kits for practicing the methods of the invention are further provided. The kit can encompass any manufacture (e.g., a package or a container) including at least one reagent, e.g., an antibody, a nucleic acid probe or primer, and/or the like, for detecting the biomarker level of a classifier biomarker. The kit can be promoted, distributed, or sold as a unit for performing the methods of the present invention. Additionally, the kits can contain a package insert describing the kit and methods for its use.

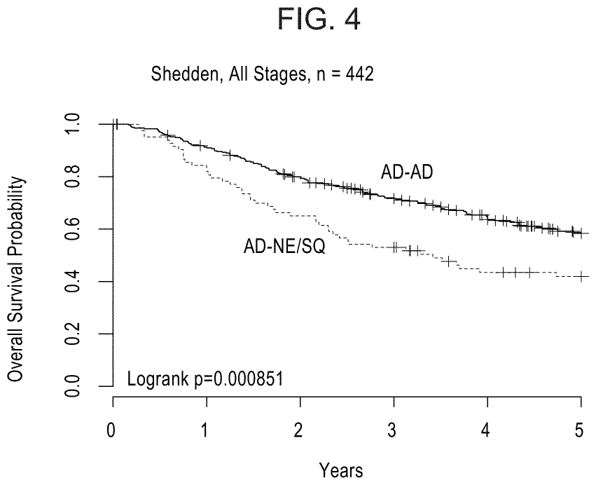

In one embodiment, a method is provided herein for determining a disease outcome or prognosis for a patient suffering from cancer. In some cases, the cancer is lung cancer. The method can comprise determining a disease outcome or prognosis for the patient by comparing a molecular subtype of the patient's cancer with a morphological subtype of the patient's cancer, whereby the presence or absence of concordance between the molecular and morphological subtypes predicts the disease outcome or prognosis of the patient. In one embodiment, discordance between the molecular subtype and the morphological subtype indicates a poor prognosis or poor disease outcome. The poor prognosis or disease outcome can be in comparison to a patient suffering from the same type of cancer (e.g., lung cancer) whose molecular and morphological subtype determinations are concordant. The disease outcome or prognosis can be measured by examining the overall survival for a period of time or intervals (e.g., 0 to 36 months or 0 to 60 months). In one embodiment, survival is analyzed as a function of subtype (e.g., for lung cancer, adenocarcinoma (TRU, PI, and PP), neuroendocrine (small cell carcinoma and carcinoid), or squamous). Relapse-free and overall survival can be assessed using standard Kaplan-Meier plots (see FIGS. 4-11) as well as Cox proportional hazards modeling.

In one embodiment, the molecular subtype is determined by detecting expression levels of classifier biomarkers, thereby obtaining an expression profile. The expression profile can be determined using any of the methods provided herein. In some cases, the patient is suffering from lung cancer and the molecular subtype of a lung tissue sample obtained from the patient is determined by detecting the levels of a single biomarker, or from about 5 to about 10, from about 5 to about 15, from about 5 to about 20, from about 5 to about 25, from about 5 to about 30, from about 5 to about 35, from about 5 to about 40, from about 5 to about 45, from about 5 to about 50 classifier biomarkers of Table 1A, Table 1B, Table 1C, Table 2, Table 3, Table 4, Table 5 or Table 6 using any of the methods provided herein for detecting the expression levels (e.g., RNA-seq, RT-PCR, or hybridization assay such as, for example, microarray hybridization assay).

In one embodiment, the molecular subtype is determined by detecting expression levels of at least five classifier biomarkers in Table 1A, Table 1B, Table 1C, Table 2, Table 3, Table 4, Table 5 or Table 6 at a nucleic acid level in a lung tissue sample by performing RT-PCR (or qRT-PCR) and comparing the detected expression levels to those of a reference sample or training set as described herein in order to determine if the molecular subtype of the lung tissue sample obtained from the patient is an adenocarcinoma, squamous cell carcinoma, or a neuroendocrine subtype. The neuroendocrine subtype can encompass small cell carcinoma and carcinoid. The adenocarcinoma subtype can be further classified as being TRU, PI, or PP. The RT-PCR can be performed with primers specific to the at least five classifier biomarkers. The primers specific for the at least five classifier biomarkers are forward and reverse primers listed in Table 1A, Table 1B, Table 1C, Table 2, Table 3, Table 4, Table 5 or Table 6.

In one embodiment, the molecular subtype is determined by probing the levels of at least five classifier biomarkers in Table 1A, Table 1B, Table 1C, Table 2, Table 3, Table 4, Table 5 or Table 6 at a nucleic acid level in a lung tissue sample by mixing the sample with five or more oligonucleotides that are substantially complementary to portions of nucleic acid molecules of the at least five classifier biomarkers of Table 1A, Table IB, Table 1C, Table 2, Table 3, Table 4, Table 5 or Table 6 under conditions suitable for hybridization of the five or more oligonucleotides to their complements or substantial complements, detecting whether hybridization occurred between the five or more oligonucleotides to their complements or substantial complements, obtaining hybridization values of the at least five classifier biomarkers based on the detecting step and comparing the detected hybridization values to those of a reference sample or training set as described herein in order to determine if the molecular subtype of the lung tissue sample obtained from the patient is an adenocarcinoma, squamous cell carcinoma, or a neuroendocrine subtype. The neuroendocrine subtype can encompass small cell carcinoma and carcinoid. The adenocarcinoma subtype can be further classified as being TRU, PI, or PP.

In one embodiment, the morphological subtype of a tissue sample (e.g., lung tissue sample) is a histological analysis. Histological analysis can be performed using any of the methods known in the art. In one embodiment, a lung tissue sample is assigned a histological subtype of adenocarcinoma, squamous, or neuroendocrine based on the histological analysis. In one embodiment, the histological subtype of a lung tissue sample obtained from a patient suffering from lung cancer is compared to the molecular subtype of the lung tissue sample, whereby the molecular subtype is determined by examining gene expression levels of classifier genes (e.g. from Table 1A, Table IB, Table 1C, Table 2, Table 3, Table 4, Table 5 or Table 6). In one embodiment, the histological subtype and molecular subtypes are in concordance, whereby the overall survival of the patient (as determined for example by using standard Kaplan-Meier plots as well as Cox proportional hazards modeling) is substantially similar to the overall survival of other patients with the same subtype of cancer. In one embodiment, the histological subtype and molecular subtype are discordant, whereby the overall survival of the patient (as determined for example by using standard Kaplan-Meier plots as well as Cox proportional hazards modeling) is substantially dissimilar to the overall survival of other patients with concordant molecular and histological subtype determinations of cancer. The overall survival probability of patient's with discordant subtypes can be 5%, 10%, 15%, 20%, 25%, 30%, 35%, 40%, 45%, 50%, 60%, 70%, 75%, 80%, 85%, 90%, 95%, 97.5%, 99%, 99.5%, or 99.9% less or lower than the overall survival probability of patient's with concordant subtypes of cancer (e.g., lung cancer).

In one embodiment, upon determining a patient's lung cancer subtype, the patient is selected for suitable therapy, for example chemotherapy or drug therapy with an angiogenesis inhibitor. In one embodiment, the therapy is angiogenesis inhibitor therapy, and the angiogenesis inhibitor is a vascular endothelial growth factor (VEGF) inhibitor, a VEGF receptor inhibitor, a platelet derived growth factor (PDGF) inhibitor or a PDGF receptor inhibitor.

In another embodiment, the angiogenesis inhibitor is an integrin antagonist, a selectin antagonist, an adhesion molecule antagonist (e.g., antagonist of intercellular adhesion molecule (ICAM)-1, ICAM-2, ICAM-3, platelet endothelial adhesion molecule (PCAM), vascular cell adhesion molecule (VCAM)), lymphocyte function-associated antigen 1 (LFA-1)), a basic fibroblast growth factor antagonist, a vascular endothelial growth factor (VEGF) modulator, or a platelet derived growth factor (PDGF) modulator (e.g., a PDGF antagonist). In one embodiment of determining whether a subject is likely to respond to an integrin antagonist, the integrin antagonist is a small molecule integrin antagonist, for example, an antagonist described by Paolillo et al. (Mini Rev Med Chem, 2009, volume 12, pp. 1439-1446, incorporated by reference in its entirety), or a leukocyte adhesion-inducing cytokine or growth factor antagonist (e.g., tumor necrosis factor-α (TNF-α), interleukin-1β (IL-1β), monocyte chemotactic protein-1 (MCP-1) and a vascular endothelial growth factor (VEGF)), as described in U.S. Pat. No. 6,524,581, incorporated by reference in its entirety herein.

The methods provided herein are also useful for determining whether a subject is likely to respond to one or more of the following angiogenesis inhibitors: interferon gamma 1β, interferon gamma 1β (Actimmune®) with pirfenidone, ACUHTR028, αVβ5, aminobenzoate potassium, amyloid P, ANG1122, ANG1170, ANG3062, ANG3281, ANG3298, ANG4011, anti-CTGF RNAi, Aplidin, Astragalus membranaceus extract with Salvia and Schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon α-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide, VA999260, XV615, or a combination thereof.

In another embodiment, a method is provided for determining whether a subject is likely to respond to one or more endogenous angiogenesis inhibitors. In a further embodiment, the endogenous angiogenesis inhibitor is endostatin, a 20 kDa C-terminal fragment derived from type XVIII collagen, angiostatin (a 38 kDa fragment of plasmin), or a member of the thrombospondin (TSP) family of proteins. In a further embodiment, the angiogenesis inhibitor is a TSP-1, TSP-2, TSP-3, TSP-4 and TSP-5. Methods for determining the likelihood of response to one or more of the following angiogenesis inhibitors are also provided a soluble VEGF receptor, e.g., soluble VEGFR-1 and neuropilin 1 (NPR1), angiopoietin-1, angiopoietin-2, vasostatin, calreticulin, platelet factor-4, a tissue inhibitor of metalloproteinase (TIMP) (e.g., TIMP1, TIMP2, TIMP3, TIMP4), cartilage-derived angiogenesis inhibitor (e.g., peptide troponin I and chrondomodulin I), a disintegrin and metalloproteinase with thrombospondin motif 1, an interferon (IFN) (e.g., IFN-α, IFN-β, IFN-γ), a chemokine, e.g., a chemokine having the C-X-C motif (e.g., CXCL10, also known as interferon gamma-induced protein 10 or small inducible cytokine B10), an interleukin cytokine (e.g., IL-4, IL-12, IL-18), prothrombin, antithrombin III fragment, prolactin, the protein encoded by the TNFSF15 gene, osteopontin, maspin, canstatin, proliferin-related protein.

In one embodiment, a method for determining the likelihood of response to one or more of the following angiogenesis inhibitors is provided is angiopoietin-1, angiopoietin-2, angiostatin, endostatin, vasostatin, thrombospondin, calreticulin, platelet factor-4, TIMP, CDAI, interferon α, interferon β, vascular endothelial growth factor inhibitor (VEGI) meth-1, meth-2, prolactin, VEGI, SPARC, osteopontin, maspin, canstatin, proliferin-related protein (PRP), restin, TSP-1, TSP-2, interferon gamma 1β, ACUHTR028, αVβ5, aminobenzoate potassium, amyloid P, ANG1122, ANG1170, ANG3062, ANG3281, ANG3298, ANG4011, anti-CTGF RNAi, Aplidin, Astragalus membranaceus extract with Salvia and Schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon α-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide, VA999260, XV615 or a combination thereof.

In yet another embodiment, a methods for determining the likelihood of response to one or more of the following angiogenesis inhibitors is provided: pazopanib (Votrient), sunitinib (Sutent), sorafenib (Nexavar), axitinib (Inlyta), ponatinib (Iclusig), vandetanib (Caprelsa), cabozantinib (Cometrig), ramucirumab (Cyramza), regorafenib (Stivarga), ziv-aflibercept (Zaltrap), or a combination thereof. In yet another embodiment, the angiogenesis inhibitor is a VEGF inhibitor. In a further embodiment, the VEGF inhibitor is axitinib, cabozantinib, aflibercept, brivanib, tivozanib, ramucirumab or motesanib. In yet a further embodiment, the angiogenesis inhibitor is motesanib.

In one embodiment, the methods provided herein relate to determining a subject's likelihood of response to an antagonist of a member of the platelet derived growth factor (PDGF) family, for example, a drug that inhibits, reduces or modulates the signaling and/or activity of PDGF-receptors (PDGFR). For example, the PDGF antagonist, in one embodiment, is an anti-PDGF aptamer, an anti-PDGF antibody or fragment thereof, an anti-PDGFR antibody or fragment thereof, or a small molecule antagonist. In one embodiment, the PDGF antagonist is an antagonist of the PDGFR-α or PDGFR-β. In one embodiment, the PDGF antagonist is the anti-PDGF-β aptamer E10030, sunitinib, axitinib, sorefenib, imatinib, imatinib mesylate, nintedanib, pazopanib HCl, ponatinib, MK-2461, dovitinib, pazopanib, crenolanib, PP-121, telatinib, imatinib, KRN 633, CP 673451, TSU-68, Ki8751, amuvatinib, tivozanib, masitinib, motesanib diphosphate, dovitinib dilactic acid, linifanib (ABT-869).

### EXAMPLES

The present invention is further illustrated by reference to the following Examples. However, it should be noted that these Examples, like the embodiments described above, is illustrative and is not to be construed as restricting the scope of the invention in any way.

### Example 1

### Methods to Validate a 57 Gene Expression Lung Subtype Panel (LSP)

Several publically available lung cancer gene expression data sets including 2,168 lung cancer samples (TCGA, NCI, UNC, Duke, Expo, Seoul, Tokyo, and France) were assembled to validate a 57 gene expression Lung Subtype Panel (LSP) developed to complement morphologic classification of lung tumors. LSP included 52 lung tumor classifying genes plus 5 housekeeping genes. Data sets with both gene expression data and lung tumor morphologic classification were selected. Three categories of genomic data were represented in the data sets: Affymetrix U133+2 (n=883) (also referred to as “A-833”), Agilent 44K (n=334) (also referred to as “A-334”), and Illumina RNAseq (n=951) (also referred to as “I-951”). Data sources are provided in Table 7 and normalization methods in Table 8. Samples with a definitive diagnosis of adenocarcinoma, carcinoid, small cell, and squamous cell carcinoma were used in the analysis.

TABLE 7
Data sources for publicly available
lung cancer gene expression data

| Source | Platform(s) | N | Subtype | Ref |
|---|---|---|---|---|
| TCGA¹ | RNASeq | 528 | adenocarcinomas | TCGA-DCC |
| | (LUAD) | | | |
| TCGA² | RNASeq | 534 | Squamous | TCGA-DCC |
| | (LUSC) | | | |
| UNC³ | Agilent_44K | 56 | 56 squamous | CCR (2010) |
| | | | | PMID: 20643781 |
| UNC⁴ | Agilent_44K | 116 | 116 | PLoS One (2012) |
| | | | adenocarcinomas | PMID: 22590557 |
| NCI⁵ | Agilent_44K | 172 | 56 adenocarcinoma, | CCR (2009) |
| | | | 92 squamous, 10 | |
| | | | large cell | |
| Korea⁶ | HG-U133 + 2 | 138 | 63 adenocarcinoma, | CCR (2008) |
| | | | 75 squamous | PMID: 19010856 |
| Expo⁷ | HG-U133 + 2 | 130 | all histology | GSE2109 |
| | | | subtypes | |
| French⁸ | HG-U133 + 2 | 307 | all histology | Sci Transl Med |
| | | | subtypes | (2013) |
| | | | | PMID: 23698379 |
| Duke⁹ | HG-U133 + 2 | 118 | adenocarcinoma and | Nature (2006) |
| | | | squamous | PMID: 16273092 |
| Tokyo¹⁰ | HG-U133 + 2 | 246 | adenocarcinomas | PLoS One (2012) |
| | | | | PMID: 22080568, |
| | | | | 74078470 |

¹https://tcga-data.nci.nih.gov/tcgafiles/ftp_auth/distro_ftpusers/anonymous/tumor/luad/cgcc/unc.edu/illuminahiseq_rnaseqv2/maseqv2/?C=S;O=A
²https://tcga-data.nci.nih.gov/tcgafiles/ftp_auth/distro_ftpusers/anonymous/tumor/lusc/cgcc/unc.edu/illuminahiseq_rnaseqv2/maseqv2/
³http://www.ncbi.nlm.nih.gov/geo/query/acc.cgi?acc=GSE17710
⁴http://www.ncbi.nlm.nih.gov/geo/query/acc.cgi?acc=GSE26939
⁵http://research.agendia.com/
⁶http://www.ncbi.nlm.nih.gov/geo/query/acc.cgi?acc=GSE8894
⁷http://www.ncbi.nlm.nih.gov/geo/query/acc.cgi?acc=GSE2109
⁸http://www.ncbi.nlm.nih.gov/geo/query/acc.cgi?acc=GSE30219
⁹http://www.ncbi.nlm.nih.gov/geo/query/acc.cgi?acc=GSE3141
¹⁰http://www.ncbi.nlm.nih.gov/geo/query/acc.cgi?acc=GSE31210

TABLE 8
Normalization methods used for the
3 public gene expression datasets

| Source | Platforms | Data Preprocessing/Normalization |
|---|---|---|
| TCGA | RNASeq | RSEM expression estimates are normalized |
| | | to set the upper quartile count at 1000 |
| | | for gene level, 2 based log transformed, |
| | | data matrix is row (gene) median |
| | | centered, column (sample) standardized. |
| UNC + | Agilent_44K | 2 based log ratio of the two channel |
| NKI | | intensities are LOWESS normalized, |
| | | data matrix is row (gene) median |
| | | centered, column (sample) standardized. |
| Affy | HG-U133 + | MAS5 normalized one channel intensities |
| | 2 | are 2 based log transformed, data matrix |
| | | is row (gene) median centered, |
| | | column (sample) standardized. |

The A-833 dataset was used as training for calculation of adenocarcinoma, carcinoid, small cell carcinoma, and squamous cell carcinoma gene centroids according to methods described previously. Gene centroids trained on the A-833 data were then applied to the normalized TCGA and A-334 datasets to investigate LSP's ability to classify lung tumors using publicly available gene expression data. For the application of A-833 training centroids to the A-833 dataset, evaluation was performed using Leave One Out (LOO) cross validation. Spearman correlations were calculated for tumor sample gene expression results to the A-833 gene expression training centroids. Tumors were assigned a genomic-defined histologic type (carcinoid, small cell, adenocarcinoma and squamous cell carcinoma) corresponding to the maximally correlated centroids. A 2 class, 3 class, and 4 class prediction was explored. Correct predictions were defined as LSP calls matching the tumor's histologic diagnosis. Percent agreement was defined as the number of correct predictions divided by the number of all predictions and an agreement kappa statistic was calculated.

Ten lung tumor RNA expression datasets were combined into three platform specific data sets (A-833, A-334, and I-951). The patient population was diverse and included smokers and nonsmokers with tumors ranging from Stage 1-Stage IV. Sample characteristics and lung cancer diagnoses of the three datasets are included in Table 9.

TABLE 9
Sample Characteristics

| | TCGA RNA | | |
|---|---|---|---|
| Characteristic | Seq | Agilent | Affymetrix |
| Total # of samples | 1062 | 334 | 875 |
| Tumor specimen histology | | | |
| Adenocarcinoma | 468 | 174 | 490 |
| Carcinoid | 0 | 0 | 23 |
| Small cell carcinoma | 0 | 0 | 24 |
| Neuroendocrine (NOS) | 0 | 0 | 6 |
| Squamous Cell Carcinoma | 483 | 148 | 227 |
| Other (excluded from | 111 | 12 | 105 |
| analysis) | | | |
| Gender | | | |
| Female/Male/NA | 285/366/300 | 87/85/150 | 272/491/7 |
| Age at Diagnosis | | | |
| Median (range) | 67/(38-88) | 66/(37-90) | 63/(13-85) |
| Age not available | 323 | 150 | 7 |
| Stage | | | |
| I | 355 | NA | NA |
| II | 146 | NA | NA |
| III | 119 | NA | NA |
| IV | 26 | NA | NA |
| Stage not available | 305 | 322 | 770 |
| Smoking | | | |
| Smoker | 386 | NA | NA |
| Nonsmoker | 39 | NA | NA |
| Smoking status not | 526 | 322 | 770 |
| available | | | |

Predicted tumor type for a 2 class, 3 class, and 4 class predictor were compared with tumor morphologic classification and percent agreement and Fleiss' kappa was calculated for each predictor (Tables 10a-c).

TABLE 10a
A-833 dataset training gene centroids applied to 2 other publicly available lung
cancer gene expression databases (TCGA & A-334) for a 2 class prediction of
lung tumor type. LOO cross validation was performed for the A-833 dataset.

| | Prediction | | |
|---|---|---|---|
| | TCGA RNAseq | Agilent | Affymetrix LOO |
| Histology Diagnosis | AD ∥ SQ ∥ Sum | AD ∥ SQ ∥ Sum | AD ∥ SQ ∥ Sum |
| Adenocarcinoma (AD) | 452 ∥ 16 ∥ 468 | 151 ∥ 23 ∥ 174 | 423 ∥ 67 ∥ 490 |
| Squamous cell carcinoma (SQ) | 37 ∥ 446 ∥ 483 | 39 ∥ 109 ∥ 148 | 41 ∥ 186 ∥ 227 |
| Sum | 489 ∥ 462 ∥ 951 | 190 ∥ 132 ∥ 322 | 464 ∥ 253 ∥ 717 |
| % Agreement | 94% | 81% | 85% |
| Kappa | 0.89 | 0.61 | 0.66 |

TABLE 10b
A-833 dataset training gene centroids applied to data from 2 other publicly available
lung cancer gene expression databases (TCGA & A-334) for a 3 class prediction
of lung tumor type. LOO cross validation was performed for the A-833 dataset.

| | Prediction | | |
|---|---|---|---|
| | TCGA RNAseq | Agilent | Affymetrix LOO |
| Histology Diagnosis | AD ∥ NE ∥ SQ ∥ Sum | AD ∥ NE ∥ SQ∥ Sum | AD ∥ NE ∥ SQ ∥ Sum |
| Adenocarcinoma (AD) | 419 ∥ 29 ∥ 20 ∥ 468 | 141 ∥ 6 ∥ 27 ∥ 174 | 399 ∥ 3 ∥ 88 ∥ 490 |
| Neuroendocrine (NE) | NA ∥ NA ∥ NA ∥ NA | NA ∥ NA ∥ NA ∥ NA | 2 ∥ 49 ∥ 2 ∥ 53 |
| Squamous cell carcinoma (SQ) | 23 ∥ 15 ∥ 445 ∥ 483 | 28 ∥ 3 ∥ 117 ∥ 148 | 25 ∥ 7 ∥ 195 ∥ 227 |
| Sum | 442 ∥ 44 ∥ 465 ∥ 951 | 169 ∥ 9 ∥ 144 ∥ 322 | 426 ∥ 59 ∥ 285 ∥ 770 |
| % Agreement | 91% | 80% | 84% |
| Kappa | 0.82 | 0.61 | 0.69 |

TABLE 10c
A-833 dataset training gene centroids applied to data from 2 other publicly available lung cancer gene expression databases
(TCGA & A-334) for a 4 class prediction of lung tumor type. L00 cross validation was performed for the A-833 dataset.

| | Prediction | | | | | | | | | | | | | | |
|---|---|---|---|---|---|---|---|---|---|---|---|---|---|---|---|
| | TCGA RNAseq | | | | | Agilent | | | | Affymetrix LOO | | | | | |
| Histology Diagnosis | AD | CA | SC | SQ | Sum | AD | CA | SC | SQ | Sum | AD | CA | SC | SQ | Sum |
| Adenocarcinoma (AD) | 428 | 2 | 20 | 18 | 468 | 138 | 2 | 5 | 29 | 174 | 389 | 1 | 3 | 97 | 490 |
| Carcinoid (CA) | NA | NA | NA | NA | NA | NA | NA | NA | NA | NA | 1 | 22 | 0 | 0 | 23 |
| Small Cell (SC) | NA | NA | NA | NA | NA | NA | NA | NA | NA | NA | 1 | 1 | 20 | 2 | 24 |
| Squamous cell | 23 | 2 | 15 | 443 | 483 | 27 | 0 | 3 | 118 | 148 | 27 | 1 | 5 | 194 | 227 |
| carcinoma (SQ) | | | | | | | | | | | | | | | |
| Sum | 451 | 4 | 35 | 461 | 951 | 165 | 2 | 8 | 147 | 322 | 418 | 25 | 28 | 293 | 764 |

| | | | |
|---|---|---|---|
| % Agreement | 92% | 80% | 82% |
| kappa | 0.84 | 0.60 | 0.65 |

Evaluation of inter-observer reproducibility of lung cancer diagnosis based on morphologic classification alone has previously been published. Overall inter-observer agreement improved with simplification of the typing scheme. Using the comprehensive 2004 World Health Organization classification system inter-observer agreement was low (k=0.25). Agreement improved with simplification of the diagnosis to the therapeutically relevant 2 type differentiation of squamous/non-squamous (k=0.55). Agreement of inter-observer diagnosis is compared to agreement of 2, 3 and 4 class LSP diagnosis in this validation study (Table 11).

TABLE 11
Inter-observer agreement (3) measured using kappa statistic and LSP agreement
with histologic diagnosis in multiple gene expression datasets.

| | WHO 2004 | 2 Class Squamous/Nonsquamous | | | |
|---|---|---|---|---|---|
| | Classification | cell carcinoma | | 3 Class | 4 Class |
| | Inter- | Inter- | LSP | LSP | LSP |
| | observer | observer | Agreement | Agreement | Agreement |
| Agreement | Agreement | Agreement | w/Hist DX | w/Hist DX | w/Hist DX |
| kappa | 0.25 | 0.55 | 0.61-0.89 | 0.61-0.82 | 0.60-0.84 |

Differentiation among various morphologic subtypes of lung cancer is increasingly important as therapeutic development and patient management become more specifically targeted to unique features of each tumor. Histologic diagnosis can be challenging and several studies have demonstrated limited reproducibility of morphologic diagnoses. The addition of several immunohistochemistry markers, such as p63 and TTF-1 improves diagnostic precision but many lung cancer biopsies are limited in size and/or cellularity precluding full characterization using multiple IHC markers. Agreement was markedly better for all the classifiers (2, 3, and 4 type) in the TCGA RNAseq dataset (% agreement range 91%-94%) as compared to the other datasets possibly due to the greater accuracy of the histologic diagnosis and/or the greater precision of the RNA expression results. Despite several limitations described below, this study demonstrates that LSP, can be a valuable adjunct to histology in typing lung tumors.

In multiple datasets with hundreds of lung cancer samples, molecular profiling using the Lung Subtype Panel (LSP) compared favorably to light microscopic derived diagnoses, and showed a higher level of agreement than pathologist reassessments. RNA-based tumor subtyping can provide valuable information in the clinic, especially when tissue is limiting and the morphologic diagnosis remains unclear.

The disclosures of the following references are incorporated herein by reference in their entireties for all purposes:
a. American Cancer Society. Cancer Facts and Figures, 2014.
b. National Comprehensive Cancer Network (NCCN) Clinical Practice Guideline in Oncology. Non-Small Cell Lung Cancer. Version 2.2013.
c. Grilley Olson J E, Hayes D N, Moore D T, et al. Arch Pathol Lab Med 2013; 137: 32-40
d. Thunnissen E, Boers E, Heideman D A, et al. Virchows Arch 2012; 461:629-38.
e. Wilkerson M D, Schallheim J M, Hayes D N, et al. J Molec Diagn 2013; 15:485-497.
f. Li B, Dewey C N. BMC Bioinformatics 2011, 12:323 doi:10.1186/1471-2105-12-323
g. Yang Y H, Dudoit S, Luu P, et al. Nucleic Acids Research 2002, 30:e15.
h. Hubbell E, Liu, W, Mei R. Bioinformatics (2002) 18 (12): 1585-1592. doi:10.1093/bioinformatics/18.12.1585.
i. Travis W D, Brambilla E, Muller-Hermelink H K, Harris C C. Pathology and Genetics of Tumors of the Lung, Pleura, Thymus, and Heart. 3rd ed. Lyon, France: IARC Press; 2004. World Health Organization Classification of Tumors: vol 10.
j. Travis W D and Rekhtman N. Sem Resp and Crit Care Med 2011; 32(1): 22-31.

### Example 2

### Lung Cancer Subtyping of Multiple Fresh Frozen and Formalin Fixed Paraffin Embedded Lung Tumor Gene Expression Datasets

Multiple datasets comprising 2,177 samples were assembled to evaluate a Lung Subtype Panel (LSP) gene expression classifier. The datasets included several publically available lung cancer gene expression data sets, including 2,099 Fresh Frozen lung cancer samples (TCGA, NCI, UNC, Duke, Expo, Seoul, and France) as well as newly collected gene expression data from 78 FFPE samples. Data sources are provided in the Table 12 below. The 78 FFPE samples were archived residual lung tumor samples collected at the University of North Carolina at Chapel Hill (UNC-CH) using an IRB approved protocol. Only samples with a definitive diagnosis of AD, carcinoid, Small Cell Carcinoma (SCC), or SQC were used in the analysis. A total of 4 categories of genomic data were available for analysis: Affymetrix U133+2 (n=693), Agilent 44K (n=344), Illumina® RNAseq (n=1,062) and newly collected qRT-PCR (n=78) data.

Archived FFPE lung tumor samples (n=78) were analyzed using a qRT-PCR gene expression assay as previously described (Wilkerson et al. J Molec Diagn 2013; 15:485-497, incorporated by reference herein in its entirety for all purposes) with the following modifications. RNA was extracted from one 10 μm section of FFPE tissue using the High Pure RNA Paraffin Kit (Roche Applied Science, Indianapolis, Ind.). Extracted RNA was diluted to 5 ng/μL and first strand cDNA was synthesized using gene specific 3′ primers in combination with random hexamers (Superscript III®, Invitrogen®, Thermo Fisher Scientific Corp, Waltham, Mass.). An ABI 7900 (Applied Biosystems, Thermo Fisher Scientific Corp, Waltham, Mass.) was used for qRT-PCR with continuous SYBR green fluorescence (530 nm) monitoring. ABI 7900 quantitation software generated amplification curves and associated threshold cycle (Ct) values. Original clinical diagnoses gathered with the samples is in Table 13.

TABLE 12

| Source | Platforms | N | Subtype | Normalization Method Used | Data Source |
|---|---|---|---|---|---|
| TCGA | RNASeq | 528 | adenocarcinomas | RSEM expression estimates are | Ref 16 |
| | (LUAD) | | | normalized to set the upper | TCGA |
| TCGA | RNASeq | 534 | Squamous cell | quartile count at 1000 for gene | Ref 15 |
| | (LUSC) | | carcinoma | level, 2 based log transformed, | TCGA |
| | | | | data matrix is row (gene) median | |
| | | | | centered, column (sample) | |
| | | | | standardized²⁸ | |
| UNC | Agilent_44K | 56 | Squamous cell | 2 based log ratio of the two | Ref 19 |
| | | | carcinoma | channel intensities are LOWESS | GSE |
| | | | | normalized, data matrix is row | 17710 |
| UNC | Agilent_44K | 116 | adenocarcinomas | (gene) median centered, column | Ref 20 |
| | | | | (sample) standardized²⁹ | GSE26939 |
| NCI | Agilent_44K | 172 | Adenocarcinoma, | | Ref 22 |
| | | | squamous cell, & | | http://research.agendia.com/ |
| | | | large cell | | |
| Korea | HG-U133 + 2 | 138 | Adenocarcinoma, | MASS normalized one channel | Ref 23 |
| | | | squamous cell | intensities are 2 based log | GSE8894 |
| | | | carcinoma | transformed, data matrix is row | |
| Expo | HG-U133 + 2 | 130 | All histology | (gene) median centered, column | Ref 24 |
| | | | subtypes | (sample) standardized³⁰ | GSE2109 |
| French | HG-U133 + 2 | 307 | All histology | | Ref 25 |
| | | | subtypes | | GSE30219 |
| Duke | HG-U133 + 2 | 118 | Adenocarcinoma, | | Ref 26 |
| | | | squamous cell | | GSE3141 |
| | | | carcinoma | | |
| UNC | FFPE tissue | 78 | Adenocarcinoma, | FFPE sample gene expression | Ref 27 |
| | RT-PCR | | squamous cell | data was scaled to align gene | Supplment |
| | | | carcinoma, small | variance with Wilkerson et al. | al File #1 |
| | | | cell & carcinoid | data²¹. A gene-specific scaling | |
| | | | | factor was calculated that took | |
| | | | | into account label frequency | |
| | | | | differences between the data sets. | |

TABLE 13

| Sample | Label |
|---|---|
| VELO001 | Squamous.Cell.Carcinoma |
| VELO002 | Squamous.Cell.Carcinoma |
| VELO004 | Adenocarcinoma |
| VELO006 | Squamous.Cell.Carcinoma |
| VELO007 | Squamous.Cell.Carcinoma |
| VELO008 | Squamous.Cell.Carcinoma |
| VELO010 | Squamous.Cell.Carcinoma |
| VELO011 | Squamous.Cell.Carcinoma |
| VELO012 | Squamous.Cell.Carcinoma |
| VELO013 | Squamous.Cell.Carcinoma |
| VELO014 | Squamous.Cell.Carcinoma |
| VELO015 | Adenocarcinoma |
| VELO016 | Squamous.Cell.Carcinoma |
| VELO017 | Squamous.Cell.Carcinoma |
| VELO018 | Squamous.Cell.Carcinoma |
| VELO019 | Squamous.Cell.Carcinoma |
| VELO020 | Adenocarcinoma |
| VELO021 | Adenocarcinoma |
| VELO022 | Adenocarcinoma |
| VELO023 | Adenocarcinoma |
| VELO024 | Adenocarcinoma |
| VELO025 | Adenocarcinoma |
| VELO026 | Adenocarcinoma |
| VELO027 | Adenocarcinoma |
| VELO028 | Adenocarcinoma |
| VELO029 | Adenocarcinoma |
| VELO030 | Adenocarcinoma |
| VELO031 | Adenocarcinoma |
| VELO032 | Adenocarcinoma |
| VELO033 | Adenocarcinoma |
| VELO034 | Adenocarcinoma |
| VELO035 | Adenocarcinoma |
| VELO036 | Adenocarcinoma |
| VELO037 | Adenocarcinoma |
| VELO038 | Squamous.Cell.Carcinoma |
| VELO039 | Squamous.Cell.Carcinoma |
| VELO040 | Squamous.Cell.Carcinoma |
| VELO042 | Squamous.Cell.Carcinoma |
| VELO044 | Squamous.Cell.Carcinoma |
| VELO046 | Squamous.Cell.Carcinoma |
| VELO048 | Squamous.Cell.Carcinoma |
| VELO049 | Squamous.Cell.Carcinoma |
| VELO050 | Adenocarcinoma |
| VELO041 | Squamous.Cell.Carcinoma |
| VELO043 | Squamous.Cell.Carcinoma |
| VELO045 | Squamous.Cell.Carcinoma |
| VELO055 | Neuroendocrine |
| VELO056 | Neuroendocrine |
| VELO057 | Neuroendocrine |
| VELO058 | Neuroendocrine |
| VELO059 | Neuroendocrine |
| VELO060 | Neuroendocrine |
| VELO061 | Neuroendocrine |
| VELO062 | Neuroendocrine |
| VELO063 | Neuroendocrine |
| VELO064 | Neuroendocrine |
| VELO065 | Neuroendocrine |
| VELO066 | Neuroendocrine |
| VELO067 | Neuroendocrine |
| VELO068 | Neuroendocrine |
| VELO069 | Neuroendocrine |
| VELO070 | Neuroendocrine |
| VELO071 | Neuroendocrine |
| VELO072 | Neuroendocrine |
| VELO073 | Neuroendocrine |
| VELO074 | Neuroendocrine |
| VELO075 | Neuroendocrine |
| VELO076 | Neuroendocrine |
| VELO077 | Neuroendocrine |
| VELO078 | Neuroendocrine |
| VELO079 | Neuroendocrine |
| VELO080 | Neuroendocrine |
| VELO081 | Neuroendocrine |
| VELO082 | Neuroendocrine |
| VELO083 | Neuroendocrine |
| VELO084 | Neuroendocrine |
| VELO085 | Neuroendocrine |

Pathology review was only possible for the FFPE lung tumor cohort in which additional sections were collected and imaged. Two contiguous sections from each sample were Hematoxylin & Eosin (H&E) stained and scanned using an Aperio™ ScanScope® slide scanner (Aperio Technologies, Vista, Calif.). Virtual slides were viewable at magnifications equivalent to 32 to 320 objectives (340 magnifier). Pathologist review was blinded to the original clinical diagnosis and to the gene expression-based subtype classification. Pathology review-based histological subtype calls were compared to the original diagnosis (n=78). Agreement of pathology review was defined as those samples for which both slides were assigned the same subtype as the original diagnosis.

All statistical analyses were conducted using R 3.0.2 software (http://cran.R-project.org). Data analyses were conducted separately for FF and for FFPE tumor samples.

Fresh Frozen Dataset Analysis: Datasets were normalized as described in Table 12. The Affymetrix dataset served as the training set for calculation of AD, carcinoid, SCC, and SQC gene centroids according to methods described previously (Wilkerson et al. PLoS ONE. 2012; 7(5) e36530. Doi:10.1371/journal.pone.0036530; Wilkerson et al. J Molec Diagn 2013; 15:485-497, each of which is incorporated by reference herein in its entirety for all purposes)

Affymetrix training gene centroids are provided in Table 14. The training set gene centroids were tested in normalized TCGA RNAseq gene expression and Agilent microarray gene expression data sets. Due to missing data from the public Agilent dataset, the Agilent evaluations were performed with a 47 gene classifier, rather than a 52 gene panel with exclusion of the following genes: CIB1 FOXH1, LIPE, PCAM1, TUBA1.

TABLE 14

| | Adenocar- | Neuroen- | |
|---|---|---|---|
| Gene | cinoma | docrine | Squamous.Cell.Carcinoma |
| ABCC5 | −0.453 | 0.3715 | 1.1245 |
| ACVR1 | 0.0475 | 0.3455 | −0.0465 |
| ALDH3B1 | 0.4025 | −0.638 | −0.401 |
| ANTXR1 | −0.0705 | −0.478 | 0.014 |
| BMP7 | −0.532 | −0.6265 | 0.6245 |
| CACNB1 | 0.024 | 0.157 | −0.039 |
| CAPG | 0.109 | −1.9355 | −0.0605 |
| CBX1 | −0.2045 | 0.745 | 0.187 |
| CDH5 | 0.391 | 0.145 | −0.352 |
| CDKN2C | −0.0045 | 1.496 | 0.004 |
| CHGA | −0.143 | 5.7285 | 0.1075 |
| CIB1 | 0.1955 | −0.261 | −0.065 |
| CLEC3B | 0.449 | 0.6815 | −0.3085 |
| CYB5B | 0.058 | 1.487 | −0.03 |
| DOK1 | 0.233 | −0.355 | −0.183 |
| DSC3 | −0.781 | −0.8175 | 4.3445 |
| FEN1 | −0.5025 | −0.0195 | 0.4035 |
| FOXH1 | −0.0405 | 0.1315 | −0.0105 |
| GJB5 | −1.388 | −1.5505 | 0.7685 |
| HOXD1 | 0.17 | −0.462 | −0.288 |
| HPN | 0.5335 | 0.444 | −0.736 |
| HYAL2 | 0.1775 | 0.073 | −0.143 |
| ICA1 | 0.3455 | 1.048 | −0.233 |
| ICAM5 | 0.13 | −0.145 | −0.12 |
| INSM1 | 0.0705 | 7.5695 | −0.0245 |
| ITGA6 | −0.709 | 0.029 | 1.074 |
| LGALS3 | 0.1805 | −1.1435 | −0.2305 |
| LIPE | 0.0065 | 0.5225 | −0.0015 |
| LRP10 | 0.2565 | −0.087 | −0.16 |
| MAPRE3 | −0.0245 | 0.6445 | −0.0025 |
| ME3 | 0.3085 | 0.3415 | −0.2915 |
| MGRN1 | 0.429 | 0.8075 | −0.3775 |
| MYBPH | 0.04 | −0.193 | −0.054 |
| MYO7A | 0.083 | −0.287 | −0.109 |
| NFIL3 | −0.332 | −1.0425 | 0.3095 |
| PAICS | −0.2145 | 0.3915 | 0.2815 |
| PAK1 | −0.112 | 0.6095 | 0.0965 |
| PCAM1 | 0.232 | −0.256 | −0.144 |
| PIK3C2A | 0.1505 | 0.597 | −0.021 |
| PLEKHA6 | 0.4465 | 2.0785 | −0.2615 |
| PSMD14 | −0.251 | 0.5935 | 0.1635 |
| SCD5 | −0.1615 | 0.06 | 0.13 |
| SFN | −0.789 | −3.026 | 0.91 |
| SIAH2 | −0.5795 | 0.1895 | 0.7175 |
| SNAP91 | −0.0255 | 3.818 | 0.003 |
| STMN1 | −0.0995 | 1.2095 | 0.1405 |
| TCF2 | 0.2835 | −0.5175 | −0.4665 |
| TCP1 | −0.1685 | 0.9815 | 0.1985 |
| TFAP2A | −0.374 | −0.5075 | 0.3645 |
| TITF1 | 1.482 | 0.1525 | −1.2755 |
| TRIM29 | −1.0485 | −1.318 | 1.379 |
| TUBA1 | 0.155 | 1.71 | −0.07 |

TABLE 15

| | Adenocar- | Neuroen- | |
|---|---|---|---|
| Gene | cinoma | docrine | Squamous.Cell.Carcinoma |
| ABCC5 | −1.105993 | 0.53584995 | 0.28498017 |
| ACVR1 | −0.1780792 | 0.27746814 | −0.1331305 |
| ALDH3B1 | 2.21915126 | −1.0930042 | 0.82709803 |
| ANTXR1 | 0.14704523 | −0.0027417 | −0.1000265 |
| CACNB1 | −0.2032444 | 0.36015235 | −0.7588385 |
| CAPG | 0.52784999 | −0.6495988 | −0.0218352 |
| CBX1 | −0.5905845 | −0.0461076 | −0.2776489 |
| CDH5 | −0.1546498 | 0.53564677 | −0.9166437 |
| CDKN2C | −1.8382992 | −0.1614815 | −0.7501799 |
| CHGA | −6.2702431 | 8.18090411 | −7.4497926 |
| CIB1 | 0.29948877 | −0.1804507 | 0.06141265 |
| CLEC3B | 0.1454466 | 0.86221597 | −0.6686516 |
| CYB5B | −0.1957799 | 0.13060667 | −0.2393801 |
| DOK1 | 0.03629227 | 0.03029676 | −0.2861762 |
| DSC3 | 0.76811006 | −2.2230482 | 4.45353398 |
| FEN1 | −0.4100344 | −0.774919 | 0.19244803 |
| FOXH1 | 1.36365962 | −1.1539159 | 1.86758359 |
| GJB5 | 2.19942372 | −3.2908475 | 4.00132739 |
| HOXD1 | −0.069692 | −0.3296808 | 0.50430984 |
| HPN | 0.62232864 | −0.0416111 | −0.5391064 |
| HYAL2 | 0.47459315 | −0.2332929 | −0.0080073 |
| ICA1 | −0.8108302 | 1.25305275 | −2.1742476 |
| ICAM5 | 2.12506546 | −2.2078991 | 2.89691121 |
| INSM1 | −2.4346556 | 1.92393374 | −1.9749654 |
| ITGA6 | −0.7881662 | 0.36443897 | 0.54978058 |
| LGALS3 | −0.8270046 | 0.79512054 | −0.9453521 |
| LIPE | −0.2519692 | 0.29291064 | −0.2216243 |
| LRP10 | 0.09504093 | 0.14082188 | −0.4042101 |
| MAPRE3 | −0.6806204 | 1.2417945 | −0.5496704 |
| ME3 | 0.17668171 | 0.67674964 | −1.581183 |
| MGRN1 | −0.0839601 | 0.35069923 | −0.6885404 |
| MYBPH | 0.73519429 | −0.9569161 | 1.14344753 |
| MYO7A | 0.58098661 | −0.2096425 | 0.0488886 |
| NFIL3 | 0.22274434 | −0.337858 | 0.66234639 |
| PAICS | −0.2423309 | −0.1863934 | 0.39037381 |
| PAK1 | −0.3803406 | 0.15627507 | 0.0677904 |
| PCAM1 | 0.03655586 | 0.32457357 | −0.6957339 |
| PIK3C2A | −0.3868824 | 0.56861416 | −0.6629455 |
| PLEKHA6 | −0.4007847 | 1.31002812 | −1.9802266 |
| PSMD14 | −0.5115938 | 0.27513479 | −0.2847234 |
| SCD5 | −0.4770619 | −0.4338812 | 0.56043153 |
| SFN | 0.35719248 | −1.4361124 | 2.34498532 |
| SIAH2 | −0.4222382 | −0.3853078 | 0.43237756 |
| SNAP91 | −5.5499562 | 4.65742276 | −2.5441741 |
| STMN1 | −1.4075058 | 0.49776156 | −1.017481 |
| TCF2 | 1.96819785 | −0.4121173 | −0.6555613 |
| TCP1 | −2.9255287 | 2.322428 | −2.3059797 |
| TFAP2A | 2.02528144 | −2.9053184 | 3.62844763 |
| TITF1 | 0.46476685 | −9.82E−05 | −1.7079242 |
| TRIM29 | −1.6554559 | −0.6463626 | 2.94818107 |
| TUBA1 | 1.77126501 | −2.0395783 | 1.58902579 |

Evaluation of the Affymetrix data was performed using Leave One Out (LOO) cross validation. Spearman correlations were calculated for tumor test sample to the Affymetrix gene expression training centroids. Tumors were assigned a genomic-defined histologic type (AD, SQC, or NE) corresponding to the maximally correlated centroids. Correct predictions were defined as LSP calls matching the tumor's original histologic diagnosis. Percent agreement was defined as the number of correct predictions divided by the number of total predictions and an agreement kappa statistic was calculated.

qRT-PCR from FFPE sample analysis: Previously published training centroids (Wilkerson et al. J Molec Diagn 2013; 15:485-497, incorporated by reference herein), calculated from qRT-PCR data of FFPE lung tumor samples, were cross-validated in this new sample set of qRT-PCR gene expression from FFPE lung tumor tissue. Wilkerson et al. AD and SQC centroids were used as published (Wilkerson et al. J Molec Diagn 2013; 15:485-497, incorporated by reference herein). Neuroendocrine gene centroids were calculated similarly using published gene expression data (n=130) (Wilkerson et al. J Molec Diagn 2013; 15:485-497, incorporated by reference herein). The Wilkerson et al. gene centroids (Wilkerson et al. J Molec Diagn 2013; 15:485-497, incorporated by reference herein) for the FFPE tissue evaluation are included in Table 15. FFPE sample gene expression data was scaled to align gene variance with Wilkerson et al. data. A gene-specific scaling factor was calculated that took into account label frequency differences between the data sets. Gene expression data was then median centered, sign flipped (high Ct=low abundance), and scaled using the gene specific scaling factor. Subtype was predicted by correlating each sample with the 3 subtype centroids and assignment of the subtype with the highest correlation centroid (Spearman correlation).

Ten lung tumor gene expression datasets including nine FF plus one new FFPE qRT-PCR gene expression dataset were combined into four platform-specific data sets (Affymetrix, Agilent, Illumina RNAseq, and qRT-PCR). For the datasets where clinical information was available, the patient population was diverse and included smokers and nonsmokers with tumors ranging from Stage 1-Stage IV. Sample characteristics and lung cancer diagnoses of the datasets used in this study are included in Table 16. After exclusion of samples without a definitive diagnosis of AD, SQC, SCC, or carcinoid, and exclusion of 1 FFPE sample that failed qRT-PCR analysis, the following samples were available for further data analysis: Affymetrix (n=538), Agilent (n=322), Illumina RNAseq (n=951) and qRT-PCR (n=77).

TABLE 16

| | TCGA RNA | | | UNC |
|---|---|---|---|---|
| Characteristic | seq | Agilent | Affymetrix | FFPE |
| Total # of samples | 1062 | 344 | 693 | 78 |
| Tissue Preservation | Fresh | Fresh | Fresh | FFPE |
| | Frozen | Frozen | Frozen | |
| Tumor specimen | | | | |
| histology | | | | |
| Adenocarcinoma | 468 | 174 | 264 | 21 |
| Carcinoid | 0 | 0 | 23 | 15 |
| Small Cell Carcinoma | 0 | 0 | 24 | 16 |
| Squamous Cell | 483 | 148 | 227 | 25 |
| Carcinoma | | | | |
| Other(excluded from | 111 | 22 | 155 | 01 |
| analysis) | | | | |
| Gender | | | | |
| Female/Male/NA | 285/366/300 | 87/85/150 | 151/386/1 | NA |
| Age at Diagnosis | | | | |
| Median/(Range) | 67/(38-88) | 66/(37-90) | 65/(13-85) | NA |
| Age not available | 323 | 0 | 2 | NA |
| Stage | | | | |
| I | 355 | NA | NA | NA |
| II | 146 | NA | NA | NA |
| III | 119 | NA | NA | NA |
| IV | 26 | NA | NA | NA |
| Stage not available | 305 | 322 | 538 | 77 |
| Smoking | | | | |
| Smoker | 386 | NA | NA | NA |
| Nonsmoker | 39 | NA | NA | NA |
| Smoking status not | 526 | 322 | 538 | 77 |
| available | | | | |

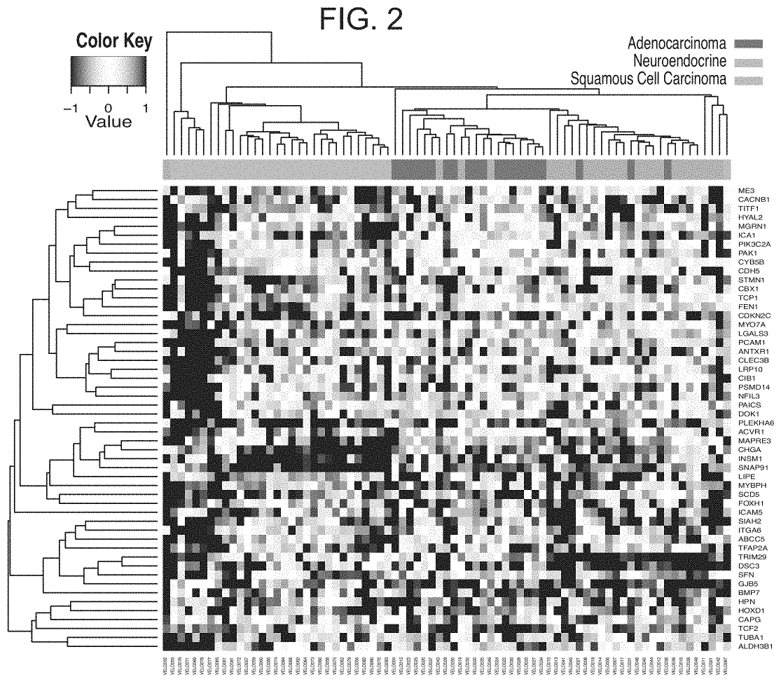

As a means of de novo evaluation of the new FFPE data set, we performed hierarchical clustering of LSP gene expression from the FFPE archived samples (n=77); as expected, this analysis demonstrated three clusters/subtypes corresponding to AD, SQC, and NE (FIG. 2). The predetermined LSP 3-subtype centroid predictor was then applied to all 4 datasets, and results were compared with tumor morphologic classifications. Percent agreement and Fleiss' kappa were calculated for each dataset (Table 17). The percent agreement ranged from 78%-91% and kappa's from 0.57-0.85.

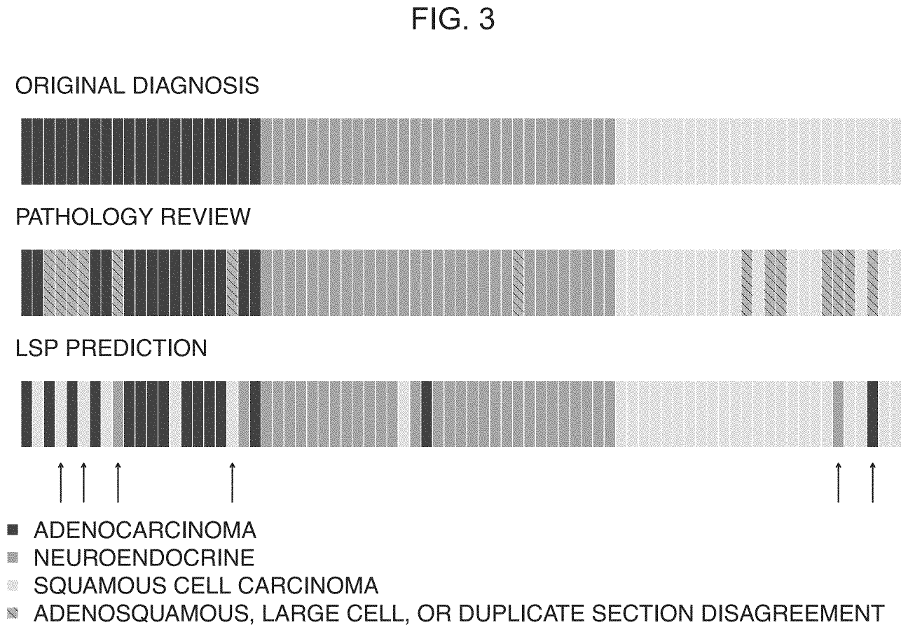

As another means of assessing independent pathology agreement, the agreement of blinded pathology review of the 77 FFPE lung tumors with the original morphologic diagnosis was found to be 82% (63/77). In 12/77 cases, blinded duplicate slides provided conflicting results and in 10/77 cases, at least one of the duplicates had a non-definitive pathological subtype classification of “Adenosquamous”, “Large Cell”, or “High grade poorly differentiated carcinoma”. Comparison of the original morphologic diagnosis, blinded pathology review, and gene expression LSP subtype call for each of the 77 samples is shown in FIG. 3. Details of discordant sample overlap (i.e., 6 samples where tumor subtype disagreed with original morphology diagnosis by both path review and gene expression LSP call) are provided in Table 18. Overall, these concordance values of LSP relative to the original pathology calls were at least as great as the concordance between any two pathologists (Grilley et al. Arch Pathol Lab Med 2013; 137: 32-40; Thunnissen et al. Virchows Arch 2012; 461(6):629-38. Doi: 10.1007/s00428-012-1234-x. Epub 2012 Oct. 12; Thunnissen et al. Mod Pathol 2012; 25(12):1574-83. Doi: 10.1038/modpathol.2012.106; each of which is incorporated by reference herein for all purposes) thus suggesting that the assay described herein performs at least as well as a trained pathologist.

In this study, LSP provided reliable subtype classifications, validating its performance across multiple gene expression platforms, and even when using FFPE specimens. Hierarchical clustering of the newly assayed FFPE samples demonstrated good separation of the 3 subtypes (AC, SQC, and NE) based on the levels of 52 classifier biomarkers. Concordance with morphology diagnosis when using the LSP centroids was greatest in the TCGA RNAseq dataset (agreement=91%), possibly due to the very extensive pathology review and accuracy of the histologic diagnosis associated with TCGA samples as compared to other datasets. Agreement was lowest (78%) in the Agilent dataset, which may have been affected by the reduced number of genes that were available for that analysis. Overall, the LSP assay displayed a higher concordance with the original morphology diagnosis than the pathology review in all datasets except in the Agilent dataset, in which only 47 genes, rather than 52, were present for the analysis.

In the FFPE samples where blinded pathology re-review was possible, results suggested that pathology calls were not always consistent with the original diagnosis, nor were they necessarily consistent in the duplicate slides provided from each sample. For a subset of samples (n=6), both the pathology re-review and the LSP gene expression analysis suggested the same alternate diagnosis, leading one to question the accuracy of the original morphologic diagnosis, which was our “gold standard”.

In this study, there were a low number of NE tumor samples in the Affymetrix dataset, and an absence of NE samples in both the Agilent and TCGA datasets. This was partially overcome by a relatively high number of NE samples in the FFPE sample set (31/77), thus providing a good test of the LSP signature's ability to identify NE samples. Another limitation of the study relates to the blinded pathology re-review. The blinded pathology review was based on two imaged sections and did not reflect usual histology standard practice where multiple sections/blocks and potentially IHC stains would have been available to make a diagnosis.

### INCORPORATION BY REFERENCE

The following references are incorporated by reference in their entireties for all purposes.
1. American Cancer Society. Cancer Facts and Figures, 2014.
2. National Comprehensive Cancer Network (NCCN) Clinical Practice Guideline in Oncology. Non-Small Cell Lung Cancer. Version 1.2015.
3. AVASTIN® (Bevacizumab) Genetech Inc, San Francisco, Calif. prescribing information. http://www.gene.com/download/pdf/avastin_prescribing.pdf
4. ALIMTA® (Pemetrexed disodium) Eli Lilly & Co., Indianapolis, Ind. prescribing information. http://pi.lilly.com/us/alimta-pi.pdf
5. Grilley Olson J E, Hayes D N, Moore D T, et al. Validation of interobserver agreement in lung cancer assessment: hematoxylin-eosin diagnostic reproducibility for non-small cell lung cancer. Arch Pathol Lab Med 2013; 137: 32-40
6. Thunnissen E, Boers E, Heideman D A, et al. Correlation of immunohistochemical staining p63 and TTF-1 with EGFR and K-ras mutational spectrum and diagnostic reproducibility in non small cell lung carcinoma. Virchows Arch 2012; 461(6):629-38. Doi: 10.1007/s00428-012-1234-x. Epub 2012 Oct. 12.
7. Thunnissen E, Beasley M B, Borczuk A C, et al. Reproducibility of histopathological subtypes and invasion in pulmonary adenocarcinoma. An international interobserver study. Mod Pathol 2012; 25(12):1574-83. Doi: 10.1038/modpathol.2012.106.
8. Rekhtman N, Ang D C, Sima C S, Travis W D, Moreira A L. Immunohistochemical algorithm for differentiation of lung adenocarcinoma and squamous cell carcinoma based on large series of whole-tissue sections with validation in small specimens. Modern Path. 2011; 24:1348-1359.
9. Travis W D, Brambilla E, Riley G J, New pathologic classification of lung cancer: relevance for clinical practice and clinical trials. J Clin Oncol 2013; 31:992-1001.
10. Thunnissen E, Noguchi M, Aisner S, et al. Reproducibility of histopathological diagnosis in poorly differentiated NSCLC: an international multiobserver study. J Thorac Oncol 2014; 9(9): 1354-62. doi:10. 1097/JTO.0000000000000264.
11. Travis W D and Rekhtman N. Pathological diagnosis and classification of lung cancer in small biopsies and cytology: strategic management of tissue for molecular testing. Sem Resp and Crit Care Med 2011; 32(1): 22-31.
12. Travis W D, Brambilla E, Noguchi M et al. Diagnosis of lung adenocarcinoma in small biopsies and cytology: implications of the 2011 International Association for the Study of Lung Cancer/American Thoracic Society/European Respiratory Society classification. Arch Pathol Lab Med 2013; 137(5):668-84.
13. Tang E R, Schreiner A. M., Bradley B P. Advances in lung adenocarcinoma classification: a summary of the new international multidisciplinary classification system (IASLC/ATS/ERS). J Thorac Dis 2014; 6(S5):S489-S501.
14. The Clinical Lung Cancer Genome Project (CLCGP) and Network Genomic Medicine (NGM). A genomics-based classification of human lung tumors. Sci Transl Med 5, 209ra153 (2013); doi: 10.1126/scitranslmed.3006802.
15. Cancer Genome Atlas Research Network. “Comprehensive genomic characterization of squamous cell lung cancers.” Nature 489.7417 (2012): 519-525.
16. Cancer Genome Atlas Research Network. Comprehensive molecular profiling of lung adenocarcinoma. Nature 511.7511 (2014): 543-550.
17. Hayes D N, Monti S, Parmigiani G, et al. Gene expression profiling reveals reproducible human lung adenocarcinoma subtypes in multiple independent patient cohorts. J Clin Oncol 2006. 24(31): 5079-5090.
18. Shedden K, Taylor J M G, Enkemann S A, et al. Gene expression-based survival prediction in lung adenocarcinoma: a multi-site, blinded validation study: director's challenge consortium for the molecular classification of lung adenocarcinoma. Nat Med 2008. 14(8): 822-827. doi: 10.1038/nm.1790.
19. Wilkerson, Matthew D., et al. Lung squamous cell carcinoma mRNA expression subtypes are reproducible, clinically important, and correspond to normal cell types. Clinical Cancer Research 16.19 (2010): 4864-4875.
20. Wilkerson M, Yin X, Walter V, et al. Differential pathogenesis of lung adenocarcinoma subtypes involving sequence mutations, copy number, chromosomal instability, and methylation. PLoS ONE. 2012; 7(5) e36530. Doi:10.1371/journal.pone.0036530.
21. Wilkerson M D, Schallheim J M, Hayes D N, et al. Prediction of lung cancer histological types by RT-qPCR gene expression in FFPE specimens. J Molec Diagn 2013; 15:485-497.
22. Roepman P, et al. An immune response enriched 72-gene prognostic profile for early-stage non-small-cell lung cancer. Clinical Cancer Research 15.1 (2009): 284-290.
23. Lee E S, et al. Prediction of recurrence-free survival in postoperative non-small cell lung cancer patients by using an integrated model of clinical information and gene expression.” Clinical Cancer Research 14.22 (2008): 7397-7404.
24. International Genomics Consortium [http://www.intgen.org]
25. Rousseaux S, et al. Ectopic activation of germline and placental genes identifies aggressive metastasis-prone lung cancers. Science translational medicine 5.186 (2013): 186ra66-186ra66.
26. Bild A H, Yao G, Chang J T, et al. Oncogenic pathway signatures in human cancers as a guide to targeted therapies. Nature 439.7074 (2006): 353-357.
27. Faruki H, Miglarese M, Mayhew G, et al. Validation of a RT-PCR Gene Expression Assay for Subtyping Lung Tumor Samples. Abstract #4222. Presented at the Association of Molecular Pathology Annual Meeting in Baltimore, Md. Nov. 12-15, 2014.
28. Li B, and Dewey C N. RSEM: accurate transcript quantification from RNA-Seq data with or without a reference genome. BMC Bioinformatics 2011, 12:323 doi:10.1186/1471-2105-12-323
29. Yang Y H, Dudoit S, Luu P, et al. Normalization for cDNA microarray data: a robust composite method addressing single and multiple slide systematic variation. Nucleic Acids Research 2002; 30(4): e15.
30. Hubbell E, Liu W, and Mei R. Robust estimators for expression analysis. Bioinformatics (2002) 18 (12): 1585-1592. doi:10.1093/bioinformatics/18.12.1585.
31. Rekhtman N, Tafe L J, Chaft J E, et al. Distinct profile of driver mutations and clinical features in immunomarker-defined subsets of pulmonary large-cell carcinoma. Mod Pathol 2013; 26(4): 511-22. doi: 10.1038/modpathol.2012.195.
32. Rossi G, Mengoli M C, Cavazza A, et al. Large cell carcinoma of the lung: clinically oriented classification integrating immunohistochemistry and molecular biology. Virchows Arch. 2014; 464(1): 61-8. doi: 10.1007/s00428-013-15012-6.
33. Travis W D, Brambilla E, Noguchi M, Nicholson A G, Geisinger K R, Yatabe Y, et al. 2011; International Association for the study of lung cancer/American Thoracic Society/European Respiratory Society International multidisciplinary classification of lung adenocarcinoma. J Thorac Oncol, 6:244-285.

TABLE 17
Subtype prediction and agreement with morphologic diagnosis for multiple validation datasets
analyzed by the gene expression LSP gene signature. (Results shown below were in part
based upon data generated by the TCGA Research Network: http://cancergenome.nih.gov/).

| | Prediction | | | |
|---|---|---|---|---|
| | TCGA RNAseq | Agilent | Affymetrix | UNC FFPE |
| Histology Diagnosis | AD ∥ NE ∥ SQ ∥ Sum | AD ∥ NE ∥ SQ ∥ Sum | AD ∥ NE ∥ SQ ∥ Sum | AD ∥ NE ∥ SQ ∥ Sum |
| Adenocarcinoma (AD) | 419 ∥ 21 ∥ 28 ∥ 468 | 131 ∥ 6 ∥ 37 ∥ 174 | 248 ∥ 0 ∥ 16 ∥ 264 | 13 ∥ 2 ∥ 6 ∥ 21 |
| Neuroendocrine (NE)* | NA ∥ NA ∥ NA ∥ NA | NA ∥ NA ∥ NA ∥ NA | 2 ∥ 43 ∥ 2 ∥ 47 | 1 ∥ 29 ∥ 1 ∥ 31 |
| Squamous cell (SQ) | 22 ∥ 11 ∥ 450 ∥ 483 | 27 ∥ 1 ∥ 120 ∥ 148 | 26 ∥ 0 ∥ 201 ∥ 227 | 1 ∥ 1 ∥ 23 ∥ 25 |
| Sum | 441 ∥ 32 ∥ 478 ∥ 951 | 158 ∥ 7 ∥ 157∥322 | 276 ∥ 43 ∥ 219 ∥ 538 | 15 ∥ 32 ∥ 30 ∥ 77 |
| % Agreement | 91% (869/951) | 78% (251/322) | 91% (492/538) | 84% (65/77) |
| Kappa | 0.83 | 0.57 | 0.85 | 0.76 |

*includes small cell carcinoma and carcinoid

TABLE 18
Original morphology diagnosis, blinded path review, and LSP subtype
result details for 6 FFPE samples, in which both path review and LSP
predicted subtype disagreed with the original morphologic diagnosis.

| Sample | Orig Morph | Path review | Path review | LSP Subtype |
|---|---|---|---|---|
| # | Diag | #1 | #2 | Prediction |
| #021 | adenocarcinoma | adenosquamous | adenosquamous | Squamous cell |
| | | | | carcinoma |
| #023 | adenocarcinoma | adenocarcinoma | Large cell | Squamous cell |
| | | | carcinoma | carcinoma |
| #026 | adenocarcinoma | adenocarcinoma | carcinoid | neuroendocrine |
| #036 | adenocarcinoma | adenosquamous | Squamous cell | Squamous cell |
| | | | carcinoma | carcinoma |
| #043 | Squamous cell | Large cell | Squamous cell | neuroendocrine |
| | carcinoma | carcinoma | carcinoma | |
| #046 | Squamous cell | adenocarcinoma | Large cell | adenocarcinoma |
| | carcinoma | | carcinoma | |

### Example 3

### Survival Differences of Adenocarcinoma Lung Tumors with Squamous Cell Carcinoma or Neuroendocrine Profiles by Gene Expression Subtyping

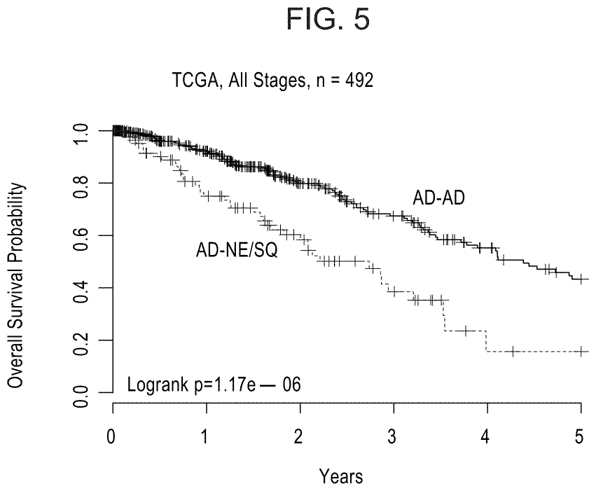

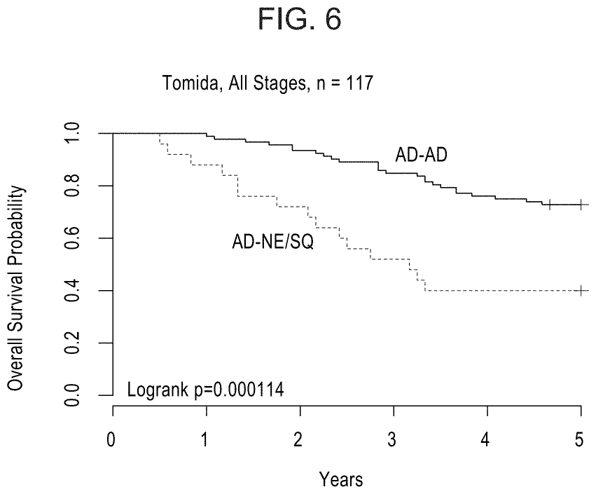

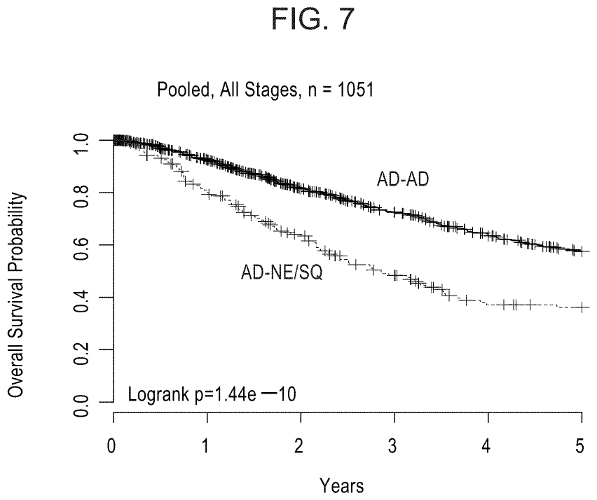

As shown in FIGS. 4-7, the Lung Subtype Panel (LSP) 3-class (Adenocarcinoma (AD), Squamous Cell Carcinoma (SQ), and Neuroendocrine (NE)) nearest centroid predictor developed in array data and described herein was applied to histology defined AD samples of all stages in the Director's Challenge (Shedden et al., Affy array, n=442, FIG. 4), TCGA (RNAseq, n=492, FIG. 5), and Tomida et al. (Agilent array, n=117, FIG. 6) datasets. Each histology defined AD sample was predicted as AD, SQ, or NE based on the LSP nearest centroid predictor. Kaplan Meier plots (FIGS. 4-7) and log rank tests for each dataset (FIGS. 4-6) and the pooled datasets (FIG. 7) were used to assess and compare 5-year overall survival in two groups, those that were histologically and gene expression (GE) concordant (AD-AD) and those that were histologically and GE discordant (AD predicted SQ or NE (AD-NE/SQ). Cox proportional Hazard Models were used to assess survival differences while controlling for T stage, N stage, and proliferation (as measured by the PAM 50 score; 12). The distribution of samples among the AD subtypes (Terminal Respiratory Unit (TRU), Proximal Proliferative (PP), and Proximal Inflammatory (PI)) was investigated.

For the analysis performed on the histology defined AD samples of all stages, the predictor confirmed AD subtype by GE in 80% of the histological AD samples, while the histological AD samples were called as GE subtypes of SQ and NE in 12% and 8% of cases, respectively. The AD-NE/SQ group (AD by histology and SQ or NE by gene expression LSP) had poorer survival than the AD-AD group (AD by both histology and LSP) in each data set (log rank p-value in RNAseq, Director's, and Tomida were 1.17e-06, 0,0009, and 0.0001, respectively). Pooling the 3 data sets and using a stratified cox model that allowed for different baseline hazards in each study, the hazard ratio comparing AD-NE/SQ to AD-AD was 1.84 (95% CI 1.48-2.30). When we fit the model adjusting for T stage, N stage, and proliferation score, the HR was 1.58 (95% CI 1.22-2.04). Adenosubtype profiling of AD-NE/SQ samples indicated that tumors were overwhelmingly of the PP or PI AD subtypes (209/213).

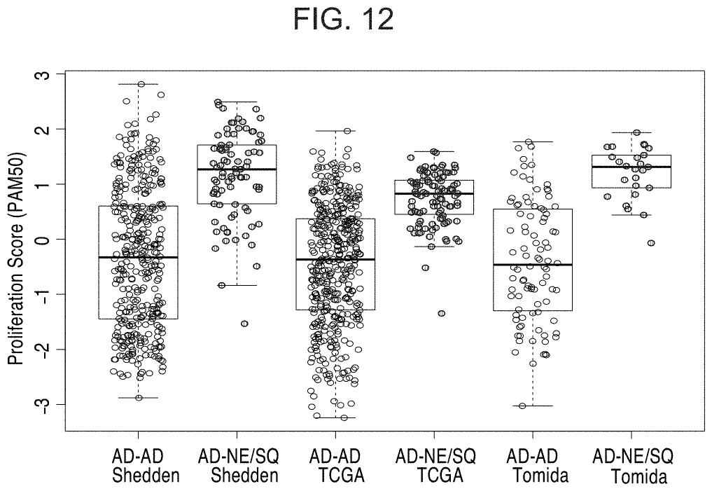

Overall, ˜20% histologic-defined lung adenocarcinoma (AD) differ in gene expression profiles. Histology-GE discordant AD tumors show worse survival than concordant cases. Survival differences may be partially explained by elevated proliferation score (see FIG. 12). Survival differences may be due to tumor biology and/or to variable response to standard AD management regimens. Further, gene expression tumor subtyping may provide valuable clinical information identifying a subset of AD samples with poor prognosis. Poor prognosis adenocarcinoma samples belong to the PI and PP adenocarcinoma subtypes, and demonstrate elevated proliferation scores. This subset of AD tumors may be less responsive to standard adenocarcinoma management.

### INCORPORATION BY REFERENCE

The following references are incorporated by reference in their entireties for all purposes.
1. Shedden K, et al. Nat Med 2008. 14(8): 822-827.
2. TCGA Cancer Nature 2014: 511(7511): 543-550
3. Tomida S, J Clin Oncol 2009; 27(17): 2793-99.
4. Neilsen T O. Clin Cancer Res 2010.

### Example 4

### Survival Differences of Adenocarcinoma Lung Tumors with Squamous Cell Carcinoma or Neuroendocrine Profiles by Gene Expression Subtyping

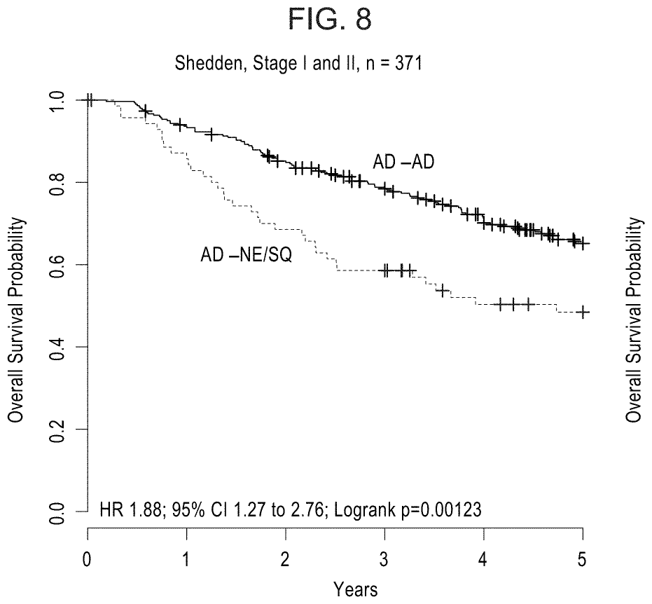

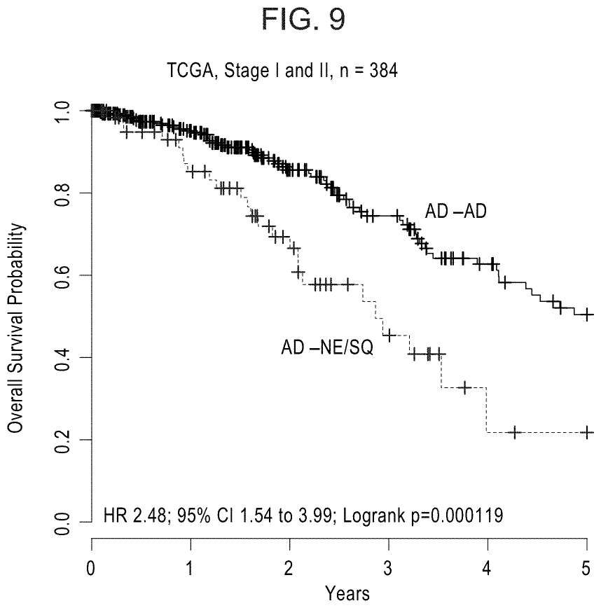

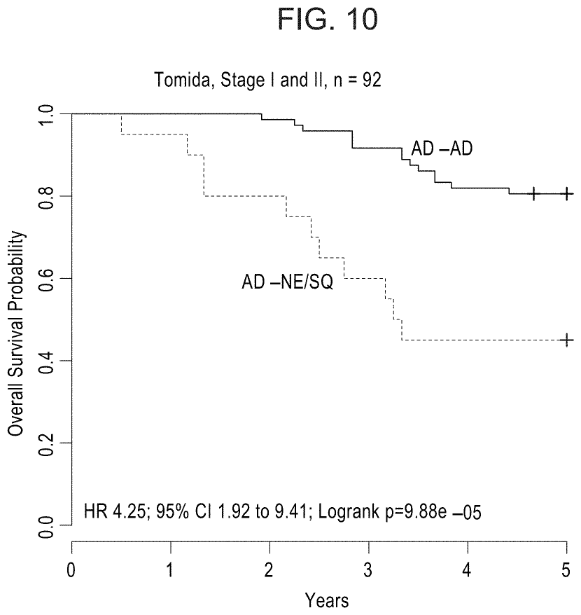

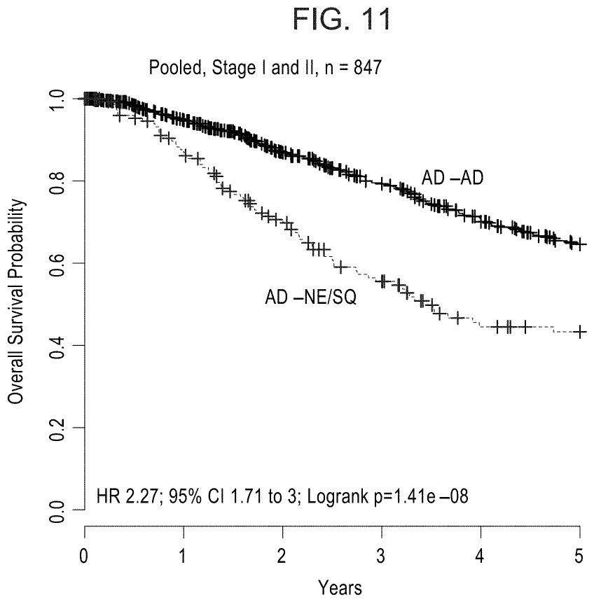

As shown in FIGS. 8-11, the Lung Subtype Panel (LSP) 3-class (Adenocarcinoma (AD), Squamous Cell Carcinoma (SQ), and Neuroendocrine (NE)) nearest centroid predictor developed in array data and described herein was applied to histology defined AD samples of stages I and II in the Director's Challenge (Shedden et al., Affy array, n=371, FIG. 8), TCGA (RNAseq, n=384, FIG. 9), and Tomida et al. (Agilent array, n=92, FIG. 10) datasets. Each histology defined AD sample was predicted as AD, SQ, or NE based on the LSP nearest centroid predictor. Kaplan Meier plots (FIGS. 8-11) and log rank tests for each dataset (FIGS. 8-10) and the pooled datasets (FIG. 11) were used to assess and compare 5-year overall survival in two groups, those that were histologically and gene expression (GE) concordant (AD-AD) and those that were histologically and GE discordant (AD predicted SQ or NE (AD-NE/SQ). Cox proportional Hazard Models were used to examine the LSP hazard ratio and to compare it with several other prognostic panels, Wilkerson et al (506 genes) Wistuba et al (31 genes), Kratz et al (11 genes) and Zhu et al (15 genes). For Wistuba et al., genes were weighted equally. For Kratz et al, genes were weighted according to the coefficients in the publication. For Zhu et al., genes were weighted −1 to +1 according to the direction of effect on OS in the TCGA AD data set. For Wilkerson et al., the risk score was calculated as distance to the TRU bronchioid) centroid. Gene mutation prevalence was examined for significantly associated mutations of lung AD and SQ. The predictor confirmed AD subtype by GE in 81% of the histological AD samples, while the histological AD samples were called as GE subtypes of SQ and NE in 12% and 7% of cases, respectively. The AD-NE/SQ group (AD by histology and SQ or NE by gene expression LSP) had poorer survival than the AD-AD group (AD by both histology and LSP) in each data set (see log rank p-value in FIGS. 8-10). Pooling the 3 data sets and using a stratified cox model that allowed for different baseline hazards in each study, the hazard ratio comparing AD-NE/SQ to AD-AD was 2.27 (95% CI 1.71 to 3) as shown in FIG. 11.

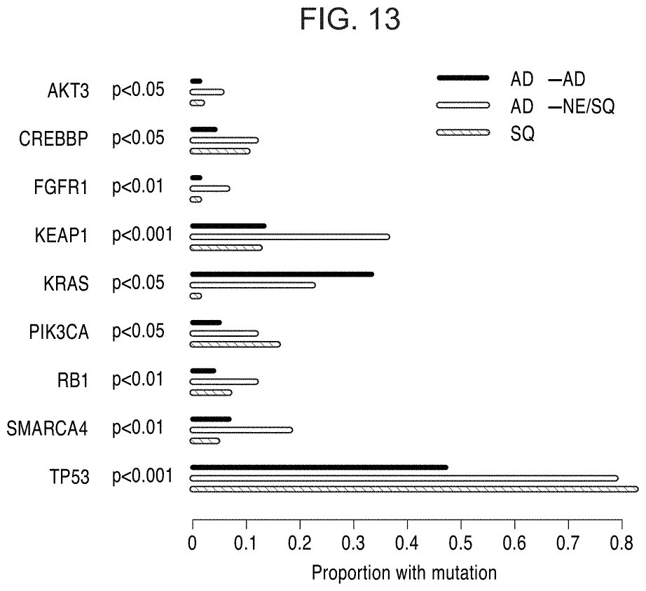

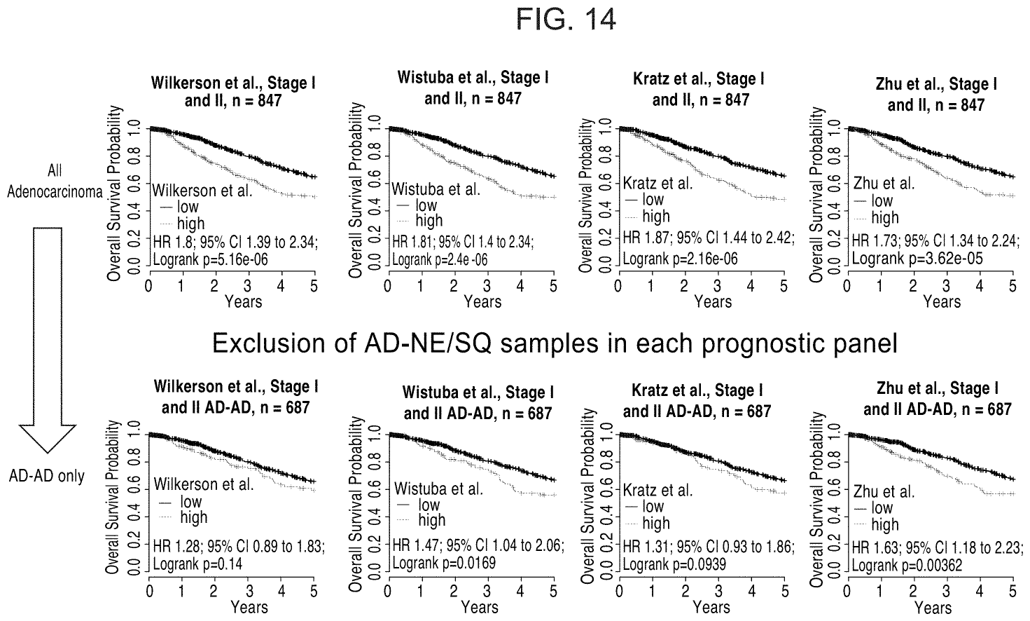

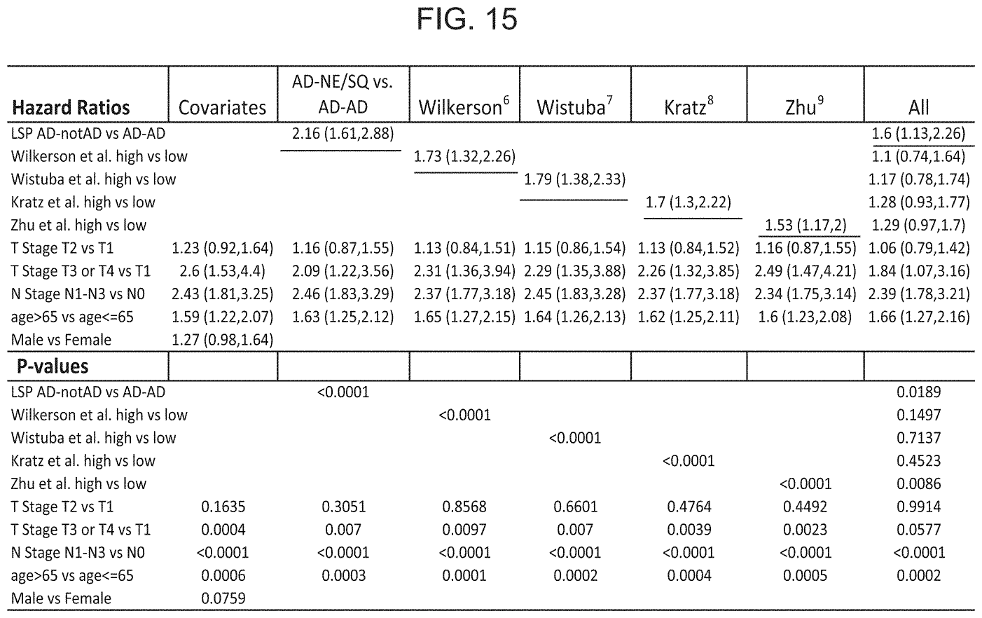

In agreement with the conclusions from Example 3, this analysis showed that ˜20% of histologically defined lung AD differ by gene expression subtype. Further, histology-GE discordant AD tumors demonstrate worse survival and are responsible for much of the prognostic risk in multiple prognostic gene signatures as shown in FIGS. 14 and 15. As shown in FIG. 13, mutation frequencies in Histology-GE discordant samples differ significantly from concordant samples for 9/48 genes evaluated. Finally, survival differences may be attributable to tumor biology and/or to variable response to standard AD management.

### INCORPORATION BY REFERENCE

The following references are incorporated by reference in their entireties for all purposes.
1. Wilkerson M D et al., J Molec Diag 2013; 15:485-497.
2. Faruki H, et al. Archives Path & Lab Med. October 2015.
3. Shedden K, et al. Nat Med 2008. 14(8): 822-827.
4. TCGA Lung AdenoC. Nature 2014: 511(7511): 543-550
5. Tomida S, J Clin Oncol 2009; 27(17): 2793-99.
6. Wilkerson M D et al. Clin Cancer Res 2013; 19(22): 6261-6271.
7. Kratz J R, et al. Lancet 2012: 379 (9818): 823-832.
8. Zhu C Q, et al. J Clin Oncol 2010; 28(29); 4417-4424.
9. TCGA Lung SQCC. Nature 2012; 489(7417): 519-525.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of assessing whether a patient's adenocarcinoma lung cancer subtype is squamoid (proximal inflammatory), bronchoid (terminal respiratory unit) or magnoid (proximal proliferative), the method comprising:
(a) probing levels of at least five classifier biomarkers of the classifier biomarkers of Table 1A, Table 1B, Table 1C, Table 2, Table 3, Table 4, Table 5 or Table 6 at in a lung cancer sample obtained from the patient at a nucleic acid level, wherein the probing step comprises;
(i) mixing the sample with five or more oligonucleotides that are substantially complementary to portions of nucleic acid molecules of the at least five classifier biomarkers of Table 1A, Table 1B, Table 1C, Table 2, Table 3, Table 4, Table 5 or Table 6 under conditions suitable for hybridization of the five or more oligonucleotides to their complements or substantial complements;
(ii) detecting whether hybridization occurs between the five or more oligonucleotides to their complements or substantial complements;
(iii) obtaining hybridization values of the at least five classifier biomarkers based on the detecting step;
(b) comparing the hybridization values of the at least five classifier biomarkers to reference hybridization value(s) from at least one sample training set, wherein the at least one sample training set comprises, (i) hybridization value(s) of the at least five biomarkers from a sample that overexpresses the at least five biomarkers, or overexpresses a subset of the at least five biomarkers, (ii) hybridization values from a reference squamoid (proximal inflammatory), bronchoid (terminal respiratory unit) or magnoid (proximal proliferative) sample, or (iii) hybridization values from an adenocarcinoma free lung sample, and
(c) classifying the adenocarcinoma sample as a squamoid (proximal inflammatory), bronchoid (terminal respiratory unit) or a magnoid (proximal proliferative) subtype based on the results of the comparing step.

2. The method of claim 1, wherein the comparing step comprises determining a correlation between the hybridization values of the at least five classifier biomarkers and the reference hybridization values.

3. The method of claim 1, wherein the comparing step further comprises determining an average expression ratio of the at least five biomarkers and comparing the average expression ratio to an average expression ratio of the at least five biomarkers, obtained from the references values in the sample training set.

4. The method of any one of claims 1-3, wherein the probing step comprises isolating the nucleic acid or portion thereof prior to the mixing step.

5. The method of any one of claims 1-4, wherein the hybridization comprises hybridization of a cDNA probe to a cDNA biomarker, thereby forming a non-natural complex.

6. The method of any one of claims 1-4, wherein the hybridization comprises hybridization of a cDNA probe to an mRNA biomarker, thereby forming a non-natural complex.

7. The method of any one of claims 1-5, wherein the probing step comprises amplifying the nucleic acid in the sample.

8. The method of any one of claims 1-7, wherein the at least five of the classifier biomarkers comprise at least 10 biomarkers, at least 20 biomarkers or at least 30 biomarkers of Table 1A, Table 1B or Table 1C.

9. The method of any one of claims 1-7, wherein the at least five of the classifier biomarkers comprise at least 10 biomarkers, at least 20 biomarkers or at least 30 biomarkers of Table 2.

10. The method of any one of claims 1-7, wherein the at least five of the classifier biomarkers comprise at least 10 biomarkers, at least 20 biomarkers or at least 30 biomarkers of Table 3.

11. The method of any one of claims 1-7, wherein the at least five of the classifier biomarkers comprise the 6 biomarkers of Table 4.

12. The method of any one of claims 1-7, wherein the at least five of the classifier biomarkers comprise the 6 biomarkers of Table 5.

13. The method of any one of claims 1-7, wherein the at least five of the classifier biomarkers comprise at least 10 biomarkers, at least 20 biomarkers or at least 30 biomarkers of Table 6.

14. The method of any one of claims 1-7, wherein the at least five of the classifier biomarkers comprise from about 10 to about 30 classifier biomarkers, or from about 15 to about 40 classifier biomarkers of Table 1A, Table 1B or Table 1C.

15. The method of any one of claims 1-7, wherein the at least five of the classifier biomarkers comprise from about 10 to about 30 classifier biomarkers, or from about 15 to about 40 classifier biomarkers of Table 2.

16. The method of any one of claims 1-7, wherein the at least five of the classifier biomarkers comprise from about 10 to about 30 classifier biomarkers, or from about 15 to about 40 classifier biomarkers of Table 3.

17. The method of any one of claims 1-7, wherein the at least five classifier biomarkers comprise from about 5 to about 30 classifier biomarkers, or from about 10 to about 30 classifier biomarkers of Table 6.

18. The method of any one of claims 1-7, wherein the at least five of the classifier biomarkers comprise each of the classifier biomarkers set forth in Table 1A, Table 1B or Table 1C.

19. The method of any one of claims 1-7, wherein the at least five of the classifier biomarkers comprise each of the classifier biomarkers set forth in Table 2.

20. The method of any one of claims 1-7, wherein the at least five of the classifier biomarkers comprise each of the classifier biomarkers set forth in Table 3.

21. The method of any one of claims 1-7, wherein the at least five of the classifier biomarkers comprise each of the classifier biomarkers set forth in Table 6.

22. The method of any one of claims 1-21, wherein the sample comprises lung cells embedded in paraffin.

23. The method of any one of claims 1-21, wherein the sample is a fresh frozen sample.

24. The method according to any one of claims 1-21, wherein the lung tissue sample is selected from a formalin-fixed, paraffin-embedded (FFPE) lung tissue sample, fresh and a frozen tissue sample.

25. The method of claim 18, wherein the at least five of the classifier biomarkers comprise each of the classifier biomarkers set forth in Table 1A.

26. The method of claim 18, wherein the at least five of the classifier biomarkers comprise each of the classifier biomarkers set forth in Table 1B.

27. The method of claim 18, wherein the at least five of the classifier biomarkers comprise each of the classifier biomarkers set forth in Table 1C.

28. A method for determining a disease outcome for a patient suffering from lung cancer, the method comprising: determining a subtype of the lung cancer through gene expression analysis of a first sample obtained from the patient to produce a gene expression based subtype; determining the subtype of the lung cancer through a morphological analysis of a second sample obtained from the patient to produce a morphological based subtype; and comparing the gene expression based subtype to the morphological based subtype, wherein a presence or absence of concordance between the gene expression based subtype and the morphological based subtype is predictive of the disease outcome.

29. The method of claim 28, wherein discordance between the gene expression based subtype and morphological based subtype is predictive of a poor disease outcome.

30. The method of claim 28 or 29, wherein the disease outcome is overall survival.

31. The method of any of claims 28-30, wherein the gene expression base subtype and/or morphological based subtype is adenocarcinoma, squamous cell carcinoma, or neuroendocrine.

32. The method claim 31, wherein the neuroendocrine encompasses small cell carcinoma and carcinoid.

33. The method of any one of claims 28-32, wherein the first sample and/or the second sample is a formalin-fixed, paraffin-embedded (FFPE) lung tissue sample, fresh, or a frozen tissue sample.

34. The method of any one of claims 28-33, wherein the first sample and the second sample are portions of an identical sample.

35. The method of any one of claims 28-34, wherein the gene expression analysis comprises determining expression levels of at least five classifier biomarkers in Table 1A, Table 1B, Table 1C, Table 2, Table 3, Table 4, Table 5 or Table 6 at a nucleic acid level in the first sample by performing RNA sequencing, reverse transcriptase polymerase chain reaction (RT-PCR) or hybridization based analyses.

36. The method of claim 35, wherein the RT-PCR is quantitative real time reverse transcriptase polymerase chain reaction (qRT-PCR).

37. The method of claim 35, wherein the RT-PCR is performed with primers specific to the at least five classifier biomarkers; comparing the detected levels of expression of the at least five classifier biomarkers of Table 1A, Table 1B, Table 1C, Table 2, Table 3, Table 4, Table 5 or Table 6 to the expression of the at least five classifier biomarkers in at least one sample training set(s), wherein the at least one sample training set comprises expression data of the at least five classifier biomarkers of Table 1A, Table 1B, Table 1C, Table 2, Table 3, Table 4, Table 5 or Table 6 from a reference adenocarcinoma sample, expression data of the at least five classifier biomarkers of Table 1A, Table 1B, Table 1C, Table 2, Table 3, Table 4, Table 5 or Table 6 from a reference squamous cell carcinoma sample, expression data of the at least five classifier biomarkers of Table 1A, Table 1B, Table 1C, Table 2, Table 3, Table 4, Table 5 or Table 6 from a reference neuroendocrine sample, or a combination thereof; and classifying the first sample as an adenocarcinoma, squamous cell carcinoma, or a neuroendocrine subtype based on the results of the comparing step.

38. The method of claim 37, wherein the comparing step comprises applying a statistical algorithm which comprises determining a correlation between the expression data obtained from the first sample and the expression data from the at least one training set(s); and classifying the first sample as an adenocarcinoma, squamous cell carcinoma, or a neuroendocrine subtype based on the results of the statistical algorithm.

39. The method of claim 37 or 38 , wherein the primers specific for the at least five classifier biomarkers are forward and reverse primers listed in Table 1A, Table 1B, Table 1C, Table 2, Table 3, Table 4, Table 5 or Table 6.

40. The method of claim 35, wherein the hybridization based analysis comprises:
(a) probing the levels of at least five classifier biomarkers of Table 1A, Table IB, Table 1C, Table 2, Table 3, Table 4, Table 5 or Table 6 in a lung cancer sample obtained from the patient at the nucleic acid level, wherein the probing step comprises;
(i) mixing the sample with five or more oligonucleotides that are substantially complementary to portions of nucleic acid molecules of the at least five classifier biomarkers of Table 1A, Table IB, Table 1C, Table 2, Table 3, Table 4, Table 5 or Table 6 under conditions suitable for hybridization of the five or more oligonucleotides to their complements or substantial complements;
(ii) detecting whether hybridization occurs between the five or more oligonucleotides to their complements or substantial complements;
(iii) obtaining hybridization values of the at least five classifier biomarkers based on the detecting step;
(b) comparing the hybridization values of the at least five classifier biomarkers to reference hybridization value(s) from at least one sample training set, wherein the at least one sample training set comprises hybridization values from a reference adenocarcinoma sample, hybridization values from a reference squamous cell carcinoma sample, hybridization values from a reference neuroendocrine sample, or a combination thereof; and
(c) classifying the lung cancer sample as a adenocarcinoma, squamous cell carcinoma, or a neuroendocrine subtype based on the results of the comparing step.

41. The method of claim 40, wherein the comparing step comprises determining a correlation between the hybridization values of the at least five classifier biomarkers and the reference hybridization values.

42. The method of claim 40, wherein the comparing step further comprises determining an average expression ratio of the at least five biomarkers and comparing the average expression ratio to an average expression ratio of the at least five biomarkers, obtained from the references values in the sample training set.

43. The method of any one of claims 40-42, wherein the probing step comprises isolating the nucleic acid or portion thereof prior to the mixing step.

44. The method of any one of claims 40 -43, wherein the hybridization comprises hybridization of a cDNA probe to a cDNA biomarker, thereby forming a non-natural complex.

45. The method of any one of claims 40 -43, wherein the hybridization comprises hybridization of a cDNA probe to an mRNA biomarker, thereby forming a non-natural complex.

46. The method of claim 35, wherein the at least five of the classifier biomarkers comprise at least 10 biomarkers, at least 20 biomarkers or at least 30 biomarkers of Table 1A, Table 1B or Table 1C.

47. The method of claim 35, wherein the at least five of the classifier biomarkers comprise at least 10 biomarkers, at least 20 biomarkers or at least 30 biomarkers of Table 2.

48. The method of claim 35, wherein the at least five of the classifier biomarkers comprise at least 10 biomarkers, at least 20 biomarkers or at least 30 biomarkers of Table 3.

49. The method of claim 35, wherein the at least five of the classifier biomarkers comprise the 6 biomarkers of Table 4.

50. The method of claim 35, wherein the at least five of the classifier biomarkers comprise the 6 biomarkers of Table 5.

51. The method of claim 35, wherein the at least five of the classifier biomarkers comprise at least 10 biomarkers, at least 20 biomarkers or at least 30 biomarkers of Table 6.

52. The method of claim 35, wherein the at least five of the classifier biomarkers comprise from about 10 to about 30 classifier biomarkers, or from about 15 to about 40 classifier biomarkers of Table 1A, Table 1B or Table 1C.

53. The method of claim 35, wherein the at least five of the classifier biomarkers comprise from about 10 to about 30 classifier biomarkers, or from about 15 to about 40 classifier biomarkers of Table 2.

54. The method of claim 35, wherein the at least five of the classifier biomarkers comprise from about 10 to about 30 classifier biomarkers, or from about 15 to about 40 classifier biomarkers of Table 3.

55. The method of claim 35, wherein the at least five classifier biomarkers comprise from about 5 to about 30 classifier biomarkers, or from about 10 to about 30 classifier biomarkers of Table 6.

56. The method of claim 35, wherein the at least five of the classifier biomarkers comprise each of the classifier biomarkers set forth in Table 1A, Table 1B or Table 1C.

57. The method of claim 35, wherein the at least five of the classifier biomarkers comprise each of the classifier biomarkers set forth in Table 2.

58. The method of claim 35, wherein the at least five of the classifier biomarkers comprise each of the classifier biomarkers set forth in Table 3.

59. The method of claim 35, wherein the at least five of the classifier biomarkers comprise each of the classifier biomarkers set forth in Table 6.

60. The method of claim 56, wherein the at least five of the classifier biomarkers comprise each of the classifier biomarkers set forth in Table 1A.

61. The method of claim 56, wherein the at least five of the classifier biomarkers comprise each of the classifier biomarkers set forth in Table 1B.

62. The method of claim 56, wherein the at least five of the classifier biomarkers comprise each of the classifier biomarkers set forth in Table 1C.

63. The method of any one of claims 28-62, wherein the morphological analysis of the second sample is a histological analysis.

64. A method of assessing whether a lung tissue sample from a human patient is a squamoid (proximal inflammatory), bronchoid (terminal respiratory unit) or magnoid (proximal proliferative) adenocarcinoma lung cancer subtype, the method comprising:
detecting expression levels of at least five of the classifier biomarkers of Table 1A, Table 1B, Table 1C, Table 2, Table 3, Table 4, Table 5 or Table 6 at the nucleic acid level by RNA-seq, a reverse transcriptase polymerase chain reaction (RT-PCR) or a hybridization assay with oligonucleotides specific to the classifier biomarkers;
comparing the detected levels of expression of the at least five of the classifier biomarkers of Table 1A, Table 1B, Table 1C, Table 2, Table 3, Table 4, Table 5 or Table 6 to the expression levels of the at least five of the classifier biomarkers from at least one sample training set, wherein the at least one sample training set comprises, (i) expression levels(s) of the at least five biomarkers from a sample that overexpresses the at least five biomarkers, or overexpresses a subset of the at least five biomarkers, (ii) expression levels from a reference squamoid (proximal inflammatory), bronchoid (terminal respiratory unit) or magnoid (proximal proliferative) sample, or (iii) expression levels from an adenocarcinoma free lung sample; and
classifying the lung tissue sample as a squamoid (proximal inflammatory), bronchoid (terminal respiratory unit) or a magnoid (proximal proliferative) subtype based on the results of the comparing step.

65. The method of claim 64, wherein the comparing step comprises applying a statistical algorithm which comprises determining a correlation between the expression data obtained from the lung tissue sample and the expression data from the at least one training set(s); and classifying the lung tissue sample as a squamoid (proximal inflammatory), bronchoid (terminal respiratory unit) or a magnoid (proximal proliferative) subtype based on the results of the statistical algorithm.

66. The method of claim 64 or 65, wherein the lung tissue sample is selected from a formalin-fixed, paraffin-embedded (FFPE) lung tissue sample, fresh and a frozen tissue sample.

67. The method of claim 64, wherein the comparing step further comprises determining an average expression ratio of the at least five biomarkers and comparing the average expression ratio to an average expression ratio of the at least five biomarkers, obtained from the references values in the sample training set.

68. The method of any one of claims 64-67, wherein the at least five of the classifier biomarkers comprise at least 10 biomarkers, at least 20 biomarkers or at least 30 biomarkers of Table 1A, Table 1B or Table 1C.

69. The method of any one of claims 64-67, wherein the at least five of the classifier biomarkers comprise at least 10 biomarkers, at least 20 biomarkers or at least 30 biomarkers of Table 2.

70. The method of any one of claims 64-67, wherein the at least five of the classifier biomarkers comprise at least 10 biomarkers, at least 20 biomarkers or at least 30 biomarkers of Table 3.

71. The method of any one of claims 64-67, wherein the at least five of the classifier biomarkers comprise the 6 biomarkers of Table 4.

72. The method of any one of claims 64-67, wherein the at least five of the classifier biomarkers comprise the 6 biomarkers of Table 5.

73. The method of any one of claims 64-67, wherein the at least five of the classifier biomarkers comprise at least 10 biomarkers, at least 20 biomarkers or at least 30 biomarkers of Table 6.

74. The method of any one of claims 64-67, wherein the at least five of the classifier biomarkers comprise from about 10 to about 30 classifier biomarkers, or from about 15 to about 40 classifier biomarkers of Table 1A, Table 1B or Table 1C.

75. The method of any one of claims 64-67, wherein the at least five of the classifier biomarkers comprise from about 10 to about 30 classifier biomarkers, or from about 15 to about 40 classifier biomarkers of Table 2.

76. The method of any one of claims 64-67, wherein the at least five of the classifier biomarkers comprise from about 10 to about 30 classifier biomarkers, or from about 15 to about 40 classifier biomarkers of Table 3.

77. The method of any one of claims 64-67, wherein the at least five classifier biomarkers comprise from about 5 to about 30 classifier biomarkers, or from about 10 to about 30 classifier biomarkers of Table 6.

78. The method of any one of claims 64-67, wherein the at least five of the classifier biomarkers comprise each of the classifier biomarkers set forth in Table 1A, Table 1B or Table 1C.

79. The method of any one of claims 64-67, wherein the at least five of the classifier biomarkers comprise each of the classifier biomarkers set forth in Table 2.

80. The method of any one of claims 64-67, wherein the at least five of the classifier biomarkers comprise each of the classifier biomarkers set forth in Table 3.

81. The method of any one of claims 64-67, wherein the at least five of the classifier biomarkers comprise each of the classifier biomarkers set forth in Table 6.

82. The method of claim 78, wherein the at least five of the classifier biomarkers comprise each of the classifier biomarkers set forth in Table 1A.

83. The method of claim 78, wherein the at least five of the classifier biomarkers comprise each of the classifier biomarkers set forth in Table 1B.

84. The method of claim 78, wherein the at least five of the classifier biomarkers comprise each of the classifier biomarkers set forth in Table 1C.
